(19) United States
(12) Patent Application Publication
(10) Pub. No.: US 2024/0189440 A1
(43) Pub. Date: Jun. 13, 2024

(54) TLR7 AND TLR8 AGONISTS FOR THE TREATMENT OF CANCER AND/OR INFECTIOUS DISEASES

(71) Applicant: THE RESEARCH FOUNDATION FOR THE STATE UNIVERSITY OF NEW YORK, Albany, NY (US)

(72) Inventors: Lawrence Nathan TUMEY, Vestal, NY (US); Emmanuel OLAWODE, El Mirage, AZ (US)

(73) Assignee: THE RESEARCH FOUNDATION FOR THE STATE UNIVERSITY OF NEW YORK, Albany, NY (US)

(21) Appl. No.: 18/547,594

(22) PCT Filed: Mar. 1, 2022
(86) PCT No.: PCT/US2022/070889

§ 371 Date: Aug. 23, 2023

Related U.S. Application Data
(60) Provisional application No. 63/155,489, filed on Mar. 2, 2021.

(51) Int. Cl. A61K 47/68; A61P 35/00

(52) U.S. Cl. CPC A61K 47/6803; A61K 47/6855; A61P 35/00

(57) ABSTRACT

Disclosed herein are compounds of the Formula (I) or (II) or (III):

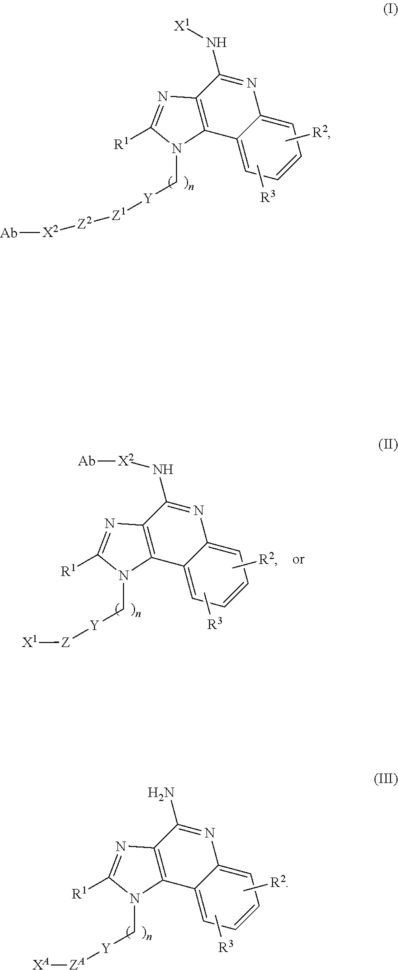

Also disclosed are methods for stimulating an immune response, inducing an anti-tumor immune response, and treating an infectious disease in a subject by administering a therapeutically effective amount of a compound disclosed herein. Also disclosed are methods of treating a tumor or abnormal cell proliferation by administering a therapeutically effective amount of a compound disclosed herein under conditions effective to treat a tumor or abnormal cell proliferation.

### CROSS-REFERENCE TO RELATED APPLICATION

This application claims priority of U.S. provisional application 63/155,489, filed Mar. 2, 2021, the entire disclosure of which is hereby incorporated herein by reference.

### GOVERNMENT RIGHTS STATEMENT

This invention was made with partial U.S. Government support under GRANT13239646 and GRANT12801094 awarded by National Institutes of Health. The U.S. Government has certain rights in the invention.

### FIELD OF THE INVENTION

The invention relates to agonists of toll-like receptors (TLRs) and the targeted delivery of agonists of toll-like receptors (TLRs). In particular, compounds of the present invention are TLR 7 and/or TLR8 agonists and antibody-drug-conjugates (ADC) that allow for delivery and release of the TLR 7 and/or TLR8 agonists into desired tissues, resulting in immunoactivation. Compounds of the present invention are thus useful as therapeutic agents for treating various cancers and infectious diseases.

### BACKGROUND OF THE INVENTION

Toll-like receptors (TLRs) govern the innate immune system response through recognition of pathogen-associated molecular patterns (PAMPs). TLR7 and TLR8 are among the known human TLR endosomal receptors and are able to induce an innate immune system response and can be activated using agonists.

TLR7 and TLR8 are homologous receptors that bind and are activated by single-stranded RNA from endocytosed bacteria and viruses. Activation initiates a downstream inflammatory response, followed by creation of a complex that initiates a signaling cascade and eventually activates transcription factors such as nuclear factor kappa-light-chain-enhancer of activated B cells (NF-κB) and interferon regulatory factor 7 (IRF7). These then stimulate inflammatory cytokine and type I interferon production, which are important to many inflammatory processes. Due to differences in TLR7 and TLR8 cytokine induction profiles as well as receptor expression variability between immune cell types, activation of TLR7 or TLR8 results in unique immune responses. Likewise, secretion of cytokines from TLR7/8 activation contributes to the activation of antigen-specific T and B cells, which helps initiate the adaptive immune response. TLRs are associated with numerous immune and inflammatory conditions, and, accordingly, the ability to modulate TLR activity is a potential pathway for treatment of those conditions.

TLR agonists are immunostimulants that are often used as vaccine adjuvants (see, for instance, McGowan, D., Current Topics in Medicinal Chemistry 19:2228-2238 (2019)). These agonists activate the adaptive immune system, thus, leading to a more robust anti-viral effect. TLR agonists are also being explored as a way to “unmask” the immunosuppressive tumor environment in hopes that the immune system will recognize cancer tissue as “foreign” and thus initiate a robust anti-tumor response by the immune system. TLR agonist development is fraught with inflammation-associated side effects, as is the case with commercially available TLR7 and TLR 8 agonists (see Kieffer et al., Expert Opinion on Therapeutic Patents 30(11):825-845 (2020)).

One commercially available TLR7 agonist is imiquimod. Imiquimod has been approved for topical administration to treat genital warts (anti-viral effects), actinic keratosis, and non-melanoma skin cancers such as basal cell carcinoma (anti-tumor effects). Imiquimod application, however, is limited to topical administration due to safety concerns with system dosing. TLR agonists, such as imiquimod, if delivered systemically, result in whole-body immunostimulation, leading to acute toxicity from a cytokine-storm type of event.

Two imiquidazoline derivatives of imiquimod—resiquimod (a mixed TLR7/8 agonist also known as R848) and motolimod (a TLR8 agonist also referred to as VTX-2337)—have shown promising immunostimulatory activity in a variety of preclinical models, including models of immunotherapy for cancer (see, for instance, Prins et al., J. Immunol. 176:157-64 (2006) and Bialojan et al., Eur. J. Immunol. 49:2083-2094 (2019)). However, these agonists have not yet been approved by regulatory agencies for use in treating cancer patients. Similarly, some third-generation TLR7/TLR8 agonists have entered clinical development for the treatment of viral infection or cancer, including PF-4878691, BDC-1001, LHC165, NKTR-262, TQ-A3334, RO7119929, DSP-0509, BNT411, and NJH395 (see, for instance, Hanten et al., BMC Immunol. 9:39 (2008); Weigel et al., Am. J. Hematol. 87:953-956 (2012); Dudek et al., Clin. Cancer Res. 13:7119-7125 (2007); Fidock et al., Clin. Pharmacol. Ther. 89:821-829 (2011); Inglefield et al., J. Interferon Cytokine Res. 28:253-263 (2008); Astry et al., J. Clin. Pharmacol. 48:755-762 (2008); Dummer et al., Clin. Cancer Res. 14:856-864 (2008); Harrison et al., J. Clin. Pharmacol. 47:962-969 (2007); Bryden et al., Sci. Transl. Med. 12:eaax2421 (2020); Cromarty et al., Front Immunol. 10:1705 (2019); and LaRue et al., Nat. Rev. Urol. 10:537-545 (2013)). However, none of these molecules have obtained regulatory approval for use in humans.

Clinical studies on resiquimod in the treatment of hepatis C virus were not successful (Pockros et al., J. Hepatol. 47(2):174-182 (2007)). Similarly, TLR7 agonist, GSK-2245035, was found to lack efficacy in patients with mild allergic asthma to effect a change in allergen-induced asthmatic response (Tsitoura et al., Clin. Pharmacol. Ther. 98(4):369-380 (2015)). Clinical studies on TLR7 agonist PF-4878691 were found to have a low therapeutic index in the treatment of hepatitis C virus (Fidock et al., Clin. Pharmacol. Ther. 89(6):821-829 (2011)) and other studies found that TLR7 agonist GS-9620 showed no antiviral activity in HBV infected primary human hepatocytes (Tsai et al., J. Virol. 91(8):e02166-e16 (2017) and Bam et al., Antimicrob. Agents Chemother. 61(1):e01369-e16 (2016)).

It is an ongoing problem to find methods for safe delivery of TLR7 and TLR8 agonists while maintaining therapeutic efficacy. Accordingly, there are numerous ongoing studies dedicated to the development of an improved delivery platform for TLR7 and TLR8 agonists that enable a robust local delivery without a systemic exposure. The development of resiquimod, motolimod, and other TLR7 and TLR8 agonists as immunostimulatory agents for use in cancer patients has faced difficulties and appears to stand at an impasse, at least in part due to disappointing results obtained in recent clinical testing (Frega et al., Oncoimmunology 9:1-10 (2020)). A major challenge in this field is development of efficacious molecules with adequate safety margins, as TLR agonists activate the innate immune system to elevate the body's inflammatory response (Kieffer et al., Expert Opinion on Therapeutic Patents 30(11):825-845 (2020); Patel et al., Future Virol. 9(9):811-829 (2014); and Tisoncik et al., Microbiol. Mol. Biol. Rev. 76(1):16-32 (2012)). Accordingly, the use of TLR agonists in immuno-oncology is an area of great interest, but there remains a significant need for improved TLR7 and TLR8 agonists.

The present disclosure is directed to overcoming these and other deficiencies in the art.

### SUMMARY OF THE INVENTION

Briefly, the present invention satisfies the need for targeted TLR agonists that can be delivered in a localized manner, reducing toxicity and enhancing efficacy.

The present invention provides, in a first aspect, a compound of the Formula (I) or (II)

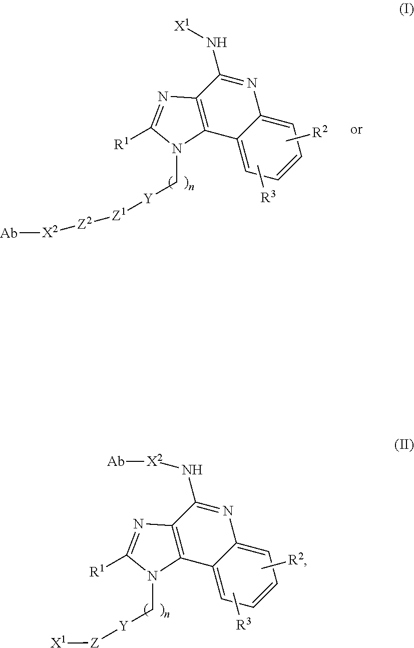

wherein:
R¹ is selected from C₁-C₁₀ alkyl, C₁-C₁₀ oxaalkyl, and C₁-C₁₀ azaalkyl;
R² and R³ are each independently selected from hydrogen, C₁-C₅ alkyl, and C₁-C₅ alkoxy;
n is 1 or 2;
Y is selected from optionally substituted aryl and optionally substituted heteroaryl;
Z¹ is selected from —NR^{Z}—, —O—, —NR^{Z}C(O)—, —NR^{Z}C(O)—O—, and —NR^{Z}SO₂—;
Z² is absent, or is selected from (C₁-C₈)hydrocarbon-NH— and a 5- to 8-membered nitrogen-containing heterocycle, wherein a nitrogen of the heterocycle is attached to X²
Z is selected from —NR^{Z}—, —NR^{Z}C(O)—, and —O—;
R^{Z} is selected from hydrogen, C₁-C₈ hydrocarbon, C₁-C₈ oxaalkyl, C₁-C₈ azaalkyl, heteroaryl, and a 5- to 8-membered heterocyclic ring;
X¹ is selected from —R^{Z}, —C(O)—R^{Z}, —C(O)—O—R^{Z}, —C(O)—N—(R^{Z})₂, —(CH₂)ₖNR^{Z}C(O)—(C₁-C₆)alkyl, —(CH₂)ₖNR^{Z}C(O)—O—(C₁-C₄)alkyl, and —SO₂—R^{Z};
k is an integer from 1 to 8;
X² comprises a cleavable or noncleavable linker; and
Ab comprises an antibody or an antibody fragment.

The present invention provides, in a second aspect, a compound of the Formula (III)

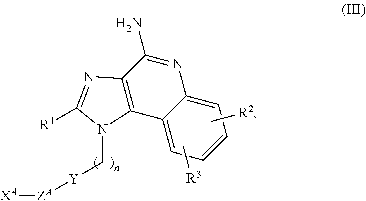

wherein:
R¹ is selected from C₁-C₁₀ alkyl, C₁-C₁₀ oxaalkyl, and C₁-C₁₀ azaalkyl;
R² and R³ are each independently selected from hydrogen, C₁-C₅ alkyl, and C₁-C₅ alkoxy;
n is 1 or 2;
Y is selected from optionally substituted aryl and optionally substituted heteroaryl;
Z^{A} is selected from —NR^{Z}—, —NR^{Z}C(O)—, —NR^{Z}C(O)—O—, —NR^{Z}C(O)—(CH₂)ₖ—NH—, —NR^{Z}C(O)—(CH₂)ₖ—O—, —C(O)—O—(CH₂)ₖ—O—, —NR^{Z}C(O)—(CH₂)ₖ—N(CH₃)—, —NR^{Z}C(O)—O—(CH₂)ₖ—NH—, —NR^{Z}C(O)—(CH₂)ₖ—NH—C(O)—O—, and —NR^{Z}SO₂—;
k is an integer from 1 to 8;
R^{Z} is selected from hydrogen, C₁-C₈ hydrocarbon, C₁-C₈ oxaalkyl, C₁-C₈ azaalkyl, heteroaryl, and a 5- to 8-membered heterocyclic ring; and
X^{A} is selected from hydrogen, C₁-C₁₀ alkyl, and —C(O)CH₃.

The present invention provides, in a third aspect, a pharmaceutical composition comprising a compound described herein and a pharmaceutically acceptable carrier, diluent, or excipient.

The present invention provides, in a fourth aspect, a method for stimulating an immune response in a subject. The method includes administering a therapeutically effective amount of a compound described herein under conditions effective to stimulate an immune response.

The present invention provides, in a fifth aspect, a method for inducing an anti-tumor immune response in a subject. The method includes administering a therapeutically effective amount of a compound described herein under conditions effective to induce an anti-tumor immune response.

The present invention provides, in a sixth aspect, a method for treating a tumor or abnormal cell proliferation in a subject. The method includes administering a therapeutically effective amount of a compound described herein under conditions effective to treat a tumor or abnormal cell proliferation.

The present invention provides, in a seventh aspect, a method for treating an infectious disease in a subject. The method includes administering a therapeutically effective amount of a compound described herein under conditions effective to treat an infectious disease.

These, and other objects, features and advantages of this invention will become apparent from the following detailed description of the various aspects of the invention taken in conjunction with the accompanying drawings.

### BRIEF DESCRIPTION OF THE DRAWINGS

FIG. 1A and FIG. 1B show the activation of NFκB in Ramos blue cells after 24 h (FIG. 1A) or 72 h (FIG. 1B) of treatment with compounds disclosed herein.

FIG. 2 demonstrates activation of the NFκB pathway in a mTLR7-HEK293 reporter cell line.

FIG. 3 shows that Anti-Her2 targeted ADCs activate a TLR7 reporter line in a media transfer study.

FIG. 4 shows that Anti-Her2 ADCs activate mTL7 in the media transfer assay. The activity is suppressed by the addition of naked antibody.

FIG. 5 demonstrates that ADCs disclosed herein show activity below 1 μg/mL in the media transfer assay.

FIG. 6 shows the evaluation of alternative linkers attached to E104 and resiquimod.

FIG. 7A and FIG. 7B demonstrate TNFα release (as measured by ELISA) induced by compounds disclosed herein from both macrophages (FIG. 7A) and monocytes (FIG. 7B).

FIG. 8 shows the results of stability studies of three ADCs disclosed herein demonstrating limited release of payload during incubation in human serum and mouse serum.

FIG. 9 shows the stability of ADCs disclosed herein in human and mouse serum.

FIG. 10 demonstrates the results of a xenograft study in mice, showing the effect on tumor size of ADCs disclosed herein.

FIG. 11 shows the effect of ADCs disclosed herein on body weight in a mouse xenograft study.

FIG. 12 shows the stability of ADCs disclosed herein in human and mouse serum.

FIG. 13 shows the stability of ADCs disclosed herein in human and mouse serum.

FIG. 14 shows the stability of ADCs disclosed herein in human and mouse serum.

FIG. 15 shows the stability of ADCs disclosed herein in human and mouse serum.

FIG. 16 shows evidence that anti-Trop2 and anti-GCC ADCs of the present invention are capable of simulating macrophages in the vicinity of antigen-expressing non-small cell lung cancer tissue.

FIG. 17 shows evidence that anti-Trop2 and anti-GCC ADCs of the present invention are capable of simulating macrophages in the vicinity of antigen-expressing pancreatic cancer tissue.

### DETAILED DESCRIPTION OF THE INVENTION

Several highly potent TLR agonists have been attached to antibody-directed tumor cells. The antibody-drug-conjugate (ADC) gets internalized into tumor tissue, releasing the drug. The drug permeates to nearby tissues resulting in immunoactivation.

Anti-tumor effects will be driven by localized release of the TLR-agonist inducing an adaptive immune response against the tumor. Anti-pathogen effects can be driven by attachment of the TLR agonist to an antibody that binds to the pathogen. The opsonized pathogen will then be taken up by dendritic cells (antigen-presenting cells) where the TLR agonist will be released—resulting in an enhanced adaptive immune response.

In some embodiments, the compound is a compound of Formula (I):

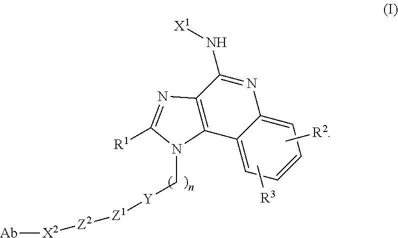

In some embodiments, the compound is a compound of Formula (II):

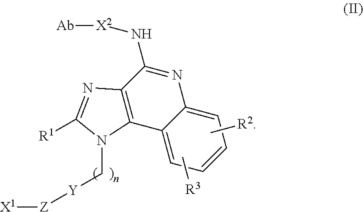

In some embodiments, the compound is a compound of Formula (III):

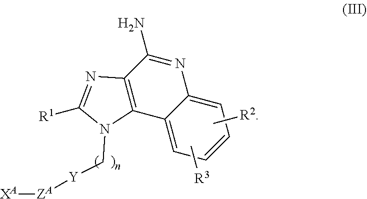

In some embodiments, R¹ is C₁-C₁₀ alkyl. In other embodiments, R¹ is n-butyl. In some embodiments, R¹ is C₁-C₁₀ oxaalkyl. In other embodiments, R¹ is —CH₂OH. In still other embodiments, R¹ is —CH₂CH₂OH, or R¹ is —CH₂CH₂CH₂OH. In some embodiments, R¹ is —CH₂OCH₂CH₃. In other embodiments, R¹ is —CH₂OCH₂CH₂CH₃, or R¹ is —CH₂CH₂OCH₂CH₃, or R¹ is —CH₂CH₂OCH₃. In still other embodiments, R¹ is C₁-C₁₀ azaalkyl. In some embodiments, R¹ is —CH₂NHCH₂CH₃. In other embodiments, R¹ is —CH₂NHCH₂CH₂CH₃, or R¹ is —CH₂CH₂NHCH₂CH₃, or R¹ is-CH₂CH₂NHCH₃.

In some embodiments, n is 1. In some embodiments, n is 2.

In some embodiments, Y is unsubstituted or substituted aryl. In other embodiments, Y is unsubstituted or substituted phenyl. In some embodiments, Y is unsubstituted or substituted heteroaryl. In other embodiments, Y is unsubstituted or substituted pyridyl. In some embodiments, Y is substituted with one or more of halogen, C₁-C₄ alkyl, C₁-C₄ alkoxy, C₁-C₄ haloalkyl, and/or C₁-C₄ haloalkoxy. In other embodiments, Y is substituted with one or more of chloro, fluoro, methyl, ethyl, propyl, and/or methoxy.

In some embodiments, Z¹ is —NR^{Z}—. In other embodiments, Z¹ is —O—. In some embodiments, Z¹ is —NR^{Z}C(O)—. In some embodiments, Z¹ is —NR^{Z}C(O)—O—. In still other embodiments, Z¹ is —NR^{Z}SO₂—. In some embodiments, Z¹ is —NR^{Z}— or —O—.

In some embodiments, Z² is absent. In other embodiments, Z² is —(C₁-C₅)hydrocarbon-NH—. In still other embodiments, Z² is —(C₁-C₅)alkyl-NH—. In other embodiments, Z² is -benzyl-NH—. In yet other embodiments, Z² is -phenyl-NH—. In some embodiments, Z² is a 5- to 8-membered nitrogen-containing heterocycle, wherein a nitrogen of the heterocycle is attached to X².

In some embodiments, Z is —O—. In other embodiments, Z is —NR^{Z}—. In still other embodiments, Z is —NR^{Z}C(O)—.

In some embodiments, Z^{A} is —NR^{Z}—. In some embodiments, Z^{A} is —NR^{Z}C(O)—. In some embodiments, Z^{A} is —NR^{Z}C(O)—O—. In other embodiments, Z^{A} is —NR^{Z}C(O)—(CH₂)ₖ—NH—. In some embodiments, Z^{A} is —NR^{Z}C(O)—(CH₂)ₖ—O—. In some embodiments, Z^{A} is —NR^{Z}C(O)—O—(CH₂)ₖ—O—. In other embodiments, Z^{A} is —NR^{Z}C(O)—(CH₂)ₖ—N(CH₃)—. In still other embodiments, Z^{A} is —NR^{Z}C(O)—O—(CH₂)ₖ—NH—. In some embodiments, Z^{A} is —NR^{Z}C(O)—(CH₂)ₖ—NH—C(O)—O—. In some embodiments, Z^{A} is —NR^{Z}SO₂—.

In some embodiments, X¹ is R^{Z}. In some embodiments, X¹ is hydrogen. In some embodiments, X¹ is methyl. In other embodiments, X¹ is C(O)—R^{Z}. In still other embodiments, X¹ is C(O)—O—R^{Z}. In some embodiments, X¹ is C(O)—N—(R^{Z})₂. In other embodiments, X¹ is SO₂—R^{Z}. In other embodiments, X¹ is —(CH₂)ₖNR^{Z}C(O)—(C₁-C₆)alkyl. In yet other embodiments, X¹ is —(CH₂)ₖNR^{Z}C(O)—O—(C₁-C₄)alkyl.

In some embodiments, R^{Z} is hydrogen. In other embodiments, R^{Z} is C₁-C₈ hydrocarbon. In still other embodiments, R^{Z} is C₁-C₈ alkyl. In some embodiments, R^{Z} is methyl. In other embodiments, R^{Z} is C₁-C₈ oxaalkyl. In still other embodiments, R^{Z} is C₁-C₈ azaalkyl. In yet other embodiments, R^{Z} is —C(NH₂)benzyl. In some embodiments, R^{Z} is heteroaryl. In some embodiments, R^{Z} is a 5- to 8-membered heterocyclic ring. Each instance of R^{Z} is independently selected. As non-limiting illustrative examples, if Z is —NR^{Z}— and X¹ is —C(O)—R^{Z}, both instances of R^{Z} may be hydrogen, or Z may be —NCH₂— and X¹ may be —C(O)CH₃, or Z may be —NH— and X¹ may be —C(O)CH₂CH₃.

In some embodiments, k is an integer from 1 to 8. In some embodiments, k is an integer from 1 to 6. In some embodiments, k is an integer from 1 to 4. In some embodiments, k is an integer from 1 to 3. In some embodiments, k is an integer from 1 to 2. In some embodiments, k is an integer from 2 to 4. In some embodiments, k is an integer from 2 to 3.

In some embodiments, k is 1. In some embodiments, k is 2. In some embodiments, k is 3. In some embodiments, k is 4. In some embodiments, k is 5. In some embodiments, k is 6. In some embodiments, k is 7. In some embodiments, k is 8.

In some embodiments, X^{A} is hydrogen. In some embodiments, X^{A} is C₁-C₁₀ alkyl. In some embodiments, X^{A} is C₁-C₄ alkyl. In some embodiments, X^{A} is methyl. In some embodiments, X^{A} is ethyl. In some embodiments, X^{A} is propyl. In some embodiments, X^{A} is butyl. In some embodiments, X^{A} is n-butyl. In some embodiments, X^{A} is t-butyl. In some embodiments, X^{A} is —C(O)CH₃.

In some embodiments when the compound is of Formula (I), X¹ is hydrogen, n is 1, and Y is phenyl, the compound is of formulae (Ia), (Ib), (Ic), (Id), (le), (If), (Ig), or (Ih):

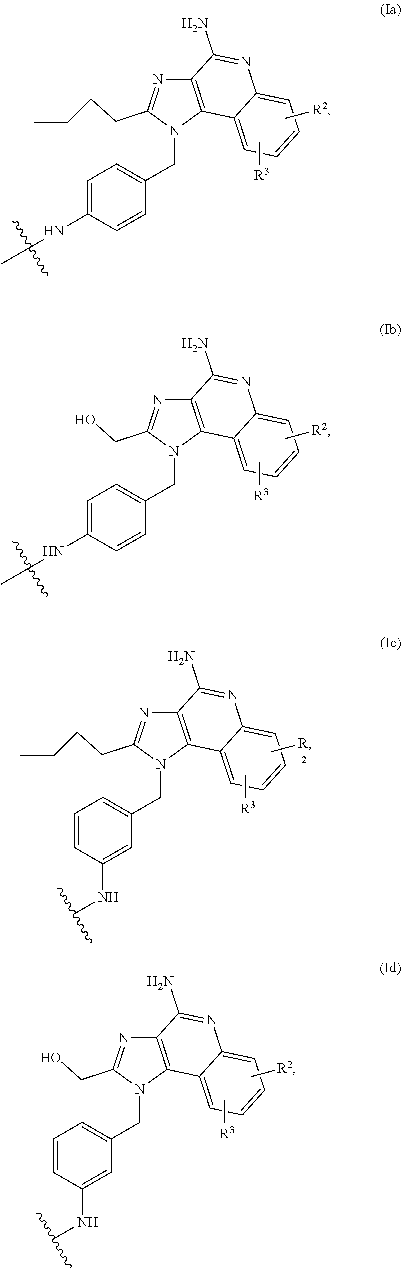

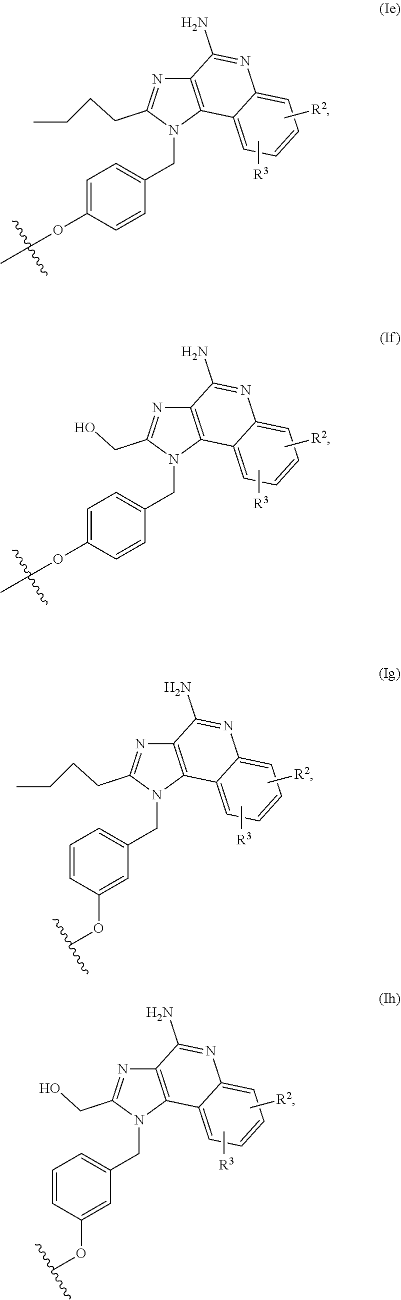

In these embodiments of formulae (Ia), (Ib), (Ic), (Id), (Ie), (If), (Ig), or (Ih), [symbol] represents a point of attachment to X₂.

In some embodiments when the compound is of Formula (II), n is 1, and Y is phenyl, the compound is of formulae (IIa), (IIb), (IIc), (IId), (IIe), (IIf), (IIg), or (IIh):

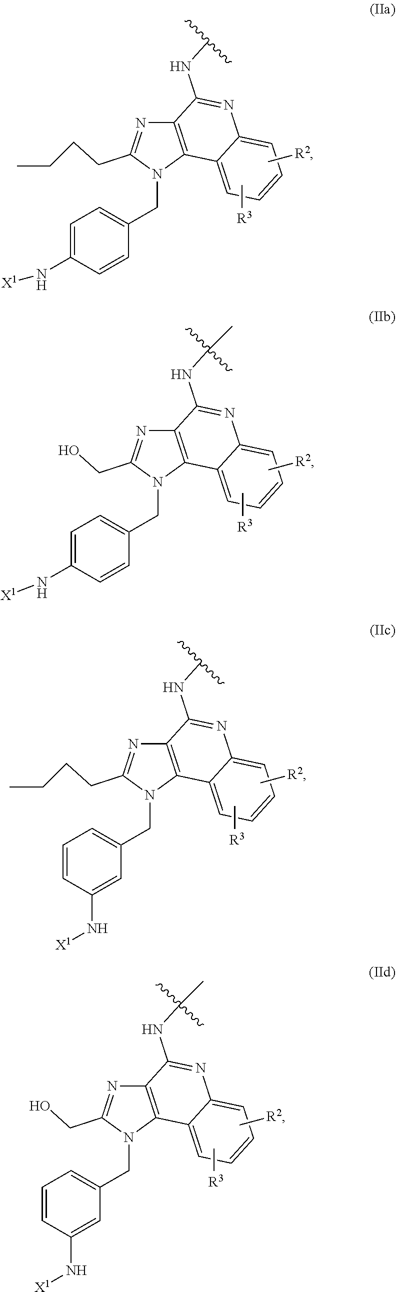

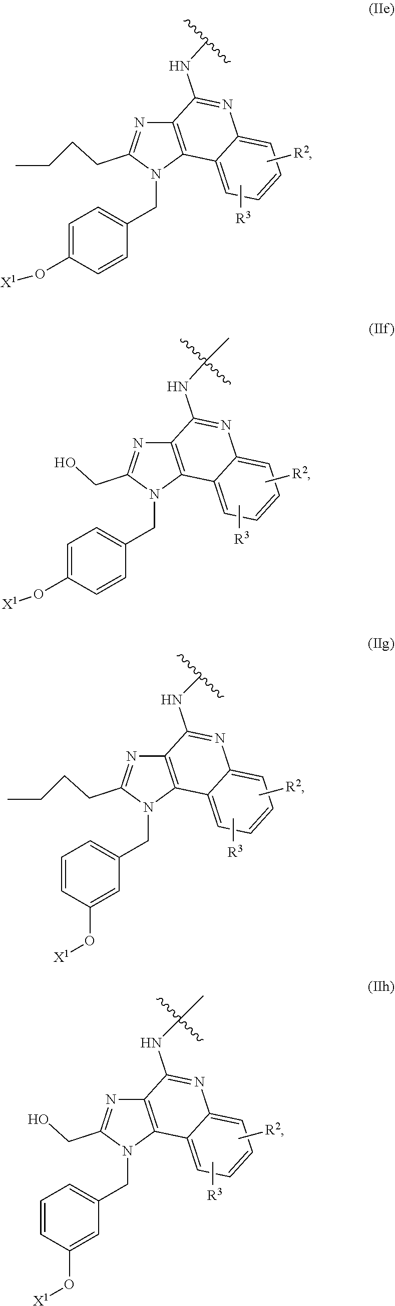

wherein [symbol] represents a point of attachment to X₂.

In some embodiments, R² is hydrogen. In other embodiments, R² is C₁-C₅ alkyl. In some embodiments, R² is selected from methyl, ethyl, propyl, or butyl. In still other embodiments, R² is C₁-C₅ alkoxy. In some embodiments, R² is selected from methoxy, ethoxy, propoxy, or butoxy.

In some embodiments, R³ is hydrogen. In other embodiments, R³ is C₁-C₅ alkyl. In some embodiments, R³ is selected from methyl, ethyl, propyl, or butyl. In still other embodiments, R³ is C₁-C₅ alkoxy. In some embodiments, R² is selected from methoxy, ethoxy, propoxy, or butoxy.

To be abundantly clear, R² and R³ are independently selected. As non-limiting examples, both R² and R³ may be hydrogen, both R² and R³ may be a C₁-C₅ alkyl, or one of R² and R³ may be C₁-C₅ alkoxy and the other of R² and R³ may be hydrogen.

In one embodiment, X² is L1-L2-(L3)ₚ-(L4)_{q}-(L5)ᵣ.

In one embodiment, the X² of the compound of Formula (I) or Formula (II) is a cleavable or noncleavable linker (referred to herein as “linker” or “L” as further described herein).

The linker may be cleavable, consisting of a chemically labile linker including acid-cleavable linkers and reducible linkers or an enzyme cleavable linker such as peptide-based linkers or glucuronide linkers well known in the art. In one embodiment, the linker is cleavable via intracellular enzymes (e.g., cathepsin-B or Legumain).

In an ADC the linker serves to attach the payload to the antibody.

In one embodiment, a second section of the linker unit is introduced which has a second reactive site e.g., an electrophilic group that is reactive to a nucleophilic group present on an antibody unit (e.g., an antibody). Useful nucleophilic groups on an antibody include but are not limited to, sulfhydryl, hydroxyl, and amino groups. The heteroatom of the nucleophilic group of an antibody may be reactive to an electrophilic group on a linker unit and forms a covalent bond to a linker unit. Useful electrophilic groups include, but are not limited to, maleimide, haloacetamide, and activated ester groups. The electrophilic group may provide a convenient site for antibody attachment.

In another embodiment, a linker unit has a reactive site which has a nucleophilic group that is reactive to an electrophilic group present on an antibody. Useful electrophilic groups on an antibody include, but are not limited to, aldehyde and ketone carbonyl groups.

The heteroatom of a nucleophilic group of a linker unit can react with an electrophilic group on an antibody and form a covalent bond to the antibody. Useful nucleophilic groups on a linker unit include, but are not limited to, hydrazide, oxime, amino, hydrazine, thiosemicarbazone, hydrazine carboxylate, and arylhydrazide. The electrophilic group on an antibody may provide a convenient site for attachment to a linker unit. In another embodiment, a linker unit has a functionality that can be attached to the antibody through an enzymatic reaction. One particularly useful example of this is the transamidation of amine-containing linkers with glutamine, a reaction that is promoted by bacterial transglutaminase.

This reaction can be used to attach payloads to endogenous glutamine residues, as in Benjamin et al. (Mol. Pharmaceutics 2019, 16, 6, 2795-2807) or may be used to attach payloads to specifically engineered glutamine tags, as in Strop et al. (Chemistry and Biology 2013, 20, 2, 161-167), both of which are hereby incorporated by reference in their entirety

Amino functional groups are also useful reactive sites for a linker unit because they can react with carboxylic acid, or activated esters of a compound to form an amide linkage. The peptide-based compounds of the present disclosure may, in one embodiment, be prepared by forming a peptide bond between two or more amino acids and/or peptide fragments. Such peptide bonds can be prepared, for example, according to the liquid phase synthesis method (see, e.g., Schroder and Lubke, THE PEPTIDES, 1^{st} Ed., pp 76-136 (Academic Press 1966), which is hereby incorporated by reference in its entirety) that is well known in the field of peptide chemistry.

In the context of the present disclosure, particularly but not limited to linker components, the language “selected from one or more of” or “one or more of” indicates that multiple components, which may be the same or different, are or may be arranged sequentially. Thus, for example, L2 may be any individually or combined listed components.

In accordance with the present disclosure, the linker of Formula (I) or Formula (II) is defined as X² and, in some embodiments, X² is L1-L2-(L3)ₚ-(L4)_{q}-(L5)ᵣ. In accordance with one embodiment of the present disclosure, L1 is a conjugation moiety. A conjugation moiety as described herein includes a moiety that attaches L2 as described herein to a cysteine, lysine, or glutamine residue. In some embodiments, the glutamine is glutamine 295. Examples of L1 include maleimide, bromoacetamide, amine, NHS-ester, and the like.

In one embodiment, L2 as described herein is a spacer unit selected from branched or unbranched C₁-C₁₂ alkyl, a PEG selected from PEG1 to PEG12,

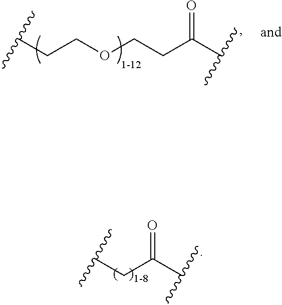

PEG may, for example, be PEG1, PEG2, PEG3, PEG4, PEG5, PEG6, PEG7, PEG8, PEG9, PEG10, PEG11, PEG12, or any combination thereof.

In one embodiment, L3 as described herein relates to a peptide of 1 to 6 amino acids. For example, the peptide may be 1 amino acid, 2 amino acids, 3 amino acids, 4 amino acids, 5 amino acids, or 6 amino acids. Amino acids may be selected both from natural amino acids and non-natural α-amino acids.

In one embodiment, L4 is a self-immolative spacer.

In one embodiment, L5 is carbonyl, as described herein.

In one embodiment, p, q, and r of the compound of Formula (I) and Formula (II) are each independently selected from 0 and 1.

In one embodiment, the compound of Formula (I) or Formula (II) may include L1 selected from:

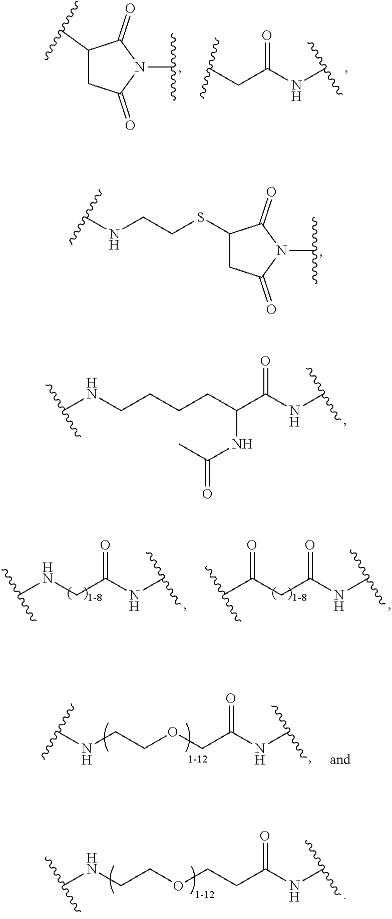

In another embodiment, the compound of Formula (I) or Formula (II) may include L2 selected from

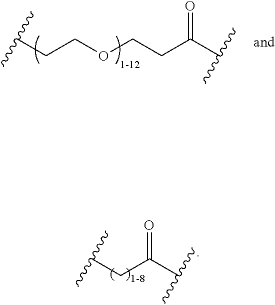

In yet another embodiment, the compound of Formula (I) or Formula (II) may include L3 of

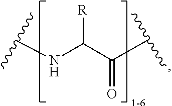

wherein R is an amino acid side chain. In yet another embodiment, the compound of Formula (I) or Formula (II) may include L4 selected from:

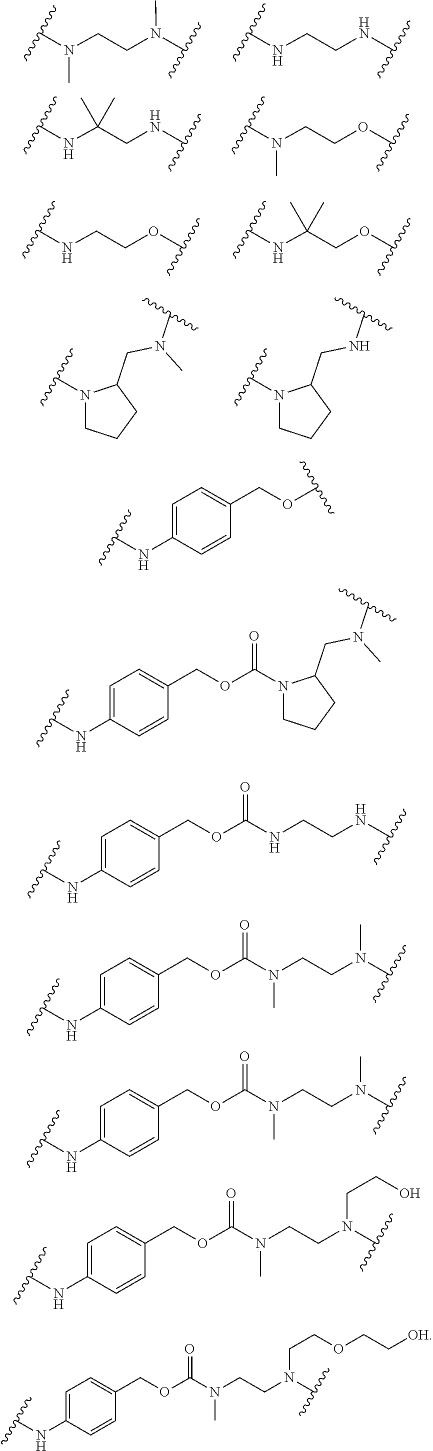

In yet another embodiment, the compound of Formula (I) or Formula (II) may include L5 of

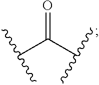 [SMILES: *C(*)=O]

and p, q, and r are each independently 0 or 1, wherein when q is 0 then r is 0, or when p is 1 then r is 1.

L3 may, in certain embodiments, include, but is not limited to, ValCit, GlyValCit, ValArg, PheLys, AlaAla, GlyGlyPheGly, AlaAlaAla, AlaAsn, AsnAsn, AsnAla, ValCitGlyPro, AsnGlyPro, AsnAsnGlyPro, Asn, GlyAsn, AsnAla, ProCitAla, ProAsnLeu, ProAsnAla, ProPheAla, ProPheGly, ProCitLeu, ProAsnPro, ProAsnSer, and ProAsnGly.

In one embodiment, the compound of Formula (I) or Formula (II) may include L1 that is selected from:

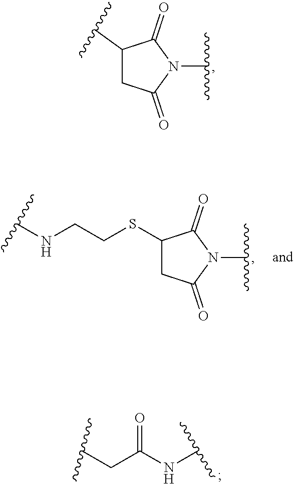 [SMILES: *C1CC(=O)N(*)C1=O ; *NCCSC1CC(=O)N(*)C1=O ; *CC(=O)N*]

L2 that is

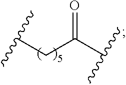

L3 that is ValCit, GlyValCit, AsnAsn, Asn or AlaAla; L4 that is selected from:

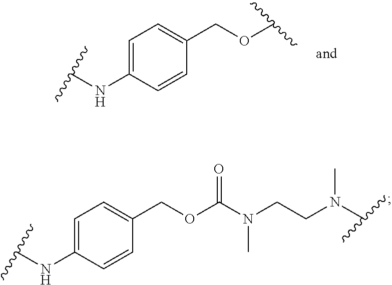 [SMILES: *Nc1ccc(CO*)cc1 ; *Nc1ccc(COC(=O)N(C)CCN(*)C)cc1]

and/or L5 that is

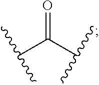 [SMILES: *C(*)=O]

and p, q, and r are each 0 or p, q, and r are each 1.

In the compounds described herein, X² may, in one embodiment, be attached to Ab through a cysteine residue of Ab, a lysine residue of Ab, or a glutamine residue of Ab. In some embodiments, the glutamine is glutamine 295.

In some embodiments, X² is attached to Ab through a glutamine residue of Ab, wherein the glutamine is glutamine 295, and L1 is

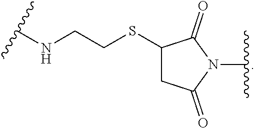 [SMILES: *NCCSC1CC(=O)N(*)C1=O]

Unless explicitly specified, compounds of Formula (I) or Formula (II) or Formula (III) include compounds of formulae (Ia), (Ib), (Ic), (Id), (Ie), (If), (Ig), (Ih), or (IIa), (IIb), (IIc), (IId), (IIe), (IIf), (IIg), (IIh), or (III). Unless explicitly specified, the embodiments described herein relate to any of Formula (I) or Formula (II) or Formula (III), or formulae (Ia), (Ib), (Ic), (Id), (Ie), (If), (Ig), (Ih), (IIa), (IIb), (IIc), (IId), (IIe), (IIf), (IIg), (IIh), or (III).

In one aspect, the present invention provides a pharmaceutical composition comprising a compound described herein and a pharmaceutically acceptable carrier, diluent, or excipient. In one embodiment, the pharmaceutical composition further comprises a therapeutically effective amount of a chemotherapeutic agent.

In one aspect, the present invention provides a method for stimulating an immune response in a subject. The method includes administering a therapeutically effective amount of a compound described herein under conditions effective to stimulate an immune response.

In some embodiments, the method is performed on a subject having cancer. In other embodiments, the cancer is bladder cancer, breast cancer, cervical cancer, colon cancer, endometrial cancer, kidney cancer, lung cancer, esophageal cancer, ovarian cancer, prostate cancer, pancreatic cancer, skin cancer, gastric cancer, testicular cancer, biliary cancer, colorectal cancer, endometrial cancer, head/neck cancer, medullary thyroid cancer, renal cancer, eye cancer, neuroblastoma, Mycosis fungoides, glial tumor, other brain tumor, spinal cord tumor, liver cancer, leukemia, lymphoma, or any combination thereof.

In certain embodiments, the immunotherapy compounds present in a liquid pharmaceutical composition are administered into a tumor (e.g., intratumoral (IT) administration) and induce an innate immune response and a cell-mediated immune response against the tumor antigens (e.g., shrink or stabilize the tumor). The conjugate comprising a peptide is not necessarily an antigen or immunogen, but a mechanism to reduce the solubility of the TLR7 and/or TLR8 agonist creating a depot that is retained at the site of administration, such as within a tumor or in the tumor microenvironment. The conjugated TLR7 and/or TLR8 agonist may stimulate immunosuppressive cells and may induce the immune response against the antigens present in the tumor. Moreover, mobilization of the immunosuppressive cells may induce an immune response against not only the tumor at the site of administration, but peripheral, nearby and/or distant tumors as well. In one embodiment, methods of stimulating an anti-tumor immune response in a subject are disclosed, where the methods comprise locally administering intratumorally or peritumorally a liquid form of the pharmaceutical composition into the subject, where the anti-tumor immune response is effective at a distant site from the site of administration of the pharmaceutical composition.

The present invention provides, in a fourth aspect, a method for inducing an anti-tumor immune response in a subject. The method includes administering a therapeutically effective amount of a compound described herein under conditions effective to induce an anti-tumor immune response. In some embodiments, the method is performed on a selected subject having a tumor. In some embodiments, the tumor is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, or retinoblastoma.

The present invention provides, in a fifth aspect, a method for treating a tumor or abnormal cell proliferation in a subject. The method includes administering a therapeutically effective amount of a compound described herein under conditions effective to treat a tumor or abnormal cell proliferation. In some embodiments, the tumor or abnormal cell proliferation is cancer. In some embodiments, the cancer is bladder cancer, breast cancer, cervical cancer, colon cancer, endometrial cancer, kidney cancer, lung cancer, esophageal cancer, ovarian cancer, prostate cancer, pancreatic cancer, skin cancer, gastric cancer, testicular cancer, biliary cancer, colorectal cancer, endometrial cancer, head and neck cancer, medullary thyroid cancer, renal cancer, eye cancer, neuroblastoma, Mycosis fungoides, glial tumor, other brain tumor, spinal cord tumor, liver cancer, leukemia, lymphoma, or any combination thereof.

The present invention provides, in a sixth aspect, a method for treating an infectious disease in a subject. The method includes administering a therapeutically effective amount of a compound described herein under conditions effective to treat an infectious disease. In some embodiments, the infectious disease is a viral infection, a bacterial infection, a fungal infection, or any combination thereof. In some embodiments, the infectious disease is a viral infection, and the infectious disease is a coronavirus (including, but not limited to, Severe Acute Respiratory Syndrome (SARS), SARS-CoV-2 (COVID-19), Middle East Respiratory Syndrome (MERS), and the common cold), Ebola, influenza, hepatitis, Hib disease, human immunodeficiency virus (HIV), human papillomavirus (HPV), meningococcal disease, pneumococcal disease, measles, mumps, norovirus, polio, respiratory syncytial virus (RSV), rotavirus, rubella virus, shingles, West Nile virus, rabies virus, enterovirus, cytomegalovirus, herpes virus, varicella, Yellow fever, Zika virus, or any combination thereof. In some embodiments, the infectious disease is a bacterial infection, and the infectious disease is selected from streptococcal disease, staphylococcal disease, diphtheria, meningococcal disease, tetanus, pertussis, pneumococcal disease, bacterial food poisoning, a sexually transmitted infection, tuberculosis, Lyme disease, botulism, or any combination thereof. In some embodiments, the infectious disease is a fungal infection, and the infectious disease is candidiasis, histoplasmosis, dermatophytosis, tinea pedis, aspergillosis, cryptococcal meningitis, coccidioidomycosis, or any combination thereof.

### Abbreviations and Definitions

Unless defined otherwise, all technical and scientific terms used herein have the same meaning as is commonly understood by one of ordinary skill in the art to which this disclosure belongs. A comprehensive list of abbreviations utilized by organic chemists (i.e., persons of ordinary skill in the art) appears in the first issue of each volume of the Journal of Organic Chemistry. The list, which is typically presented in a table entitled “Standard List of Abbreviations” is incorporated herein by reference. In the event that there is a plurality of definitions for terms cited herein, those in this section prevail unless otherwise stated.

The following abbreviations and terms have the indicated meanings throughout:
Ac=acetyl
Aq=aqueous
Boc=t-butyloxy carbonyl
Bu=butyl
c-=cyclo
DCM=dichloromethane=methylene chloride=CH₂Cl₂
DMA=dimethylacetamide
DMF=N,N-dimethylformamide
eq. or equiv.=equivalent(s)
Et=ethyl
h=hour(s)
HATU=hexafluorophosphate azabenzotriazole tetramethyl uronium
HOBt=hydroxybenzotriazole
me=maleimidocaproyl
mCPBA=meta-Chloroperoxybenzoic acid
Me=methyl
min.=minute(s)
PAB=4-aminobenzyl
PABC=p-aminobenzylcarbamate
Pg=protecting group
Ph=phenyl
PNP=p-nitrophenol
RT=room temperature
sat'd or sat.=saturated
SEAP=secreted embryonic alkaline phosphatase
STD=standard deviation
t- or tert=tertiary
TFA=trifluoroacetic acid
THE=tetrahydrofuran
Tosyl=p-toluenesulfonyl
UPLC=ultra performance liquid chromatography

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof, but only if the additional features, integers, steps, components or groups thereof do not materially alter the basic and novel characteristics of the claimed composition or method.

For purposes of the present disclosure, the term “antibody” (or “Ab” or “AB”) herein is used in the broadest sense and specifically covers intact monoclonal antibodies, polyclonal antibodies, monospecific antibodies, multispecific antibodies (e.g., bispecific antibodies), antibody fragments that exhibit desired biological activity, genetically engineered forms of the antibodies, and combinations thereof. In addition, while certain aspects of the present disclosure refer to antibody drug conjugates, it is envisioned that the antibody portion of the conjugate may be replaced with anything that specifically binds or reactively associates or complexes with a receptor, antigen, or other receptive moiety associated with a given target-cell population. For example, conjugates of the present disclosure could include a targeting molecule that binds to, complexes with, or reacts with a receptor, antigen, or other receptive moiety of a cell population sought to be therapeutically or otherwise biologically modified. Examples of such molecules include small molecular weight proteins, polypeptide or peptides, lectins, glycoproteins, non-peptides, vitamins, nutrient-transport molecules (for example, transferrin), or any other cell binding molecule or substances. In certain aspects, the antibody or other such targeting molecule acts to deliver a drug to the particular target cell population with which the antibody or other targeting molecule interacts. In one embodiment, “Ab” comprises an antibody or an antibody fragment. While some specific examples of antibodies (i.e., “Ab”) are disclosed herein, antibodies that can successfully be used are not limited to these examples, as the person of skill will understand.

The term “antibody,” which is used interchangeably with the term “immunoglobulin,” includes full length (i.e., naturally occurring or formed by normal immunoglobulin gene fragment recombinatorial processes) immunoglobulin molecules (e.g., an IgG antibody) and immunologically active fragments thereof (i.e., including the specific binding portion of the full-length immunoglobulin molecule), which again may be naturally occurring or synthetic in nature. Accordingly, the term “antibody fragment” includes a portion of an antibody such as F(ab′)₂, F(ab)₂, Fab′, Fab, Fv, scFv and the like. Regardless of structure, an antibody fragment binds with the same antigen that is recognized by the full-length antibody. Methods of making and screening antibody fragments are well-known in the art.

Naturally occurring antibodies typically have two identical heavy chains and two identical light chains, with each light chain covalently linked to a heavy chain by an inter-chain disulfide bond and multiple disulfide bonds further link the two heavy chains to one another. Individual chains may fold into domains having similar sizes (110-125 amino acids) and structures, but different functions. The light chain can comprise one variable domain (V_{L}) and/or one constant domain (CL). The heavy chain can also comprise one variable domain (V_{H}) and/or, depending on the class or isotype of antibody, three or four constant domains (CH1, CH2, CH3, and CH4). The variable region binds to and interacts with a target antigen. The variable region includes a complementary determining region (CDR) that recognizes and binds to a specific binding site on a particular antigen. The constant region may be recognized by and interact with the immune system (see, e.g., Janeway et al., IMMUNOBIOLOGY, 5th Ed., Garland Science (New York 2001), which is hereby incorporated by reference in its entirety). An antibody can be of any type or class (e.g., IgG, IgE, IgM, IgD, and IgA) or subclass (e.g., IgG1, IgG2, IgG3, IgG4, IgA1, and IgA2). In humans, the isotypes are IgA, IgD, IgE, IgG, and IgM, with IgA and IgG further subdivided into subclasses or subtypes (IgA1-2 and IgG1-4). The antibody can be derived from any suitable species. In some embodiments, the antibody is of human or murine origin. An antibody can be, for example, human, humanized or chimeric.

Generally, the variable domains show considerable amino acid sequence variability from one antibody to the next, particularly at the location of the antigen-binding site. Three regions, called hyper-variable or complementarity-determining regions (CDRs), are found in each of V_{L} and V_{H}, which are supported by less variable regions called framework variable regions. Antibodies include IgG monoclonal antibodies as well as antibody fragments or engineered forms. These are, for example, Fv fragments, or proteins wherein the CDRs and/or variable domains of the exemplified antibodies are engineered as single-chain antigen-binding proteins.

The portion of an antibody consisting of the V_{L} and V_{H} domains is designated as an Fv (Fragment variable) and constitutes the antigen-binding site. A single chain Fv (scFv or SCA) is an antibody fragment containing a V_{L} domain and a V_{H} domain on one polypeptide chain, wherein the N terminus of one domain and the C terminus of the other domain are joined by a flexible linker. The peptide linkers used to produce the single chain antibodies are typically flexible peptides, selected to assure that the proper three-dimensional folding of the V_{L} and V_{H} domains occurs. The linker is generally 10 to 50 amino acid residues, and in some cases is shorter, e.g., about 10 to 30 amino acid residues, or 12 to 30 amino acid residues, or even 15 to 25 amino acid residues. An example of such linker peptides includes repeats of four glycine residues followed by a serine residue.

Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Fab (Fragment, antigen binding) refers to the fragments of the antibody consisting of the V_{L}, CL, V_{H}, and CH1 domains. Those generated following papain digestion simply are referred to as Fab and do not retain the heavy chain hinge region. Following pepsin digestion, various Fabs retaining the heavy chain hinge are generated. Those fragments with the interchain disulfide bonds intact are referred to as F(ab′)2, while a single Fab′ results when the disulfide bonds are not retained. F(ab′)₂ fragments have higher avidity for antigen that the monovalent Fab fragments.

Fc (Fragment crystallization) is the designation for the portion or fragment of an antibody that comprises paired heavy chain constant domains. In an IgG antibody, for example, the Fc comprises CH2 and CH3 domains. The Fc of an IgA or an IgM antibody further comprises a CH4 domain. The Fc is associated with Fc receptor binding, activation of complement mediated cytotoxicity and antibody-dependent cellular-cytotoxicity (ADCC). For antibodies such as IgA and IgM, which are complexes of multiple IgG-like proteins, complex formation requires Fc constant domains.

Finally, the hinge region separates the Fab and Fc portions of the antibody, providing for mobility of Fabs relative to each other and relative to Fc, as well as including multiple disulfide bonds for covalent linkage of the two heavy chains.

Antibody “specificity” refers to selective recognition of an antibody for a particular epitope of an antigen. The term “epitope” includes any protein determinant capable of specific binding to an immunoglobulin or T-cell receptor or otherwise interacting with a molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between the protein and the interacting molecule (such as an antibody) occur linearly along the primary amino acid sequence of the protein. In a conformational epitope, the points of interaction occur across amino acid residues on the protein that are separated from one another, i.e., noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen. As described herein, the phrases “specifically binds” and “specific binding” refer to antibody binding to a predetermined antigen.

Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. The modifier “monoclonal” indicates the character of the antibody as being obtained from a substantially homogeneous population of antibodies, and is not to be construed as requiring production of the antibody by any particular method.

Monoclonal antibodies may be murine, human, humanized, or chimeric. A humanized antibody is a recombinant protein in which the CDRs of an antibody from one species; e.g., a rodent, rabbit, dog, goat, horse, or chicken antibody (or any other suitable animal antibody), are transferred into human heavy and light variable domains. The constant domains of an antibody molecule are derived from those of a human antibody. Methods for making humanized antibodies are well known in the art. Chimeric antibodies preferably have constant regions derived substantially or exclusively from human antibody constant regions and variable regions derived substantially or exclusively from the sequence of the variable region from a mammal other than a human. The chimerization process can be made more effective by also replacing the variable regions—other than the hyper-variable regions or the complementarity—determining regions (CDRs), of a murine (or other non-human mammalian) antibody with the corresponding human sequences. The variable regions other than the CDRs are also known as the variable framework regions (FRs).

The term “monoclonal antibodies” specifically includes “chimeric” antibodies in which a portion of the heavy and/or light chain is identical to or homologous with the corresponding sequence of antibodies derived from a particular species or belonging to a particular antibody class or subclass, while the remainder of the chain(s) is identical to or homologous with the corresponding sequences of antibodies derived from another species or belonging to another antibody class or subclass, as well as fragments of such antibodies, so long as they exhibit the desired biological activity.

Useful monoclonal antibodies include, but are not limited to, human monoclonal antibodies, humanized monoclonal antibodies, antibody fragments, or chimeric monoclonal antibodies. Human monoclonal antibodies may be made by any of numerous techniques known in the art (e.g., Teng et al., “Construction and Testing of Mouse-Human Heteromyelomas for Human Monoclonal Antibody Production,” Proc. Natl. Acad. Sci. USA 80:7308-12 (1983); Kozbor et al., “The Production of Monoclonal Antibodies From Human Lymphocytes,” Immunology Today 4:72-79 (1983); and Olsson et al., “Human-Human Monoclonal Antibody-Producing Hybridomas: Technical Aspects,” Meth. Enzymol. 92:3-16 (1982), all of which are hereby incorporated by reference in their entirety).

The antibody can also be a bispecific antibody. Methods for making bispecific antibodies are known in the art and are discussed herein.

An “intact antibody” as described herein includes one which comprises an antigen-binding variable region as well as a light chain constant domain (CL) and heavy chain constant domains, Cm, CH2, Cm and CH4, as appropriate for the antibody class. The constant domains may be native sequence constant domains (e.g., human native sequence constant domains) or amino acid sequence variants thereof.

An intact antibody may have one or more “effector functions”, which refers to those biological activities attributable to the Fc region (e.g., a native sequence Fc region or amino acid sequence variant Fc region) of an antibody. Examples of antibody effector functions include complement dependent cytotoxicity, antibody-dependent cell-mediated cytotoxicity (ADCC) and antibody-dependent cell-mediated phagocytosis. See WO 2014/068443 to Pfizer Inc., which is hereby incorporated by reference in its entirety.

An “antibody fragment” comprises a portion of an intact antibody, preferably comprising the antigen-binding or variable region thereof. The antibody can be a functionally active fragment, derivative or analog of an antibody that immunospecifically binds to target cells (e.g., cancer cell antigens, viral antigens, or microbial antigens) or other antibodies that bind to tumor cells or matrix. In this regard, “functionally active” means that the fragment, derivative or analog is able to elicit anti-anti-idiotype antibodies that recognize the same antigen that the antibody from which the fragment, derivative or analog is derived recognized. Specifically, in an exemplary embodiment the antigenicity of the idiotype of the immunoglobulin molecule can be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art (e.g., the BIA core assay) (for location of the CDR sequences, see, e.g., Kabat et al., SEQUENCES OF PROTEINS OF IMMUNOLOGICAL INTEREST, Fifth Edition, National Institute of Health (Bethesda, Md. 1991); Kabat E., “Origins of Antibody Complementarity and Specificity-Hypervariable Regions and Minigene Hypothesis,” J. Immunology 125(3):961-969) (1980), both of which are hereby incorporated by reference in their entirety).

Examples of antibody fragments include Fab, Fab′, F(ab′)2, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, any other molecule with the same specificity as the antibody, or an epitope-binding fragments of any of the above which immunospecifically bind to a target antigen (e.g., a cancer cell antigen, a viral antigen or a microbial antigen).

The term “variable” in the context of an antibody refers to certain portions of the variable domains of the antibody that differ extensively in sequence and are used in the binding and specificity of each particular antibody for its particular antigen. This variability is concentrated in three segments called “hypervariable regions” in the light chain and the heavy chain variable domains. The more highly conserved portions of variable domains are called the framework regions (FRs). The variable domains of native heavy and light chains each comprise four FRs connected by three hypervariable regions.

The phrase “hypervariable region” as used herein includes the amino acid residues of an antibody which are responsible for antigen-binding. The hypervariable region generally comprises amino acid residues from a “complementarity determining region” or “CDR” (e.g., residues 24-34 (LI), 50-56 (L2) and 89-97 (L3) in the light chain variable domain and 31-35 (H1), 50-65 (H2) and 95-102 (L3) in the heavy chain variable domain (Kabat et al., SEQUENCES OF PROTEINS OF IMMUNOLOGICAL INTEREST, Fifth Edition, National Institute of Health (Bethesda, Md. 1991), which is hereby incorporated by reference in its entirety); and/or those residues from a “hypervariable loop” (e.g., residues 26-32 (LI), 50-52 (L2) and 91-96 (L3) in the light chain variable domain and 26-32 (H1), 53-55 (142) and 96-101 (H3) in the heavy chain variable domain; Chothia and Lesk, “Canonical Structures For the Hypervariable Regions of Immunoglobulins,” J. Mol. Biol. 196:901-17 (1987), which is hereby incorporated by reference in its entirety). FR residues are those variable domain residues other than the hypervariable region residues as herein defined.

A “single-chain Fv” or “scFv” antibody fragment may include the V.sub.H and V.sub.L domains of an antibody, where these domains are present in a single polypeptide chain. Typically, the Fv polypeptide further comprises a polypeptide linker between the V.sub.H and V.sub.L domains which enables the scFv to form the desired structure for antigen binding. For a review of scFv, see Pluckthun in THE PHARMACOLOGY OF MONOCLONAL ANTIBODIES, vol. 113, Rosenburg and Moore eds., SpringerVerlag (New York 1994) pp. 269-315, which is hereby incorporated by reference in its entirety).

The term “diabody” includes small antibody fragments with two antigen-binding sites, which fragments comprise a variable heavy domain (V_{H}) connected to a variable light domain (V_{L}) in the same polypeptide chain. By using a linker that is too short to allow pairing between the two domains on the same chain, the domains are forced to pair with the complementary domains of another chain and create two antigen-binding sites. Diabodies are described more fully in, for example, EP 0 404 097 to BEHRINGWERKE AG; WO 93/11161 to Enzon, Inc.; and Hollinger et al., “‘Diabodies’: Small Bivalent and Bispecific Antibody Fragments,” Proc. Natl. Acad. Sci. USA 90:6444-6448 (1993), all of which are hereby incorporated by reference in their entirety.

Completely human antibodies are useful and can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of a polypeptide of the present disclosure. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, “Human Antibodies From Transgenic Mice,” Int. Rev. Immunol. 13:65-93 (1995), which is hereby incorporated by reference in its entirety. For a detailed discussion of this technology for producing human antibodies and human monoclonal antibodies and protocols for producing such antibodies, see, e.g., U.S. Pat. No. 5,625,126 to Lonberg et al.; U.S. Pat. No. 5,633,425 to Lonberg et al.; U.S. Pat. No. 5,569,825 to Lonberg et al.; U.S. Pat. No. 5,661,016 to Lonberg et al.; U.S. Pat. No. 5,545,806 to Lonberg et al., all of which are hereby incorporated by reference in their entirety.

Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope. See, e.g., Jespers et al., “Guiding the Selection of Human Antibodies From Phage Display Repertoires to a Single Epitope of an Antigen,” Biotechnology 12:899-903 (1994), which is hereby incorporated by reference in its entirety. Human antibodies can also be produced using various techniques known in the art, including phage display libraries (see, e.g., Hoogenboom and Winter, “By-Passing Immunisation. Human Antibodies From Synthetic Repertoires of Germline V_{H} Gene Segments Rearranged In Vitro,” J. Mol. Biol. 227:381 (1991); Marks et al., “By-Passing Immunization. Human Antibodies From V-gene Libraries Displayed on Phage,” J. Mol. Biol. 222:581 (1991); Quan and Carter, “The rise of monoclonal antibodies as therapeutics,” In ANTI-IGE AND ALLERGIC DISEASE, Jardieu and Fick, eds., Marcel Dekker (New York, N.Y., 2002) Chapter 20, pp. 427-469), all of which are hereby incorporated by reference in their entirety.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., “Replacing the Complementarity-Determining Regions in a Human Antibody With Those From a Mouse,” Nature 321:522-25 (1986); Riechmann et al., “Reshaping Human Antibodies For Therapy,” Nature 332:323-329 (1988); and Presta, L. “Antibody Engineering,” Curr. Op. Struct. Biol. 2:593-596 (1992), all of which are hereby incorporated by reference in their entirety.

Recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as for example, those having a variable region derived from a murine monoclonal and human immunoglobulin constant regions (see, e.g., U.S. Pat. No. 4,816,567 to Cabilly et al.; and U.S. Pat. No. 4,816,397 to Boss et al., which are incorporated herein by reference in their entirety). Humanized antibodies are antibody molecules from non-human species having one or more complementarity determining regions (CDRs) from the non-human species and a framework region from a human immunoglobulin molecule (see, e.g., U.S. Pat. No. 5,585,089 to Queen et al., which is incorporated herein by reference in its entirety). Such chimeric and humanized monoclonal antibodies can be produced by recombinant DNA techniques known in the art, for example using methods described in International Publication No. WO 87/02671 to Int Genetic Eng; European Patent Publication No. 0 184 187 to Teijin Ltd; European Patent Publication No. 0 171 496 to Japan Res Dev Corp; European Patent Publication No. 0 173 494 to Univ Leland Stanford Junior; International Publication No. WO 86/01533 to Celltech Ltd; U.S. Pat. No. 4,816,567 to Cabilly et al.; Berter et al., “Escherichia coli Secretion of an Active Chimeric Antibody Fragment,” Science 240:1041-1043 (1988); Liu et al., “Chimeric Mouse-Human IgG1 Antibody That Can Mediate Lysis of Cancer Cells,” Proc. Natl. Acad. Sci. USA 84:3439-3443 (1987); Liu et al., “Production of a Mouse-Human Chimeric Monoclonal Antibody to CD20 With Potent Fc-Dependent Biologic Activity,” J. Immunol. 139:3521-3526 (1987); Sun et al., “Chimeric Antibody With Human Constant Regions and Mouse Variable Regions Directed Against Carcinoma-Associated Antigen 17-1A,” Proc. Natl. Acad. Sci. USA 84:214-218 (1987); Nishimura et al., “Recombinant Human-Mouse Chimeric Monoclonal Antibody Specific for Common Acute Lymphocytic Leukemia Antigen,” Cancer. Res. 47:999-1005 (1987); Wood et al., “The Synthesis and In Vivo Assembly of Functional Antibodies in Yeast,” Nature 314:446-449 (1985); and Shaw et al., “Mouse/Human Chimeric Antibodies to a Tumor-Associated Antigen: Biologic Activity of the Four Human IgG Subclasses,” J. Natl. Cancer Inst. 80:1553-1559 (1988); Morrison, S. L., “Transfectomas Provide Novel Chimeric Antibodies,” Science 229:1202-1207 (1985); U.S. Pat. No. 5,225,539 to Winter; Jones et al., “Replacing the Complementarity-Determining Regions in a Human Antibody With Those From a Mouse,” Nature 321:552-525 (1986); Verhoeyan et al., “Reshaping Human Antibodies: Grafting an Antilysozyme Activity,” Science 239:1534 (1988); and Beidler et al., “Cloning and High Level Expression of a Chimeric Antibody With Specificity For Human Carcinoembryonic Antigen,” J. Immunol. 141:4053-4060 (1988), all of which are hereby incorporated by reference in their entirety.

As described herein, “isolated” includes separated from other components of (a) a natural source, such as a plant or animal cell or cell culture, or (b) a synthetic organic chemical reaction mixture. As used herein, “purified” means that when isolated, the isolate contains at least 95%, and in another aspect at least 98%, of a compound (e.g., a conjugate) by weight of the isolate.

An “isolated” antibody is one which has been identified and separated and/or recovered from component of its natural environment. Contaminant components of its natural environment are materials which would interfere with diagnostic or therapeutic uses for the antibody, and may include enzymes, hormones, and other proteinaceous or non-proteinaceous solutes. In some embodiments, the antibody may be purified (1) to greater than 95% by weight of antibody as determined by the Lowry method, and in some embodiments more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody may include the antibody in situ within recombinant cells since at least one component of the antibody's natural environment will not be present. Ordinarily, an isolated antibody may be prepared by at least one purification step.

An antibody which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell may be a tumor cell, e.g., a breast, ovarian, stomach, endometrial, salivary gland, lung, kidney, colon, thyroid, pancreatic or bladder cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells.

In other embodiments, the antibody is a fusion protein of an antibody, or a functionally active fragment thereof, for example in which the antibody is fused via a covalent bond (e.g., a peptide bond), at either the N-terminus or the C-terminus to an amino acid sequence of another protein (or portion thereof, preferably at least 10, 20 or 50 amino acid portion of the protein) that is not from an antibody. In one embodiment, the antibody or fragment thereof is covalently linked to the other protein at the N-terminus of the constant domain.

Antibodies include analogs and derivatives that are either modified, i.e., by the covalent attachment of any type of molecule as long as such covalent attachment permits the antibody to retain its antigen binding immunospecificity. For example, derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein. Any of numerous chemical modifications can be carried out by known techniques including, but not limited to, specific chemical cleavage, acetylation, formylation, and metabolic synthesis in the presence of tunicamycin. Additionally, the analog or derivative may contain one or more unnatural amino acids.

Antibodies may have modifications (e.g., substitutions, deletions or additions) in amino acid residues that interact with Fc receptors. In particular, antibodies may have modifications in amino acid residues identified as involved in the interaction between the anti-Fc domain and the FcRn receptor (see, e.g., International Publication No. WO 97/34631, which is incorporated herein by reference in its entirety).

In one embodiment, Ab (i.e., the antibody) is a tumor targeting antibody, an antibody fragment, a bispecific antibody or antibody fragment, a monoclonal antibody, a chimeric antibody, or a humanized antibody.

Antibodies immunospecific for a cancer cell antigen can be obtained commercially or produced by any method known to one of skill in the art such as, e.g., chemical synthesis or recombinant expression techniques. The nucleotide sequence encoding antibodies immunospecific for a cancer cell antigen can be obtained, e.g., from the GenBank database or a database like it, literature publications, or by routine cloning and sequencing.

In one embodiment, Ab (i.e., the antibody) is selected from the group consisting of anti-Her2 antibody, anti-CD20 antibody, anti-CD38 antibody, anti-IL-6 receptor antibody, anti-VEGRF2 antibody, anti-HER-2 antibody, anti-DLL3 antibody, anti-Nectin4 antibody, anti-CD33 antibody, anti-CD79b antibody, anti-CD11a antibody, anti-BCMA antibody, anti-CD22 antibody, anti-Trop2 antibody, anti-FRα antibody, anti-EpCAM antibody, anti-mesothelin antibody, anti-LIV1 antibody, oregovomab, edrecolomab, cetuximab, a humanized monoclonal antibody to the vitronectin receptor (αᵥβ₃), alemtuzumab, a humanized anti-HLA-DR antibody for the treatment of non-Hodgkin's lymphoma, 131l Lym-1, a murine anti-HLA-Dr10 antibody for the treatment of non-Hodgkin's lymphoma, a humanized anti-CD2 mAb for the treatment of Hodgkin's Disease or non-Hodgkin's lymphoma, labetuzumab, bevacizumab, ibritumomab tiuxetan, ofatumumab, panitumumab, rituximab, tositumomab, ipilimumab, gemtuzumab, humanized monoclonal antibody to the oncofecal protein receptor 5T4, M1/70 (antibody to CD11b receptor), anti-MRC1, anti GCC, anti CD32, and other antibodies.

In one embodiment, known antibodies for the treatment of cancer may be used. Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. Examples of antibodies available for the treatment of cancer include, but are not limited to, Oregovomab or OVAREX® which is a murine antibody for the treatment of ovarian cancer; Edrecolomab or panorex which is a murine IgG2a antibody for the treatment of colorectal cancer; Cetuximab (e.g., ERBITUX®) which is an anti-EGFR IgG chimeric antibody for the treatment of epidermal growth factor positive cancers, such as head and neck cancer; vitaxin, which is a humanized antibody for the treatment of sarcoma; Alemtuzumab or CAMPATH-1H, which is a humanized IgG1 antibody for the treatment of chronic lymphocytic leukemia (CLL); ONCOLYM, which is a radio labeled murine anti-HLA-Dr10 antibody for the treatment of non-Hodgkin's lymphoma; ALLOMUNE (Bio Transplant, CA) which is a humanized anti-CD2 mAb for the treatment of Hodgkin's Disease or non-Hodgkin's lymphoma; and CEA-Cide (Immunomedics, NJ) which is a humanized anti-CEA antibody for the treatment of colorectal cancer.

The terms “protein”, “polypeptide”, and “peptide” may be referred to interchangeably herein. The terms may be distinguished as follows. A protein typically refers to the end product of transcription, translation, and post-translation modifications in a cell.

A polypeptide may include a protein or a peptide. A peptide, in contrast to a protein, typically is a short polymer of amino acids, of a length typically of 100 or less amino acids.

The term “peptide” or “polypeptide” as used herein refers to proteins and fragments thereof. Peptides may include amino acid sequences. Those sequences may be written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Citrulline (Cit), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The peptides of the immunotherapy compounds may be derived from nature, or may, alternatively be designed de nova. A peptide is said to be “derivable from a naturally occurring amino acid sequence” if it can be obtained by fragmenting a naturally occurring sequence, or if it can be synthesized based upon knowledge of the sequence of the naturally occurring amino acid sequence or of the genetic material (DNA or RNA) that encodes this sequence.

The peptides of the immunotherapy compounds may or may not share substantial homology or identity with naturally occurring proteins or portions thereof (e.g., peptides). The immunotherapy compound may or may not include peptides with “substantial similarity” with naturally occurring proteins or portions thereof (e.g., peptides). A peptide with substantial similarity includes peptides with at least 70% or greater sequence homology or identity with a peptide having the same number of amino acid residues as the reference peptide.

The terms loading or “drug loading” or “payload loading” refer to the average number of payloads (“payload” and “payloads” are used interchangeably herein with “drug” and “drugs”) per antibody in an ADC molecule. Drug loading may range from 1 to 50 drugs per antibody. This is sometimes referred to as the DAR, or drug to antibody ratio. Compositions of the ADCs described herein typically have DAR's of from 1-25, and in certain embodiments, from 1-8, from 2-8, from 2-6, from 2-5 and from 2-4. Typical DAR values include 2, 4, 6, 8, and 10. The average number of drugs per antibody, or DAR value, may be characterized by conventional means such as UV/visible spectroscopy, mass spectrometry, ELISA assay, and HPLC. The quantitative DAR value may also be determined. In some instances, separation, purification, and characterization of homogeneous ADCs having a particular DAR value may be achieved by means such as reverse phase HPLC or electrophoresis. DAR may be limited by the number of attachment sites on the antibody. For example, where the attachment is a cysteine thiol, an antibody may have only one or several cysteine thiol groups, or may have only one or several sufficiently reactive thiol groups through which a linker unit may be attached. In some embodiments, the cysteine thiol is a thiol group of a cysteine residue that forms an interchain disulfide bond. In some embodiments, the cysteine thiol is a thiol group of a cysteine residue that does not form an interchain disulfide bond. Typically, fewer than the theoretical maximum of drug moieties are conjugated to an antibody during a conjugation reaction. An antibody may contain, for example, many lysine residues that do not react with a linker or linker intermediate. Only the most reactive lysine groups may react with a reactive linker reagent.

Generally, antibodies do not contain many, if any, free and reactive cysteine thiol groups which may be linked to a drug via a linker. Most cysteine thiol residues in the antibodies exist as disulfide bridges and must be reduced with a reducing agent such as dithiothreitol (DTT). The antibody may be subjected to denaturing conditions to reveal reactive nucleophilic groups such as lysine or cysteine. The loading (drug/antibody ratio) of an ADC may be controlled in several different manners, including: (i) limiting the molar excess of drug-linker relative to the antibody, (ii) limiting the conjugation reaction time or temperature, and (iii) partial or limiting reductive conditions for cysteine thiol modification. Where more than one nucleophilic group reacts with a drug-linker then the resulting product is a mixture of ADCs with a distribution of one or more drugs moieties per antibody. The average number of drugs per antibody may be calculated from the mixture by, for example, dual ELISA antibody assay, specific for antibody and specific for the drug. Individual ADCs may be identified in the mixture by mass spectroscopy, and separated by HPLC, e.g., hydrophobic interaction chromatography.

In one embodiment, the antibody may be selected from trastuzumab and a trastuzumab mutant. In some embodiments, the antibody bound via an Fc-containing or Fab-containing polypeptide engineered with an acyl donor glutamine-containing tag (e.g., Gln-containing peptide tags or Q-tags) or an endogenous glutamine made reactive (i.e., the ability to form a covalent bond as an acyl donor in the presence of an amine and a transglutaminase) by polypeptide engineering (e.g., via amino acid deletion, insertion, substitution, mutation, or any combination thereof on the polypeptide), in the presence of transglutaminase.

In certain embodiments, the present disclosure relates to any of the aforementioned antibody drug conjugates and attendant definitions, wherein the antibody drug conjugate comprises between 2, 3, 4, 5, 6, 7, 8, 9, 10, 15, 20, or 25 compounds of the present disclosure, or any number of compounds therein.

In certain embodiments, the present disclosure relates to any of the aforementioned antibody drug conjugates and attendant definitions, wherein the antibody drug conjugate comprises 3 or 4 compounds of the present disclosure.

An amino acid “derivative” includes an amino acid having substitutions or modifications by covalent attachment of a parent amino acid, such as, e.g., by alkylation, glycosylation, acetylation, phosphorylation, and the like. Further included within the contemplated meaning of “derivative” is, for example, one or more analogs of an amino acid with substituted linkages, as well as other modifications known in the art.

A “natural amino acid” refers to arginine, glutamine, phenylalanine, tyrosine, tryptophan, lysine, glycine, alanine, histidine, serine, proline, glutamic acid, aspartic acid, threonine, cysteine, methionine, leucine, asparagine, isoleucine, and valine, unless otherwise indicated by context.

A linker (sometimes referred to as “[linker]” herein) is a bifunctional compound which can be used to link a drug and an antibody to form an antibody drug conjugate (ADC). Such conjugates are useful, for example, in the formation of immunoconjugates directed against tumor associated antigens. Such conjugates may, in some embodiments, allow for the selective delivery of cytotoxic drugs to tumor cells.

A self-immolative spacer as described herein includes covalent assemblies tailored to correlate the cleavage of two chemical bonds after activation of a protective part in a precursor: Upon stimulation, the protective moiety is removed, which generates a cascade of disassembling reactions leading to the temporally sequential release of smaller molecules. See Alouane et al., “Self-Immolative Spacers: Kinetic Aspects, Structure-Property Relationships, and Applications,” Angewandte Chemie 54(26):7492-7509 (2015), which is hereby incorporated by reference in its entirety. Self-immolative spacers were created to address limitations for drug delivery, and have gained wide interest in medicinal chemistry, analytical chemistry, and material science. See Alouane et al., “Self-Immolative Spacers: Kinetic Aspects, Structure-Property Relationships, and Applications,” Angewandte Chemie 54(26:7492-7509 (2015), which is hereby incorporated by reference in its entirety.

The phrase “substantial amount” includes a majority, i.e., greater than 50% of a population, of a mixture or a sample.

The term “intracellular metabolite” refers to a compound resulting from a metabolic process or reaction inside a cell on an antibody-drug conjugate (ADC). The metabolic process or reaction may be an enzymatic process such as proteolytic cleavage of a peptide linker of the ADC. Intracellular metabolites include, but are not limited to, antibodies and free drug which have undergone intracellular cleavage after entry, diffusion, uptake, or transport into a cell.

The terms “intracellularly cleaved” and “intracellular cleavage” refer to a metabolic process or reaction inside a cell on an ADC or the like, whereby the covalent attachment, e.g., the linker, between the drug moiety and the antibody is broken, resulting in the free drug, or other metabolite of the conjugate dissociated from the antibody inside the cell. The cleaved moieties of the ADC are thus intracellular metabolites.

The term “bioavailability” refers to the systemic availability (i.e., blood/plasma levels) of a given amount of a drug administered to a patient. Bioavailability indicates measurement of both the time (rate) and total amount (extent) of drug that reaches the general circulation from an administered dosage form.

The term “cytotoxic activity” refers to a cell-killing, a cytostatic or an anti-proliferative effect of an ADC or an intracellular metabolite of said ADC. Cytotoxic activity may be expressed as the IC50 value, which is the concentration (molar or mass) per unit volume at which half the cells survive.

A “disorder” is any condition that would benefit from treatment with a drug or antibody-drug conjugate. This includes chronic and acute disorders or diseases including those pathological conditions which predispose a mammal to the disorder in question. Non-limiting examples of disorders to be treated herein include benign and malignant cancers; leukemia and lymphoid malignancies, neuronal, glial, astrocytal, hypothalamic and other glandular, macrophagal, epithelial, stromal and blastocoelic disorders; and inflammatory, angiogenic and immunologic disorders.

The terms “cancer” and “cancerous” refer to or describe the physiological condition or disorder in mammals that is typically characterized by unregulated cell growth. A “tumor” comprises one or more cancerous cells.

An infectious disease as described herein includes any viral infection, bacterial infection, fungal infection, or any combination thereof. Exemplary viral infections that may be treated in accordance with the methods described herein include, but are not limited to, coronavirus (e.g., Severe Acute Respiratory Syndrome (SARS), SARS-CoV-2 (COVID-19), Middle East Respiratory Syndrome (MERS), and the common cold), Ebola, influenza, hepatitis, Hib disease, human immunodeficiency virus (HIV), human papillomavirus (HPV), meningococcal disease, pneumococcal disease, measles, mumps, norovirus, polio, respiratory syncytial virus (RSV), rotavirus, rubella virus, shingles, West Nile virus, rabies virus, enterovirus, cytomegalovirus, herpes virus, varicella, Yellow fever, Zika virus, or any combination thereof. Exemplary bacterial infections that may be treated in accordance with the methods described herein include, but are not limited to, streptococcal disease, staphylococcal disease, diphtheria, meningococcal disease, tetanus, pertussis, pneumococcal disease, bacterial food poisoning, a sexually transmitted infection, tuberculosis, Lyme disease, botulism, or any combination thereof. Exemplary fungal infections that may be treated in accordance with the methods described herein include, but are not limited to, candidiasis, histoplasmosis, dermatophytosis, tinea pedis, aspergillosis, cryptococcal meningitis, coccidioidomycosis, or any combination thereof.

As used herein, the terms “cell”, “cell line,” and “cell culture” are used interchangeably and all such designations include progeny. The words “transformants” and “transformed cells” include the primary subject cell and cultures or progeny derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

A “patient,” as used herein, includes both humans and other animals, particularly mammals. Thus, the methods are applicable to both human therapy and veterinary applications. Examples of a “patient” include, but are not limited to, a human, rat, mouse, guinea pig, monkey, pig, goat, cow, horse, dog, cat, bird, and fowl. In some embodiments, the patient is a mammal, for example, a primate. In some embodiments, the patient is a human. In one embodiment, the patient is an infant, a juvenile, or an adult.

The terms “treat” or “treatment”, unless otherwise indicated by context, refer to therapeutic treatment and prophylactic measures to prevent relapse, wherein the object is to inhibit or slow down (lessen) an undesired physiological change or disorder, such as the development or spread of cancer or a viral infection.

For purposes of the present disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already having the condition or disorder as well as those prone to have the condition or disorder.

In the context of cancer, the term “treating” includes any or all of inhibiting growth of tumor cells, cancer cells, or of a tumor; inhibiting replication of tumor cells or cancer cells; lessening of overall tumor burden or decreasing the number of cancerous cells; and ameliorating one or more symptoms associated with the disease.

In the context of an autoimmune disease, the term “treating” includes any or all of: inhibiting replication of cells associated with an autoimmune disease state including, but not limited to, cells that produce an autoimmune antibody, lessening the autoimmune-antibody burden, and ameliorating one or more symptoms of an autoimmune disease.

In the context of an infectious disease, the term “treating” includes any or all of: inhibiting the growth, multiplication, or replication of the pathogen that causes the infectious disease and ameliorating one or more symptoms of an infectious disease.

Treatment can involve administering a compound described herein to a patient diagnosed with a disease, and may involve administering the compound to a patient who does not have active symptoms. Conversely, treatment may involve administering the compositions to a patient at risk of developing a particular disease, or to a patient reporting one or more of the physiological symptoms of a disease, even though a diagnosis of this disease may not have been made.

The terms “administer”, “administering” or “administration” in reference to a dosage form of the invention refers to the act of introducing the dosage form into the system of subject in need of treatment. When a dosage form of the invention is given in combination with one or more other active agents (in their respective dosage forms), “administration” and its variants are each understood to include concurrent and/or sequential introduction of the dosage form and the other active agents. Administration of any of the described dosage forms includes parallel administration, co-administration or sequential administration. In some situations, the therapies are administered at approximately the same time, e.g., within about a few seconds to a few hours of one another.

A “therapeutically effective” amount of the compounds described herein is typically one which is sufficient to achieve the desired effect and may vary according to the nature and severity of the disease condition, and the potency of the compound. It will be appreciated that different concentrations may be employed for prophylaxis than for treatment of an active disease. A therapeutic benefit is achieved with the amelioration of one or more of the physiological symptoms associated with the underlying disorder such that an improvement is observed in the patient, notwithstanding that the patient may still be afflicted with the underlying disorder. In the case of cancer, a therapeutically effective amount of a drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may inhibit the growth of and/or kill existing cancer cells, it may be cytostatic and/or cytotoxic. For cancer therapy, efficacy can, for example, be measured by assessing the time to disease progression (TTP) and/or determining the response rate (RR).

As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size, and sex of the subject. Optimal amounts can also be determined based on monitoring of the subject's response to treatment. The term “treatment” or “treat” may include effective inhibition, suppression or cessation of symptoms so as to prevent or delay the onset, retard the progression, or ameliorate the symptoms of a condition.

Throughout this specification the terms and substituents retain their definitions. Substituents (e.g., Rⁿ) are generally defined when introduced and retain that definition throughout the specification and in all independent claims.

C₁ to C₂₀ hydrocarbon includes alkyl, cycloalkyl, polycycloalkyl, alkenyl, alkynyl, aryl, and combinations thereof, containing from 1 to 20 carbon atoms, inclusive. Non-limiting examples include ethyl, benzyl, phenethyl, cyclohexylmethyl, camphoryl and naphthylethyl. Hydrocarbon refers to any substituent comprised of hydrogen and carbon as the only elemental constituents.

Alkyl is a subset of hydrocarbon. Unless otherwise specified, alkyl (or alkylene) is intended to include linear or branched saturated hydrocarbon structures and combinations thereof. In some embodiments, alkyl refers to alkyl groups from 1 to 20 carbon atoms, or from 1 to 10 carbon atoms, or from 1 to 8 carbon atoms, or from 1 to 6 carbon atoms, or from 1 to 5 carbon atoms, or from 1 to 4 carbon atoms. Examples of alkyl groups include methyl, ethyl, propyl, isopropyl, n-butyl, s-butyl, t-butyl and the like.

Cycloalkyl is a subset of hydrocarbon and includes cyclic hydrocarbon groups of from 3 to 8 carbon atoms. Examples of cycloalkyl groups include cy-propyl, cy-butyl, cy-pentyl, norbornyl and the like.

Oxaalkyl refers to alkyl residues in which one or more carbons (and their associated hydrogens) have been replaced by oxygen. Examples include methoxypropoxy, hydroxymethyl, hydroxyethyl, 3,6,9-trioxadecyl and the like. The term oxaalkyl is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds); it does not refer to doubly bonded oxygen, as would be found in carbonyl groups. Similarly, azaalkyl refers to alkyl residues in which one or more carbons has been replaced by nitrogen. Examples include ethylaminoethyl and methylaminopropyl, and the like.

Alkoxy or alkoxyl is a subset of oxaalkyl and refers to groups of from 1 to 20 carbon atoms attached to the parent structure through an oxygen. In some embodiments, alkyl refers to alkyl groups from 1 to 20 carbon atoms, or from 1 to 10 carbon atoms, or from 1 to 6 carbon atoms, or from 1 to 5 carbon atoms, or from 1 to 4 carbon atoms of a straight or branched configuration Examples include methoxy, ethoxy, propoxy, isopropoxy and the like. Lower-alkoxy refers to groups containing one to four carbons. For the purpose of this application, alkoxy and lower alkoxy include methylenedioxy and ethylenedioxy.

Aryl and heteroaryl mean (i) a phenyl group (or benzene) or a monocyclic 5- or 6-membered heteroaromatic ring containing 1-4 heteroatoms selected from O, N, or S; (ii) a bicyclic 9- or 10-membered aromatic or heteroaromatic ring system containing 0-4 heteroatoms selected from O, N, or S; or (iii) a tricyclic 13- or 14-membered aromatic or heteroaromatic ring system containing 0-5 heteroatoms selected from O, N, or S. The aromatic 6- to 14-membered carbocyclic rings include, e.g., benzene, naphthalene, indane, tetralin, and fluorene and the 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole. As used herein aryl and heteroaryl refer to residues in which one or more rings are aromatic, but not all need be. In some embodiments, aryl refers to a phenyl group. In some embodiments, heteroaryl refers to pyridine, imidazole, pyrimidine, indole, thiophene, benzopyranone, thiazole, furan, benzimidazole, quinoline, isoquinoline, quinoxaline, pyrimidine, pyrazine, tetrazole and pyrazole. In other embodiments, heteroaryl refers to pyridine, pyridazine, pyrazine, or pyrimidine. In still other embodiments, heteroaryl refers to pyridine.

Heterocycle means a cycloalkyl or aryl carbocycle residue in which from one to four carbons is replaced by a heteroatom selected from the group consisting of N, O and S. The nitrogen and sulfur heteroatoms may optionally be oxidized, and the nitrogen heteroatom may optionally be quaternized. Unless otherwise specified, a heterocycle may be non-aromatic or aromatic. Non-limiting examples of heterocycles that fall within the scope of the invention include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. It is to be noted that heteroaryl is a subset of heterocycle in which the heterocycle is aromatic. Examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

A nitrogen heterocycle is a heterocycle containing at least one nitrogen in the ring; it may contain additional nitrogens, as well as other heteroatoms. Non-limiting examples include piperidine, piperazine, morpholine, pyrrolidine and thiomorpholine. Nitrogen heteroaryl is a subset of nitrogen heterocycle; examples include pyridine, pyrrole and thiazole.

The term “halogen” means fluorine, chlorine, bromine or iodine atoms. In one embodiment, halogen may be a fluorine or chlorine atom.

Unless otherwise specified, acyl refers to formyl and to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. Examples include acetyl, benzoyl, propionyl, isobutyryl and the like. Lower-acyl refers to groups containing one to four carbons. The double bonded oxygen, when referred to as a substituent itself is called “oxo”.

As used herein, the term “optionally substituted” may be used interchangeably with “unsubstituted or substituted.” The term “substituted” refers to the replacement of one or more hydrogen atoms in a specified group with a specified radical. For example, “substituted aryl” or “substituted heteroaryl” refers to aryl or heteroaryl wherein one or more H atoms in each residue are replaced with halogen, haloalkyl, alkyl, alkoxy, or haloalkoxy.

The compounds described herein may contain asymmetric centers and may thus give rise to enantiomers, diastereomers, and other stereoisomeric forms which may be defined in terms of absolute stereochemistry as (R)- or (S)-. The present invention is meant to include all such possible diastereomers as well as their racemic and optically pure forms. Optically active (R)- and (S)-isomers may be prepared using homo-chiral synthons or homo-chiral reagents, or optically resolved using conventional techniques. When the compounds described herein contain olefinic double bonds or other centers of geometric asymmetry, and unless specified otherwise, it is intended to include both (E)- and (Z)-geometric isomers. Likewise, all tautomeric forms are intended to be included.

The graphic representations of racemic, ambiscalemic and scalemic or enantiomerically pure compounds used herein are a modified version of the denotations taken from Maehr J. Chem. Ed. 62, 114-120 (1985): simple lines provide no information about stereochemistry and convey only connectivity; solid and broken wedges are used to denote the absolute configuration of a chiral element; solid and broken bold lines are geometric descriptors indicating the relative configuration shown but not necessarily denoting racemic character; and wedge outlines and dotted or broken lines denote enantiomerically pure compounds of indeterminate absolute configuration. For example, the graphic representation

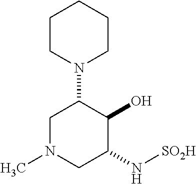

indicates either, or both, of the two trans:trans enantiomers:

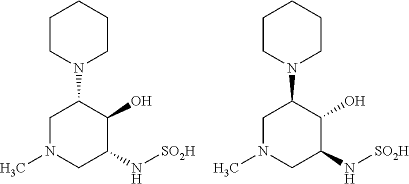

in any ratio, from pure enantiomers to racemates. The graphic representation:

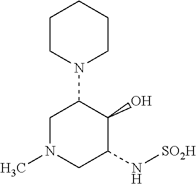

indicates a single enantiomer of unknown absolute stereochemistry, i.e., it could be either of the two preceding structures, as a substantially pure single enantiomer. And, finally, the representation:

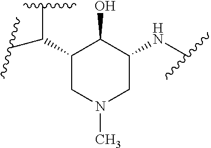 [SMILES: *N[C@@H]1CN(C)C[C@H](C(*)*)[C@H]1O]

indicates a pure (R,R,S) absolute configuration. For the purpose of the present disclosure, a “pure” or “substantially pure” enantiomer is intended to mean that the enantiomer is at least 95% of the configuration shown and 5% or less of other enantiomers. Similarly, a “pure” or “substantially pure” diastereomer is intended to mean that the diastereomer is at least 95% of the relative configuration shown and 5% or less of other diastereomers. In some embodiments, the purity of the compound is at least 99%.

In any of these possibilities, compounds can be a single stereoisomer or a mixture. If a mixture, the mixture will most commonly be racemic, but it need not be. Substantially pure single stereoisomers of biologically active compounds such as those described herein often exhibit advantages over their racemic mixture.

Enantiomerically pure means greater than 80 e.e., and preferably greater than 90 e.e. For the purpose of the present disclosure, a “pure” or “substantially pure” stereoisomer is intended to mean that the stereoisomer is at least 95% of the configuration shown and 5% or less of other stereoisomers, or at least 97% of the configuration shown and 3% or less of other stereoisomers, or at least 99% of the configuration shown and 1% or less of other stereoisomers.

It may be found upon examination that certain species and genera are not patentable to the inventors in this application. In this case, the exclusion of species and genera in applicants' claims are to be considered artifacts of patent prosecution and not reflective of the inventors' concept or description of their invention, which encompasses all members of the genus I that are not in the public's possession.

As used herein, and as would be understood by the person of skill in the art, the recitation of “a compound”—unless expressly further limited—is intended to include salts of that compound. In a particular embodiment, the term “compound of formula” refers to the compound or a pharmaceutically acceptable salt thereof.

The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable pharmaceutically acceptable acid addition salts for the compounds of the present invention include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

Also provided herein is a pharmaceutical composition comprising a compound disclosed above, or a pharmaceutically acceptable salt form thereof, and a pharmaceutically acceptable carrier, diluent, or excipient.

While it may be possible for the compounds disclosed herein to be administered as the raw chemical, it is preferable to present them as a pharmaceutical composition. According to a further aspect, the present invention provides a pharmaceutical composition comprising a compound of formula I or a pharmaceutically acceptable salt thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof. In one embodiment, the pharmaceutically acceptable carrier is selected from the group consisting of a liquid filler, a solid filler, a diluent, an excipient, a solvent, and an encapsulating material.

Pharmaceutically acceptable carriers (e.g., additives such as diluents, immunostimulants, adjuvants, antioxidants, preservatives and solubilizing agents) are nontoxic to the cell or subject being exposed thereto at the dosages and concentrations employed. Examples of pharmaceutically acceptable carriers include water, e.g., buffered with phosphate, citrate and another organic acid. Representative examples of pharmaceutically acceptable excipients that may be useful in the present disclosure include antioxidants such as ascorbic acid; low molecular weight (less than about 10 residues) polypeptides; proteins, such as serum albumin, gelatin, or immunoglobulins; adjuvants (selected so as to avoid adjuvant-induced toxicity, such as a (3-glucan as described in U.S. Pat. No. 6,355,625, which is hereby incorporated by reference in its entirety, or a granulocyte colony stimulating factor (GCSF)); hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants such as TWEEN®, polyethylene glycol (PEG), and PLURONICS®.

In one embodiment, the composition may further comprise an adjuvant. Suitable adjuvants are known in the art and include, without limitation, flagellin, Freund's complete or incomplete adjuvant, aluminum hydroxide, lysolecithin, pluronic polyols, polyanions, peptides, oil emulsion, dinitrophenol, iscomatrix, and liposome polycation DNA particles.

The formulations include those suitable for parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound disclosed herein or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use.

Extemporaneous injection solutions and suspensions may be prepared from sterile powders, granules and tablets of the kind previously described.

The term “package insert” is used to refer to instructions customarily included in commercial packages of therapeutic products, that contain information about the indication(s), usage, dosage, administration, contraindications, and/or warnings concerning the use of such therapeutic products.

It will be recognized that the compounds of this invention can exist in radiolabeled form, i.e., the compounds may contain one or more atoms containing an atomic mass or mass number different from the atomic mass or mass number usually found in nature.

Radioisotopes of hydrogen, carbon, phosphorous, fluorine, and chlorine include ²H, ³H, ¹³C, ⁴C, ¹⁵N ³⁵S, ¹⁸F, and ³⁶Cl, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Compounds that contain isotopes ¹¹C, ¹³N, ¹⁵O and ¹⁸F are well suited for positron emission tomography. Radiolabeled compounds of formula I of this invention and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

Preparation of compounds can involve the protection and deprotection of various chemical groups. The need for protection and deprotection, and the selection of appropriate protecting groups, can be readily determined by one skilled in the art. Suitable groups for that purpose are discussed in standard textbooks in the field of chemistry, such as Protective Groups in Organic Synthesis by T. W. Greene and P. G. M. Wuts [John Wiley & Sons, New York, 1999], in Protecting Group Chemistry, 1^{st} Ed., Oxford University Press, 2000; and in March's Advanced Organic chemistry: Reactions, Mechanisms, and Structure, 5^{th} Ed., Wiley-Interscience Publication, 2001.

### EXAMPLES

### Example 1: Preparation of 1-(4-(aminomethyl)benzyl)-2-butyl-1H-imidazo[4,5-c]quinolin-4-amine (E66)

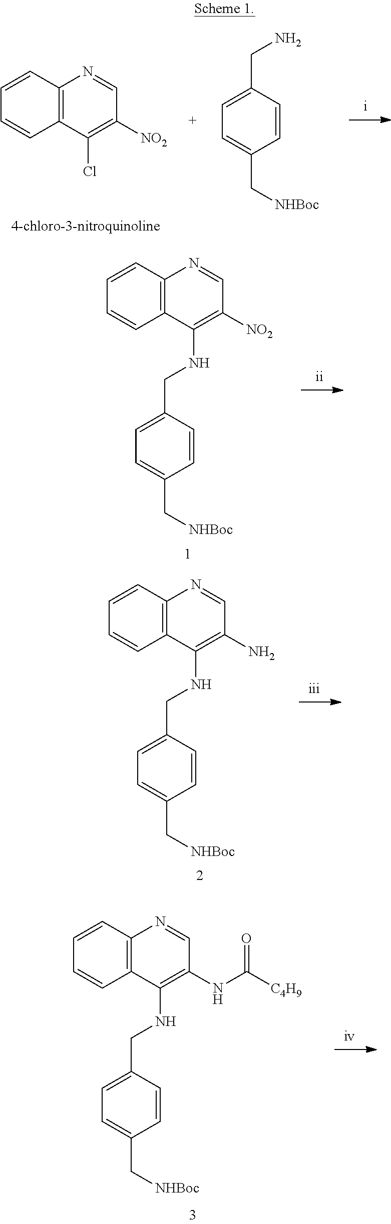

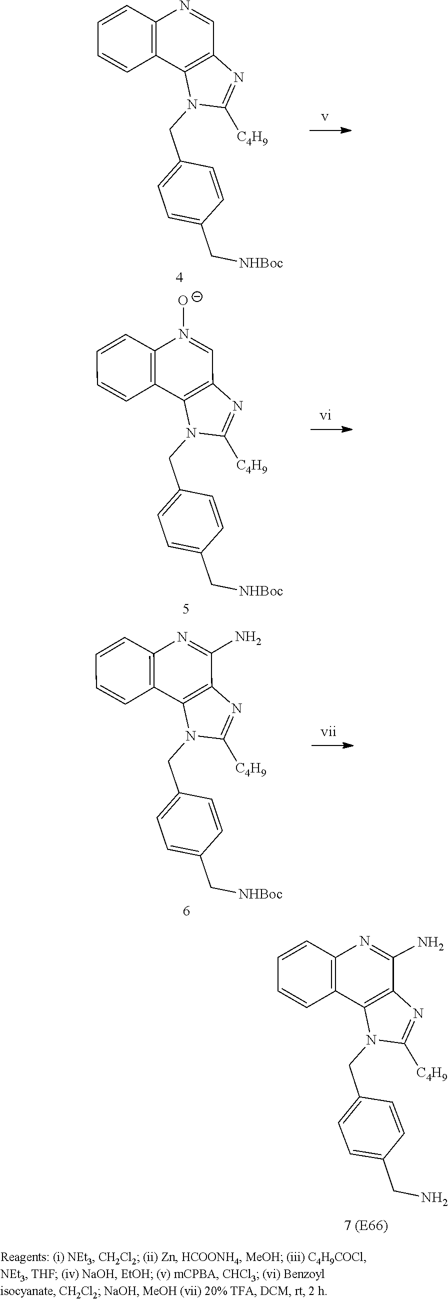

Step 1. tert-butyl (4-(((3-nitroquinolin-4-yl)amino)methyl)benzyl)carbamate: Triethylamine (72.8 mg, 100 μL, 1.5 Eq, 719 μmol) and tert-butyl (4-(aminomethyl)benzyl) carbamate (136 mg, 1.2 eq, 575 μmol) were added to a solution of 4-chloro-3-nitroquinoline (100 mg, 1 eq, 479 μmol) in dichloromethane (2 mL). The reaction mixture was refluxed at 40° C. for 45 minutes. The reaction progress was monitored by UPLC. The product (@ 2.83 min) started forming immediately. Complete conversion of the reactants to the desired product was achieved by 45 min. Water was added to the residue and the product was extracted with CH₂Cl₂ (3×). The extracts were washed with water, dried over MgSO₄, and evaporated in vacuo to obtain the desired compound (195.8 mg, ˜100%) as a bright yellow solid. [Alternatively, the yellow precipitate formed after the reaction is filtered, washed with water and dried under vacuum to obtain the target compound.]

Step 2. tert-butyl (4-(((3-aminoquinolin-4-yl)amino)methyl)benzyl)carbamate: Without further purification, a suspension of the material from step 1 (tert-butyl (4-(((3-nitroquinolin-4-yl)amino)methyl)benzyl)carbamate, 100 mg, 1 Eq, 245 μmol) in MeOH (2.2 mL) was treated with zinc dust (80.0 mg, 5 Eq, 1.22 mmol) and ammonium formate (77.2 mg, 5 Eq, 1.22 mmol). The reaction mixture was stirred at room temperature for 20 min to give a grey solution. Reaction progression was monitored by UPLC. Product began forming immediately. Upon completion, the reaction mixture was filtered through celite and the solvent was evaporated in vacuo. The residue was dissolved in water, extracted with EtOAc (3×20 mL), washed with water and dried over MgSO4. The solvent was removed under vacuum to obtain 82.2 mg (88.7%) of the desired compound as a stick puffy bright yellowish substance.

Step 3. tert-butyl (4-(((3-pentanamidoquinolin-4-yl)amino)methyl)benzyl)carbamate: To the crude product of step 2 (tert-butyl (4-(((3-aminoquinolin-4-yl)amino)methyl) benzyl)carbamate, 41.1 mg, 1 Eq, 109 μmol) in anhydrous EtOAc (40 mL) were added triethylamine (14.3 mg, 19.7 μL, 1.3 Eq, 141 μmol) and Valeryl chloride (14.4 mg, 14.2 μL, 1.1 Eq, 119 μmol). The reaction mixture was refluxed for 30 min. The reaction was monitored by UPLC. The desired product precipitated from solution by 30 minutes. The solvent was evaporated under reduced pressure and the residue was dissolved in EtOAc and washed with water. The organic fraction was dried over MgSO₄ and evaporated in vacuo to obtain the intermediate amide as a brown oil.

Step 4. tert-butyl (4-((2-butyl-1H-imidazo[4,5-c]quinolin-1-yl)methyl)benzyl)carbamate: Without further purification, the material from step 3 (tert-butyl (4-(((3-pentanamidoquinolin-4-yl)amino)methyl)benzyl)carbamate, 222 mg, 1 Eq, 479 μmol) was dissolved in EtOH (20 mL) and sodium hydroxide (38.3 mg, 2 Eq, 958 μmol) in water (200 μL) was added. The reaction mixture was refluxed at for 4-6 h and monitored by UPLC. Upon completion of the reaction, the solvent was removed under reduced pressure, the residue was dissolved in EtOAc and washed with water. The organic layer was dried over MgSO4, evaporated to dryness. The crude extract was purified using column chromatography (Hex-EtOAc) to obtain the desired compound (23.7 mg, 11.1%) as an amber colored oily substance.

Step 5. 1-(4-(((tert-butoxycarbonyl)amino)methyl)benzyl)-2-butyl-1H-imidazo[4,5-c]quinoline 5-oxide To the product of step 4 (tert-butyl (4-((2-butyl-1H-imidazo[4,5-c]quinolin-1-yl)methyl)benzyl)carbamate, 31.5 mg, 1 Eq, 70.9 μmol) in DCM/MeOH (19:1, 3.5 mL) was added 3-chlorobenzoperoxoic acid (116 mg, 9.5 Eq, 673 μmol) and the reaction was stirred at 50° C. for 3 h. The solvent was removed and the crude residue was redissolved in DMA and purified by HPLC to give 12.3 mg (37.7%) an amber oily substance.

Step 6. tert-butyl (4-((4-amino-2-butyl-1H-imidazo[4,5-c]quinolin-1-yl)methyl)benzyl)carbamate: To a solution of the product of step 5 (12.3 mg, 1 Eq, 26.7 μmol) in CH2Cl2 (400 μL) was added benzoyl isocyanate {1.3 eq} and stirred at 45° C. for 3 h. (b) The solvent was evaporated in vacuo and the residue was dissolved in anhydrous MeOH (50 μL), followed by the addition of excess sodium methoxide (350 μL), and further stirred at 80° C. for 2 h. The solvent was removed under reduced pressure, partitioned between CH2Cl2 and water, the organic layer dried over MgSO4 and concentrated. The residue was purified using HPLC to give 10.7 mg (87%) of the title compound as a white solid.

Step 7. 1-(4-(aminomethyl)benzyl)-2-butyl-1H-imidazo[4,5-c]quinolin-4-amine: To a solution of the product of step 6 (10 mg, 1 Eq, 22 μmol) in DCM (273 μL) was added 20% v/v TFA (1 mL) and stirred at rt for 2 h. The reaction was monitored by UPLC with product peak eluting at 2.60 min. The reaction mixture was evaporated in-vacuo, the crude extract was redissolved in DMA and purified using HPLC, yielding the title compound as a white solid (4.9 mg, 62.8%) LC-MS: m/z 359.48 [M+1]⁺; Retention time=1.99 min. ¹H NMR (400 MHz, DMSO) δ_{H}/ppm 11.57 (s, 1H), 8.14 (dd, J=5.5, 2.6 Hz, 1H), 7.43 (s, 2H), 7.41 (d, J=1.9 Hz, 2H), 7.39 (s, 1H), 7.37-7.31 (m, 2H), 7.10 (dd, J=18.7, 8.3 Hz, 2H), 5.85 (s, 1H), 3.99 (dt, J=11.2, 5.7 Hz, 2H), 3.43 (s, 1H), 1.72 (dt, J=15.3, 8.2 Hz, 2H), 1.41-1.34 (m, 2H), 0.90-0.85 (m, 3H).

### Example 2. Preparation of 1-(4-aminobenzyl)-2-butyl-1H-imidazo[4,5-c]quinolin-4-amine (E104)

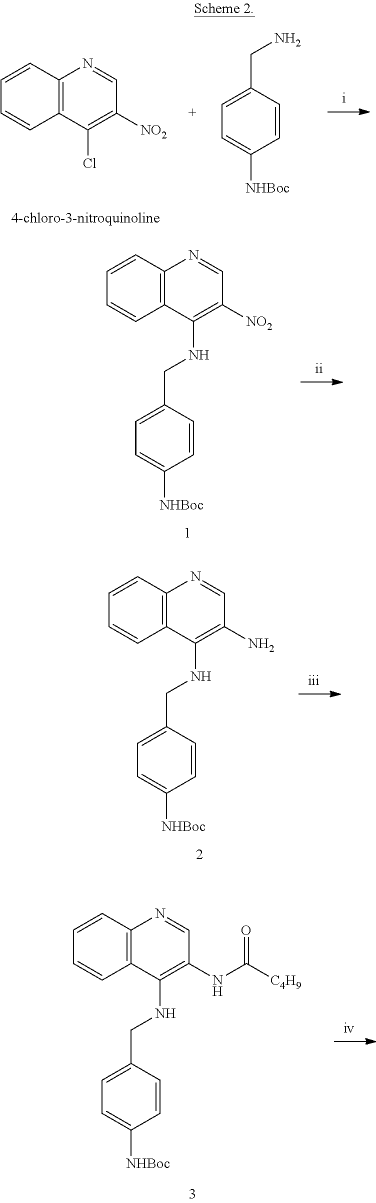

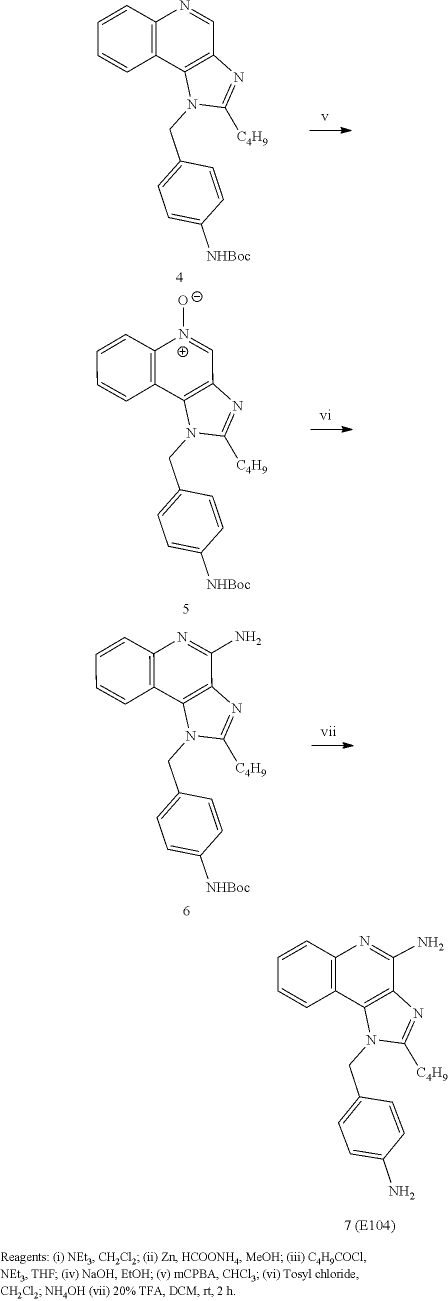

Step 1. tert-butyl (4-(((3-nitroquinolin-4-yl)amino)methyl)phenyl)carbamate: To a solution of 4-chloro-3-nitroquinoline (1.55 g, 1 Eq, 7.43 mmol) in DCM (15 mL) was added tert-butyl (4-(aminomethyl)phenyl)carbamate (1.65 g, 1 Eq, 7.43 mmol) and triethylamine (1.13 g, 1.55 mL, 1.5 Eq, 11.1 mmol). The mixture was refluxed at 40° C. for 1 h. The reaction progress was monitored by UPLC. Complete conversion of the reactants to the desired product was achieved by 45 min, forming a yellowish precipitate. The reaction mixture was cooled to rt, filtered and dried under vacuum to obtain the desired compound (3.35 g) as a bright yellow solid. HPLC rt=2.86 min; m/z=395.3 [M+H].

Step 2. tert-butyl (4-(((3-aminoquinolin-4-yl)amino)methyl)phenyl)carbamate: To a suspension of the product of step 1 (tert-butyl (4-(((3-nitroquinolin-4-yl)amino)methyl)phenyl)carbamate, 3.35 g, 1 Eq, 8.49 mmol) in MeOH (2.2 mL) were added zinc (2.78 g, 5 Eq, 42.5 mmol) and Ammonium formate (2.68 g, 5 Eq, 42.5 mmol). The reaction mixture was stirred at room temperature for 20 min (to give a grey suspension) and monitored by UPLC. Product began forming immediately. After 20 minutes, the reaction mixture was filtered through celite and the solvent was evaporated in vacuo. The residue was dissolved in water, extracted with EtOAc (3×20 mL), washed with water and dried over MgSO4. The solvent was removed under vacuum to obtain 3.1 g (100%) of the title compound as a sticky, puffy, deep yellowish substance. HPLC rt=2.14; m/z=365.3 [M+H].

Step 3. tert-butyl (4-(((3-pentanamidoquinolin-4-yl)amino)methyl)phenyl)carbamate: To the crude product of step 2 (tert-butyl (4-(((3-aminoquinolin-4-yl)amino)methyl)phenyl)carbamate, 1500 mg, 1 Eq, 4.116 mmol) in anhydrous EtOAc (40 mL), cooled to 0° C., was added previously cooled triethylamine (541.4 mg, 746 μL, 1.3 Eq, 5.351 mmol). The reaction was stirred at rt for 15 mins. Thereafter, Valeryl chloride (545.9 mg, 537.3 μL, 1.1 Eq, 4.527 mmol) in EtOAc (20 mL) was added dropwise at −10-0° C. and the reaction mixture was further stirred for 30 min, and monitored by UPLC. The reaction mixture was washed with water, organic fraction dried over MgSO4 and evaporated under reduced pressure to obtain 1.85 g of the title as a brown oil that became a fluffy brown solid after drying under vacuum. LCMS rt=2.30 min; m/z=449.4 [M+H].

Step 4. tert-butyl (4-((2-butyl-1H-imidazo[4,5-c]quinolin-1-yl)methyl)phenyl)carbamate: The crude product of step 3 (tert-butyl (4-(((3-pentanamidoquinolin-4-yl)amino)methyl)phenyl)carbamate, 1.846 g, 1 Eq, 4.115 mmol) was dissolved in EtOH (26 mL) and treated with sodium hydroxide (329.2 mg, 2 Eq, 8.231 mmol) in H2O (4 mL). The reaction mixture was refluxed at 80° C. for 5 h and progress was monitored by UPLC. Upon completion, the solvent was removed under reduced pressure and the residue was dissolved in EtOAc and washed with water. The organic layer was dried over MgSO₄ and evaporated to dryness. A solution of saturated NaHCO₃ was added and the product was extracted with EtOAc, dried over MgSO₄ and dried in vacuo. The dried brown oily crude extract (1700 mg) was used for the next step without further purification. LCMS rt=2.63; m/z=431.3 [M+H].

Step 5. 1-(4-((tert-butoxycarbonyl)amino)benzyl)-2-butyl-1H-imidazo[4,5-c]quinoline 5-oxide: Without further purification, tert-butyl (4-((2-butyl-1H-imidazo[4,5-c]quinolin-1-yl)methyl)phenyl)carbamate (1700 mg, 1 Eq, 3.948 mmol)} in CH2Cl2/MeOH (19:1, 14 mL) was treated with 3-chlorobenzoperoxoic acid (2.725 g, 4 Eq, 15.79 mmol) and stirred at 50° C. for 3 h. The reaction mixture was evaporated under reduced pressure, extracted with EtOAc, wash successively with saturated solution of NaHCO₃ and water. The organic fraction dried over MgSO₄ and the solvent evaporated at reduced pressure to obtain the crude product Yield (1.8 g, 100%). 20 mg of the crude product was dissolved in DMA (500 μL) and purified on HPLC for characterization. The remaining material was used for the next step without further purification. LCMS rt=3.08; m/z=447.3 [M+H].

Step 6. tert-butyl (4-((4-amino-2-butyl-1H-imidazo[4,5-c]quinolin-1-yl)methyl)phenyl)carbamate: To a solution of crude tert-butyl (4-((2-butyl-5-(l1-oxidaneyl)-1H-514-imidazo[4,5-c]quinolin-1-yl)methyl)phenyl)carbamate (1742 mg, 1 Eq, 3.901 mmol) in CH2Cl2 (125 mL) at 0-10° C. was added 4-methylbenzenesulfonyl chloride (966.8 mg, 1.3 Eq, 5.071 mmol) dropwise followed by the addition of 28-38% Ammonium hydroxide (125.8 g, 0.14 L, 920 Eq, 3.589 mol). The mixture was stirred at room temperature for 2 h and monitored by UPLC. Upon completion, the reaction was diluted with water and the organic fraction was separated and washed with 2M HCl, dried over MgSO₄ and solvent removed in vacuo to obtain the 506 mg of crude product which was used for the next step without further purification. LCMS rt=2.66 min; m/z=446.3 [M+H].

Step 7. 1-(4-aminobenzyl)-2-butyl-1H-imidazo[4,5-c]quinolin-4-amine: To a solution of crude extract of tert-butyl (4-((4-amino-2-butyl-1H-imidazo[4,5-c]quinolin-1-yl)methyl)phenyl)carbamate (506 mg, 1 Eq, 49 μmol) in CH2Cl2 (9 mL) was added TFA (2.3 mL). The mixture was stirred at rt for 1 h and the solvent was removed in vacuo. A portion of the residue was purified by HPLC to give the title compound, while the remaining crude residue was used directly in subsequent steps. LC-MS: m/z 345.4 [M+1]⁺; Retention time=2.39 min. ¹H NMR (400 MHz, DMSO) δ_{H}/ppm 7.98 (dd, J=8.3, 1.3 Hz, 2H), 7.79 (dd, J=8.4, 1.3 Hz, 2H), 7.39 (d, J=1.3 Hz, 2H), 7.36 (s, 2H), 6.94 (dd, J=26.7, 8.6 Hz, 2H), 5.87 (s, 1H), 2.99-2.94 (m, 2H), 2.86 (d, J=63.6 Hz, 2H), 2.02 (d, J=51.5 Hz, 2H), 0.87 (t, J=7.3 Hz, 3H).

### Example 3. Preparation of (4-amino-1-(4-aminobenzyl)-1H-imidazo[4,5-c]quinolin-2-yl)methanol [E136]

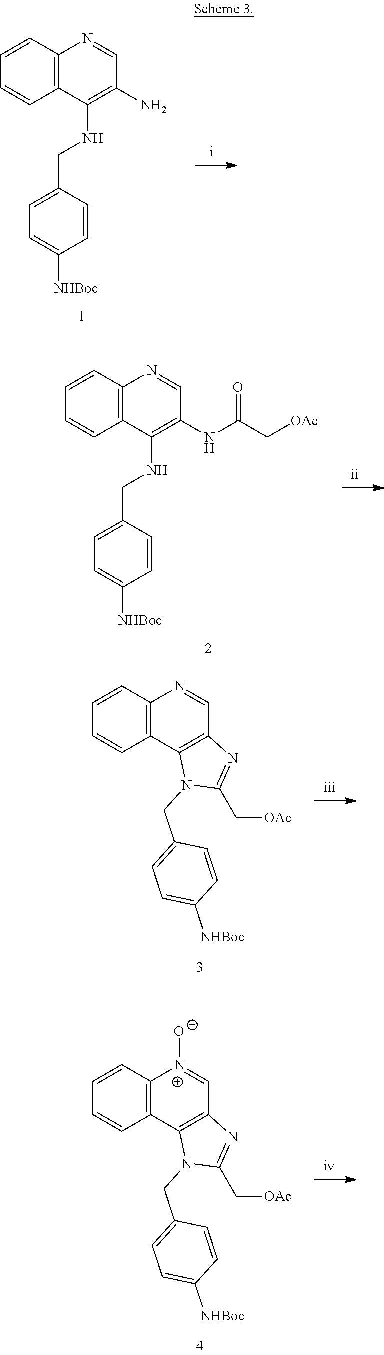 [SMILES: CC(C)(C)OC(=O)Nc1ccc(CNc2c(N)cnc3ccccc23)cc1 ; CC(=O)OCC(=O)Nc1cnc2ccccc2c1NCc1ccc(NC(=O)OC(C)(C)C)cc1 ; CC(=O)OCc1nc2cnc3ccccc3c2n1Cc1ccc(NC(=O)OC(C)(C)C)cc1 ; CC(=O)OCc1nc2c[n+]([O-])c3ccccc3c2n1Cc1ccc(NC(=O)OC(C)(C)C)cc1]

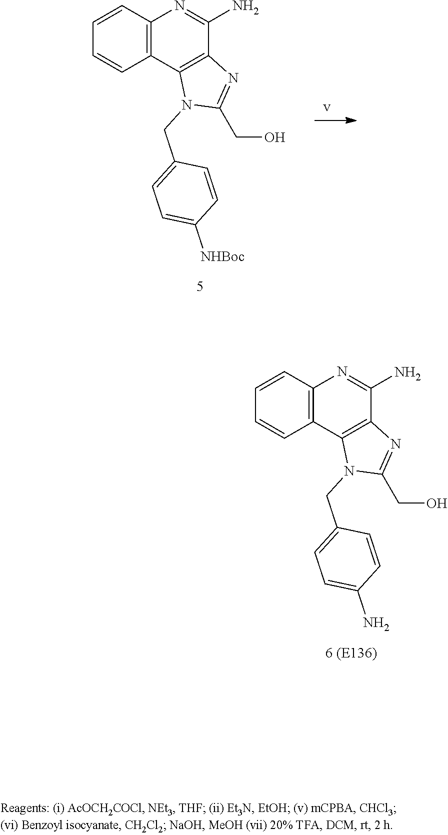 [SMILES: CC(C)(C)OC(=O)Nc1ccc(Cn2c(CO)nc3c(N)nc4ccccc4c32)cc1 ; Nc1ccc(Cn2c(CO)nc3c(N)nc4ccccc4c32)cc1]

Step 1. 2-((4-((4-((tert-butoxycarbonyl)amino)benzyl)amino)quinolin-3-yl)amino)-2-oxoethyl acetate: To tert-butyl (4-(((3-aminoquinolin-4-yl)amino)methyl)phenyl)carbamate (Example 2, step 2) (2.0 g, 1 Eq, 5.28 mmol) in EtOAc (15 mL) cooled to −10-0° C. was added previously cooled TEA triethylamine (2.21 mL, 3 Eq, 15.9 mmol). Thereafter, 2-chloro-2-oxoethyl acetate (938 mg, 1.5 Eq) in EtOAc (7.5 mL) was added dropwise at −10-0° C. and the reaction mixture was further stirred for 30 min. The reaction was monitored by UPLC. The reaction mixture was washed with water and the organic fraction was dried over MgSO₄ and evaporated in vacuo to obtain the title compound as a brown oil that became a fluffy brown solid that was used directly in the next step. HPLC rt=1.93 mg; m/z=479.3 [M+H]. [Note that the produce was contaminated with some cyclized product due to the basic reaction conditions.]

Step 2. (1-(4-((tert-butoxycarbonyl)amino)benzyl)-1H-imidazo[4,5-c]quinolin-2-yl)methyl acetate: The crude product of the previous step (˜5.3 mmol) was dissolved in EtOH (15 mL) and treated with TEA (4.956 g, 6.83 mL, 7 Eq, 48.98 mmol). The mixture was heated to 80° C. for 2 h. The reaction was monitored with UPLC until complete conversion. The solvent was evaporated under reduced pressure to give 1500 mg of crude product. HPLC rt=2.96 min; m/z=447.3 [M+H].

Step 3. 2-(acetoxymethyl)-1-(4-((tert-butoxycarbonyl)amino)benzyl)-1H-imidazo[4,5-c]quinoline 5-oxide: Without further purification, 2-((4-((4-((tert-butoxycarbonyl)amino)benzyl)amino)quinolin-3-yl)amino)-2-oxoethyl acetate (1500.00 mg, 1 Eq, 3.2291 mmol) in CH2Cl2/MeOH (19:1, 15 mL) was treated with 3-chlorobenzoperoxoic acid (1.6717 g, 3 Eq, 9.6874 mmol) and stirred at 50° C. for 3 h. The reaction mixture was evaporated under reduced pressure, extracted with EtOAc, washed with water. The organic fraction dried over MgSO4 and the solvent evaporated at reduced pressure to obtain 500 mg of crude product. HPLC rt=3.14 min; m/z=463.2 [M+H].

Step 6. tert-butyl (4-((4-amino-2-(hydroxymethyl)-1H-imidazo[4,5-c]quinolin-1-yl)methyl)phenyl)carbamate: A solution of crude material from the above reaction (500.00 mg, 1 Eq, 1.0811 mmol) in CH₂Cl₂ (13 mL) was treated with benzoyl isocyanate (209.61 mg, 1.3 Eq, 1.4054 mmol) and stirred at 45° C. for 3 h. The solvent was evaporated in vacuo and the residue was dissolved in anhydrous MeOH (5 mL), followed by the addition of excess sodium methoxide (3 mL). After stirring at 80° C. for 2 h, the solvent was removed under reduced pressure, the residue was partitioned between CH₂Cl₂ and water, and the organic layer dried over MgSO₄ and concentrated. The residue was used for the next step without further purification. HPLC rt=2.64; m/z=420.4.

Step 7. (4-amino-1-(4-aminobenzyl)-1H-imidazo[4,5-c]quinolin-2-yl)methanol: To a solution of crude tert-butyl (4-((4-amino-2-(hydroxymethyl)-1H-imidazo[4,5-c]quinolin-1-yl)methyl)benzyl)carbamate (50 mg, 1 Eq) in a round-bottomed flask with stir bar was added TFA (1 mL) and the reaction was stirred at ambient temperature for 1 h. The solvent was evaporated under reduced pressure to afford a crude residue that was purified by HPLC to give the desired product as a yellow oil. HPLC rt=1.04; m/z=320.2 [M+H].

### Example 4. Preparation of (4-amino-1-(4-(aminomethyl)benzyl)-1H-imidazo[4,5-c]quinolin-2-yl)methanol (E75)

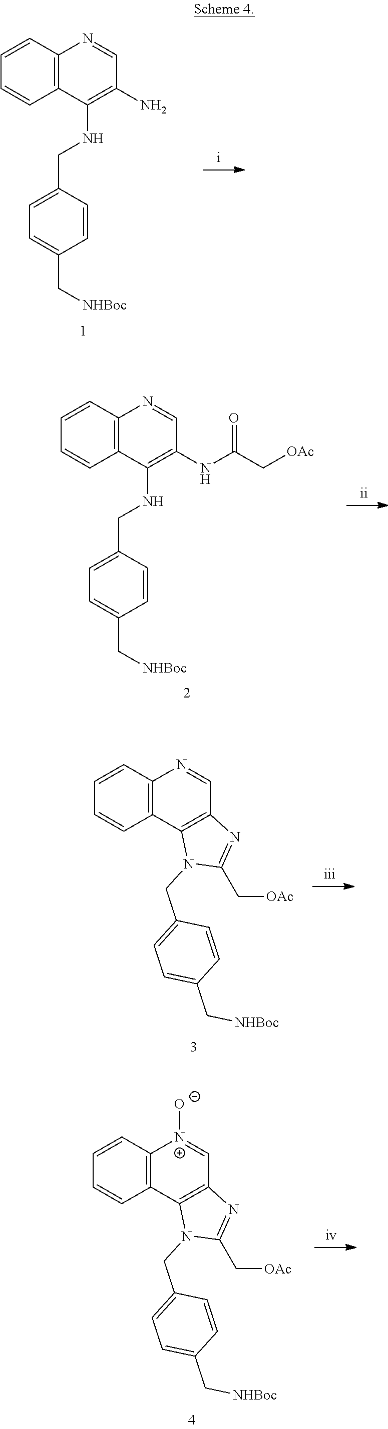 [SMILES: CC(C)(C)OC(=O)NCc1ccc(CNc2c(N)cnc3ccccc23)cc1 ; CC(=O)OCC(=O)Nc1cnc2ccccc2c1NCc1ccc(CNC(=O)OC(C)(C)C)cc1 ; CC(=O)OCc1nc2cnc3ccccc3c2n1Cc1ccc(CNC(=O)OC(C)(C)C)cc1 ; CC(=O)OCc1nc2c[n+]([O-])c3ccccc3c2n1Cc1ccc(CNC(=O)OC(C)(C)C)cc1]

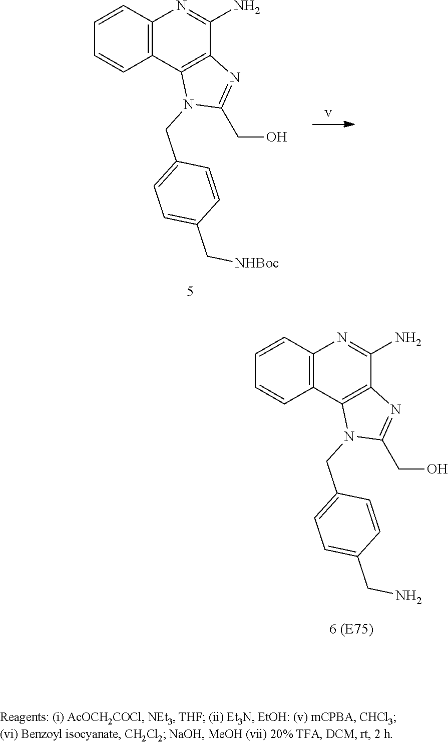 [SMILES: CC(C)(C)OC(=O)NCc1ccc(Cn2c(CO)nc3c(N)nc4ccccc4c32)cc1 ; NCc1ccc(Cn2c(CO)nc3c(N)nc4ccccc4c32)cc1]

Step 1. 2-((4-((4-(((tert-butoxycarbonyl)amino)methyl)benzyl)amino)quinolin-3-yl)amino)-2-oxoethyl acetate: Tert-butyl (4-(((3-aminoquinolin-4-yl)amino)methyl)benzyl)carbamate (Example 1, step 2) (2.00 g, 1 Eq, 5.28 mmol) was dissolved in EtOAc (15 mL) and cooled to −10-0° C. Previously cooled triethylamine (1.60 g, 2.21 mL, 3 Eq, 15.9 mmol) was added and the reaction was stirred for 15 mins at which time 2-chloro-2-oxoethyl acetate (938 mg, 739 μL, 1.3 Eq, 6.87 mmol) in EtOAc (7.5 mL) was added dropwise, at −10-0° C. The reaction mixture was stirred for 30 min and monitored by UPLC. The organic solution was washed with water and the organic fraction was dried over MgSO4 and evaporated in vacuo to obtain the title product (2.45 g) as a brown oil that became a fluffy brown solid after drying under vacuum. HPLC rt=1.93; m/z=479.3 [M+H].

Step 2. 2-((4-((4-(((tert-butoxycarbonyl)amino)methyl)benzyl)amino)quinolin-3-yl)amino)-2-oxoethyl acetate: The crude product from the previous step (2.450 g, 1 Eq, 5.120 mmol) was dissolved in EtOH (15 mL) and treated with triethylamine (3.626 g, 5.00 mL, 7 Eq, 35.84 mmol). The reaction was heated to 80° C. for 12 h and monitored with UPLC. The reaction was partitioned between 1M HCl and EtOAc. The organic fraction was dried over MgSO4 and evaporated under reduced pressure to afford the crude product which was used in subsequent steps without purification. HPLC rt=2.42; m/z=461.3.

Step 3. 2-(acetoxymethyl)-1-(4-(((tert-butoxycarbonyl)amino)methyl)benzyl)-1H-imidazo[4,5-c]quinoline 5-oxide: The product of the previous step (1000.00 mg, 1 Eq, 2.1714 mmol) in CH2Cl2/MeOH (19:1, 12 mL) was treated with 3-chlorobenzoperoxoic acid (1.1241 g, 3 Eq, 6.5142 mmol) and stirred at 50° C. for 3 h. The reaction mixture was evaporated under reduced pressure, extracted with EtOAc, washed with water. The organic fraction dried over MgSO4 and the solvent evaporated at reduced pressure to obtain the crude product which was used in the next step without further purification. HPLC rt=3.06; m/z=477.3.

Step 4. tert-butyl (4-((4-amino-2-(hydroxymethyl)-1H-imidazo[4,5-c]quinolin-1-yl)methyl)benzyl)carbamate: The crude product of the previous reaction (500.00 mg, 1 Eq, 1.0493 mmol) was dissolved in CH2Cl2 (13 mL) and treated with benzoyl isocyanate (238.96 mg, 1.3 Eq, 1.36 mmol) and stirred at 45° C. for 3 h. The solvent was evaporated in vacuo and the residue was dissolved in anhydrous MeOH (5 mL), followed by the addition of sodium methoxide (1 eq), and was further stirred at 80° C. for 2 h. The solvent was removed under reduced pressure, partitioned between CH₂Cl₂ and water, the organic layer was dried over MgSO₄ and concentrated in vacuum. The LCMS showed two major peaks at 2.53 min and 2.72 min that corresponded to the acetylated and deacetylated product, respectively. The residue was used for the next step without further purification. HPLC rt=2.53; m/z=434.4 [M+H].

Step 5. (4-amino-1-(4-(aminomethyl)benzyl)-1H-imidazo[4,5-c]quinolin-2-yl)methanol: To a solution of crude the product from the previous step (450.0 mg, 1 Eq, 946.3 μmol) was added 20% TFA (10 mL) and the reaction was stirred at ambient temperature for 1 h. The solvent was evaporated under reduced pressure to afford a solid. The crude was purified on HPLC, yielding 45 mg of the title compound. HPLC=0.32 mg; m/z=334 [M+H].

### Example 5. Linker-Payloads Derived from Imiquimod

### LP #1: Preparation of 6-(2,5-Dioxo-2,5-dihydro-1H-pyrrol-1-yl)-N-(1-(1-(1-isobutyl-1H-pyrrolo[3,2-c]quinolin-4-ylamino)-1-oxo-5-ureidopentan-2-ylamino)-3-methyl-1-oxobutan-2-yl)hexanamide [mcValCit-Imiquimod]

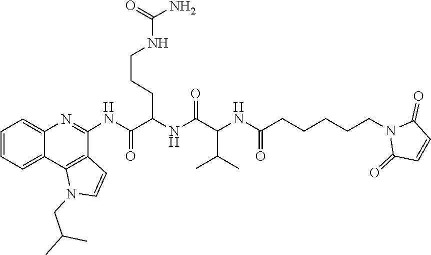 [SMILES: CC(C)Cn1ccc2c(NC(=O)C(CCCNC(N)=O)NC(=O)C(NC(=O)CCCCCN3C(=O)C=CC3=O)C(C)C)nc3ccccc3c21]

Imiquimod (10 mg, 42 μmol) and 2,6-dimethylpyridine (15 μL, 0.13 mmol) in DMF (2 mL) were stirred together for 15 min at rt before adding HOBt (8.3 mg, 54 μmol), HATU (19 mg, 50 μmol) and mcValCit-OH (21 mg, 46 μmol). The reaction mixture was further stirred for 12 h at rt. The crude reaction mixture was purified by reverse phase preparative HPLC using (10% to 95% ACN in water containing 0.05% TFA). Fractions containing the desired compound were dried under high vacuum to give 12.9 mg (44.5%) of title compound as a yellow oily substance. LC-MS (Protocol A): m/z 690.4 [M+1]⁺; Retention time=2.32 min. ¹H NMR (400 MHz, DMSO) δ_{H}/ppm ¹H NMR (400 MHz, DMSO) δ 8.79 (s, 1H), 8.51 (s, 1H), 8.49 (d, J=6.5 Hz, 1H), 8.43 (d, J=8.4 Hz, 2H), 7.86 (ddd, J=14.1, 12.0, 5.7 Hz, 4H), 6.99 (s, 2H), 4.89 (s, 1H), 4.61 (d, J=7.5 Hz, 2H), 4.50 (d, J=7.5 Hz, 1H), 4.29-4.22 (m, 1H), 3.37 (t, J=7.0 Hz, 3H), 3.04 (dd, J=10.9, 6.8 Hz, 2H), 2.94 (s, 1H), 2.23-2.08 (m, 5H), 1.53-1.40 (m, 10H), 1.18 (dd, J=13.8, 9.4 Hz, 4H), 0.95 (t, J=6.5 Hz, 9H), 0.90 (d, J=6.8 Hz, 4H), 0.87-0.81 (m, 8H).

### LP #2: 6-(2,5-dioxo-2,5-dihydro-1H-pyrrol-1-yl)-N-(1-isobutyl-1H-pyrrolo[3,2-c]quinolin-4-yl)hexanamide [mc-Imiquimod]

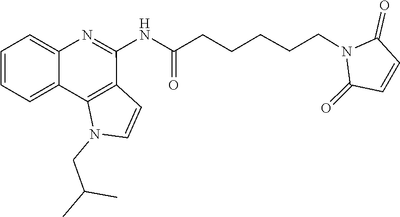

To a solution of 6-(2,5-dioxo-2,5-dihydro-1H-pyrrol-1-yl)hexanoic acid (15.0 mg, 71.0 μmol) in DMA (400 μL) was added HOBt (15.2 mg, 99.4 μmol), HATU (32.4 mg, 85.2 μmol) and 2,6-dimethylpyridine (24.7 μL, 213 μmol). After stirring for 15 min at rt, a solution of imiquimod (18.7 mg, 78.1 μmol) and 2,6-dimethylpyridine (24.7 μL, 213 μmol) in DMA (600 μL) was added dropwise, and the reaction mixture was stirred for 72 h at rt. The crude mixture was purified on prep-HPLC to obtain E131 (4.1 mg, 25%), LC-MS (Protocol A): m/z 764.5 [M+1]⁺; Retention time=2.78 min. ¹H NMR (400 MHz, DMSO) δ_{H}/ppm 8.78 (s, 1H), 8.44 (d, J=9.9 Hz, 2H), 7.92 (t, J=7.3 Hz, 1H), 7.85 (t, J=7.7 Hz, 1H), 7.01 (s, 2H), 4.62 (d, J=7.5 Hz, 3H), 3.43 (t, J=7.1 Hz, 2H), 2.79 (t, J=7.3 Hz, 2H), 2.21 (dt, J=14.9, 7.2 Hz, 1H), 1.76-1.66 (m, 2H), 1.56 (dt, J=14.9, 7.3 Hz, 2H), 1.40-1.31 (m, 2H), 0.97 (d, J=6.6 Hz, 6H), 0.94 (s, 1H).

### Example 6. Linker-Payloads Derived from Resiquimod (R848)

### LP #3. 4. 6-(2,5-Dioxo-2,5-dihydro-1H-pyrrol-1-yl)-N-(1-(1-(2-(ethoxymethyl)-1-(2-hydroxy-2-methylpropyl)-1H-pyrrolo[3,2-c]quinolin-4-ylamino)-1-oxo-5-ureidopentan-2-ylamino)-3-methyl-1-oxobutan-2-yl)hexanamide [mcValCit-Resiquimod]

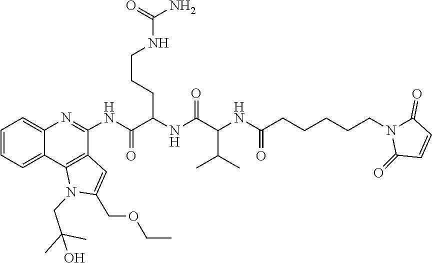 [SMILES: CCOCc1cc2c(NC(=O)C(CCCNC(N)=O)NC(=O)C(NC(=O)CCCCCN3C(=O)C=CC3=O)C(C)C)nc3ccccc3c2n1CC(C)(C)O]

DMA (0.5 mL) was added to a vial containing resiquimod (15 mg, 48 μmol) and HOBt (9.5 mg, 62 μmol). 2,6-Dimethylpyridine (17 μL, 0.14 mmol) was added with a syringe. The reaction mixture was stirred for 12 h at rt and crude product was purified by a reverse phase preparative HPLC using (10% acetonitrile/90% H₂O for 5 minutes, then 10% acetonitrile to 95% acetonitrile in H₂O over 10 minutes, each solvent containing 0.05% TFA), yielding 15.8 mg (42.7%) of the title compound as a yellowish oil. LC-MS (Protocol A): m/z 764.5 [M+1]⁺; Retention time=2.33 min. ¹H NMR (400 MHz, DMSO) δ_{H}/ppm 8.76 (d, J=8.4 Hz, 1H), 8.62-8.44 (m, 1H), 8.40 (d, J=8.4 Hz, 1H), 7.82 (ddd, J=22.6, 15.6, 7.9 Hz, 4H), 7.00 (s, 2H), 4.95 (s, 1H), 4.39-4.10 (m, 2H), 3.58 (dq, J=14.0, 7.0 Hz, 3H), 3.41-3.28 (m, 3H), 3.11-3.01 (m, 2H), 2.96 (d, J=4.6 Hz, 1H), 2.22-2.10 (m, 3H), 1.49 (dt, J=14.4, 7.2 Hz, 7H), 1.18 (dd, J=14.2, 7.3 Hz, 13H), 0.91 (d, J=6.7 Hz, 3H), 0.84 (dt, J=6.8, 5.6 Hz, 8H).

### LP #4: 4-(2-(2-(6-(2,5-Dioxo-2,5-dihydro-1H-pyrrol-1-yl)hexanamido)-3-methylbutanamido)-5-ureidopentanamido)benzyl 2-(ethoxymethyl)-1-(2-hydroxy-2-methylpropyl)-1H-imidazo[4,5-c]quinolin-4-ylcarbamate [mcValCitPABC-Resiquimod]

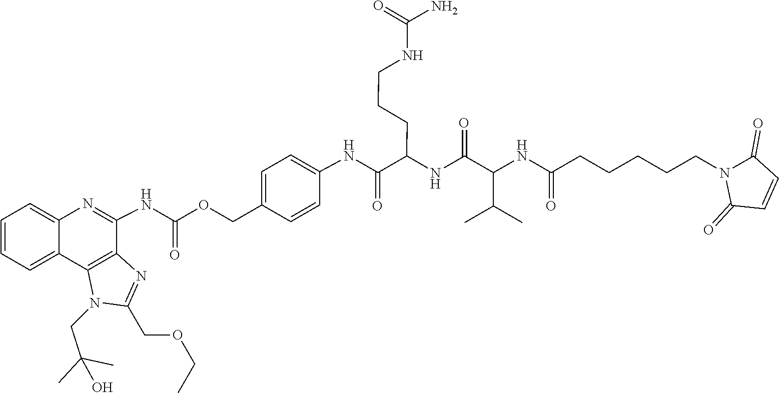 [SMILES: CCOCc1nc2c(NC(=O)OCc3ccc(NC(=O)C(CCCNC(N)=O)NC(=O)C(NC(=O)CCCCCN4C(=O)C=CC4=O)C(C)C)cc3)nc3ccccc3c2n1CC(C)(C)O]

To a solution of resiquimod (20 mg, 64 μmol) in DMF (300 μL) was added 2,6-dimethylpyridine (22 μL, 0.19 mmol) using a glass syringe. The reaction was then stirred at rt for 15 min. Thereafter, a solution of HOBt (12 mg, 76 μmol) and mcValCit-PAB-PNP (40 mg, 70 μmol) in DMF (300 μL) was added using a glass syringe and the reaction mixture was stirred overnight at rt. Water (5 mL) was added and the product was extracted with EtOAc (3×5 mL). The organic layer was dried over anhydrous MgSO₄. The organic fraction was evaporated at a reduced pressure, then the crude extract was dissolved in DMA (500 μL) and purified by a reverse phase preparative HPLC using (10% acetonitrile/90% H₂O for 5 minutes, then 10% acetonitrile to 95% acetonitrile in H₂O over 10 minutes, each solvent containing 0.05% TFA) to produce 6.5 mg (11.2%) of the title compound as a white solid. LC-MS (Protocol A): m/z 913.5 [M+1]⁺; Retention time=2.88 min. ¹H NMR (400 MHz, DMSO) δ_{H}/ppm 10.02 (s, 1H), 8.65 (d, J=8.4 Hz, 1H), 8.18-8.02 (m, 2H), 7.80 (d, J=8.7 Hz, 2H), 7.65 (d, J=8.6 Hz, 2H), 7.44 (d, J=8.6 Hz, 1H), 7.00 (s, 2H), 5.25 (s, 1H), 4.39 (dd, J=16.2, 9.1 Hz, 2H), 4.24-4.13 (m, 1H), 3.56 (q, J=7.0 Hz, 2H), 3.37 (t, J=7.0 Hz, 2H), 2.95 (s, 4H), 2.79 (s, 5H), 2.20-2.06 (m, 3H), 1.96 (s, 6H), 1.48 (dt, J=15.0, 7.3 Hz, 6H), 1.18 (dt, J=14.0, 7.6 Hz, 10H), 0.84 (dd, J=12.2, 6.8 Hz, 7H).

### LP #5: 6-(2,5-dioxo-2,5-dihydro-1H-pyrrol-1-yl)-N-(2-(ethoxymethyl)-1-(2-hydroxy-2-methylpropyl)-1H-imidazo[4,5-c]quinolin-4-yl)hexanamide [mc-resiquimod]

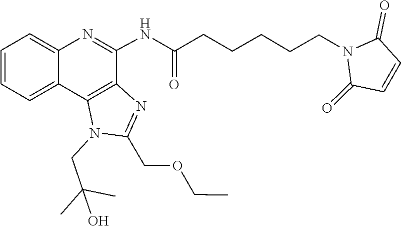 [SMILES: CCOCc1nc2c(NC(=O)CCCCCN3C(=O)C=CC3=O)nc3ccccc3c2n1CC(C)(C)O]

To a solution of 6-(2,5-dioxo-2,5-dihydro-1H-pyrrol-1-yl)hexanoic acid (13.0 mg, 61.5 μmol) in DMA (400 μL) was added 2,6-dimethylpyridine (21.4 μL, 185 μmol) and HOBt (13.2 mg, 86.2 μmol). This mixture was stirred for 15 min at rt and treated with a solution of resiquimod (21.3 mg, 67.7 μmol) and 2,6-dimethylpyridine (21.4 μL, 185 μmol) in DMA (300 μL). After 72 h at rt, the crude mixture was purified on prep-HPLC, producing 8.6 mg (27.6%) of E130 as a white solid, LC-MS (Protocol A): m/z 764.5 [M+1]⁺; Retention time=2.80 min. ¹H NMR (400 MHz, DMSO) δ_{H}/ppm 8.74 (d, J=8.3 Hz, 1H), 8.37 (d, J=8.4 Hz, 1H), 7.86 (t, J=7.5 Hz, 1H), 7.76 (t, J=7.7 Hz, 1H), 7.01 (s, 2H), 4.85-4.79 (m, 1H), 3.58 (dd, J=14.0, 7.0 Hz, 6H), 3.42 (t, J=7.0 Hz, 3H), 2.80 (t, J=7.3 Hz, 2H), 1.75-1.66 (m, 2H), 1.56 (dt, J=14.8, 7.3 Hz, 3H), 1.39-1.29 (m, 3H), 1.21 (s, 1H), 1.16 (t, J=7.0 Hz, 5H).

### Example 7. Linker-Payloads Derived from 1-(4-(aminomethyl)benzyl)-2-butyl-1H-imidazo[4,5-c]quinolin-4-amine (E66)

### LP #6: 4-(2-(2-(6-(2,5-Dioxo-2,5-dihydro-1H-pyrrol-1-yl)hexanamido)-3-methylbutanamido)-5-ureidopentanamido)benzyl 4-((4-amino-2-butyl-1H-imidazo[4,5-c]quinolin-1-yl)methyl)benzylcarbamate [mcValCitPABC-E66]

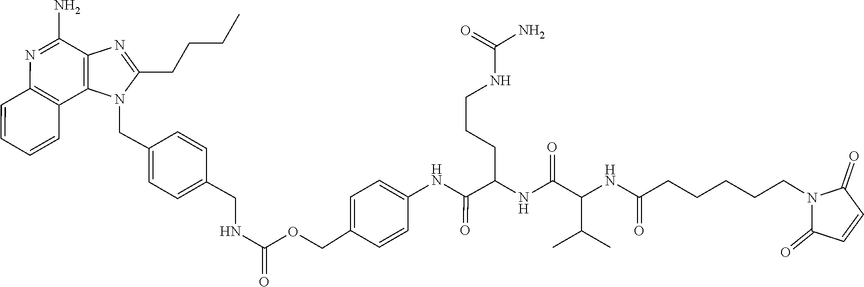 [SMILES: CCCCc1nc2c(N)nc3ccccc3c2n1Cc1ccc(CNC(=O)OCc2ccc(NC(=O)C(CCCNC(N)=O)NC(=O)C(NC(=O)CCCCCN3C(=O)C=CC3=O)C(C)C)cc2)cc1]

2,6-Dimethylpyridine (7.9 μL, 68 μmol) was added to a solution of 1-(4-(aminomethyl)benzyl)-2-butyl-1H-imidazo[4,5-c]quinolin-4-amine (Example 1) (8.2 mg, 23 μmol) in DMA (400 μL) and stirred for 15 min at 25° C. HOBt (4.2 mg, 1.2 Eq, 27 μmol). mcValCitPAB-PNP (14 mg, 25 μmol) in DMA (200 μL) was added, and the reaction mixture was stirred overnight at rt. Saturated solution of NaHCO₃ (3 mL) was added and the product was extracted with CH₂Cl₂ (3×5 mL). The organic fraction was dried over anhydrous MgSO₄, and the solvent evaporated in vacuo. The crude extract was dissolved in DMA (500 μL) and purified by a reverse phase preparative HPLC using (10% acetonitrile/90% H₂O for 5 minutes, then 10% acetonitrile to 95% acetonitrile in H₂O over 10 minutes, each solvent containing 0.05% TFA) to yield white solid (16.4 mg, 74.6%). LC-MS (Protocol A): m z 958.5 [M+1]⁺; Retention time=2.55 min. ¹H NMR (400 MHz, DMSO) δ_{H}/ppm 10.06 (s, 1H), 9.92 (dd, J=76.6, 35.4 Hz, 2H), 8.07 (dd, J=14.4, 7.5 Hz, 3H), 8.02 (d, J=8.4 Hz, 1H), 7.81 (d, J=8.6 Hz, 4H), 7.61 (d, J=8.5 Hz, 5H), 7.58-7.50 (m, 4H), 7.45-7.35 (m, 5H), 7.32 (d, J=8.5 Hz, 2H), 7.23 (d, J=8.5 Hz, 1H), 7.00 (s, 4H), 6.32-5.59 (m, 22H), 5.53 (s, 2H), 5.06 (d, J=10.3 Hz, 2H), 4.43 (s, 1H), 4.42-4.33 (m, 3H), 4.19 (t, J=7.7 Hz, 3H), 3.37 (t, J=7.0 Hz, 6H), 3.08-2.90 (m, 7H), 2.25-2.06 (m, 7H), 2.06-1.86 (m, 3H), 1.71 (dt, J=14.4, 7.2 Hz, 4H), 1.59 (dd, J=13.5, 4.3 Hz, 3H), 1.48 (dt, J=14.7, 7.3 Hz, 14H), 1.37 (dd, J=14.9, 7.4 Hz, 5H), 1.24-1.13 (m, 6H), 0.84 (dd, J=12.4, 6.7 Hz, 19H).

### LP #7: N-(4-((4-amino-2-butyl-1H-imidazo[4,5-c]quinolin-1-yl)methyl)benzyl)-6-(2,5-dioxo-2,5-dihydro-1H-pyrrol-1-yl)hexanamide. [mc-E66]

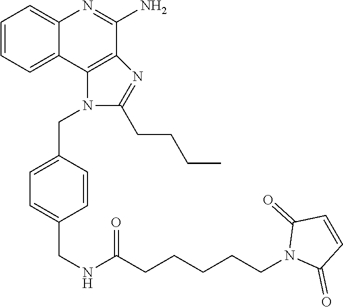 [SMILES: CCCCc1nc2c(N)nc3ccccc3c2n1Cc1ccc(CNC(=O)CCCCCN2C(=O)C=CC2=O)cc1]

A solution of E66 [1-(4-(aminomethyl)benzyl)-2-butyl-1H-imidazo[4,5-c]quinolin-4-amine (4.40 mg, 12.2 μmol)], 6-(2,5-dioxo-2,5-dihydro-1H-pyrrol-1-yl)hexanoic acid (3.10 mg, 14.7 μmol), HATU (6.05 mg, 1.3 Eq, 15.9 μmol), 1H-benzo[d][1,2,3]triazol-1-ol hydrate (2.62 mg, 1.4 Eq, 17.1 μmol) and 2,6-dimethylpyridine (3.93 mg, 4.25 μL, 3 Eq, 36.7 μmol) was stirred at rt for 1.5 hr. The crude mixture was purified on HPLC (to obtain the desired product (1.6 mg, 36.4%). LC-MS (Protocol A): m/z 552.68 [M+1]⁺; Retention time=3.24 min. ¹H NMR (400 MHz, DMSO) δ_{H}/ppm 13.65 (s, 1H), 8.52 (dd, J=11.5, 7.9 Hz, 1H), 7.79 (d, J=8.2 Hz, 1H), 7.69 (dd, J=7.4, 3.5 Hz, 2H), 7.51 (dd, J=14.9, 7.0 Hz, 1H), 7.01 (dd, J=6.3, 3.1 Hz, 1H), 6.99 (s, 2H), 6.95 (s, 1H), 3.72 (dd, J=16.4, 8.7 Hz, 1H), 3.50 (dd, J=14.2, 7.1 Hz, 3H), 3.41-3.30 (m, 3H), 2.94 (s, 1H), 2.78 (s, 1H), 2.68 (s, 2H), 2.54 (s, 1H), 2.35 (t, J=7.4 Hz, 2H), 1.95 (s, 1H), 1.56-1.39 (m, 4H), 1.29-1.18 (m, 2H), 1.13 (t, J=7.1 Hz, 3H), 1.02 (t, J=7.1 Hz, 1H).

### LP #8: N-(1-(1-(4-((4-amino-2-butyl-1H-imidazo[4,5-c]quinolin-1-yl)methyl)benzylamino)-1-oxo-5-ureidopentan-2-ylamino)-3-methyl-1-oxobutan-2-yl)-6-(2,5-dioxo-2,5-dihydro-1H-pyrrol-1-yl)hexanamide [mcValCit-E66]

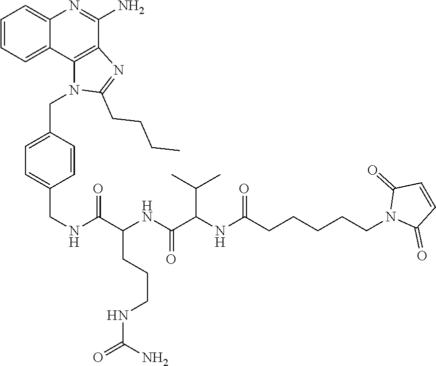 [SMILES: CCCCc1nc2c(N)nc3ccccc3c2n1Cc1ccc(CNC(=O)C(CCCNC(N)=O)NC(=O)C(NC(=O)CCCCCN2C(=O)C=CC2=O)C(C)C)cc1]

Step 1. A solution of E66 [1-(4-(aminomethyl)benzyl)-2-butyl-1H-imidazo[4,5-c]quinolin-4-amine, 10 mg, 1 Eq, 28 μmol], Boc-ValCit-OH (12 mg, 1.2 Eq, 33 μmol), HATU (14 mg, 1.3 Eq, 36 μmol), 2,6 lutidine (8.6 mg, 3 eq) and 1H-benzo[d][1,2,3]triazol-1-ol hydrate (6.0 mg, 1.4 Eq, 39 μmol) in DMA (500 μl) was stirred for 1 h at rt. The reaction was monitored by LCMS. The crude mixture was purified on HPLC to give 1.3 mg (m/z=716.9) of product as a white solid. The product was treated with 1 mL of 20% TFA in DCM for 1 h. The solvent was evaporated and the material was used in the next step without purification.

Step 2. To a solution of the product of step 1 (1.10 mg, 1 Eq, 1.79 μmol) in DMA (300 μl) was added 6-(2,5-dioxo-2,5-dihydro-1H-pyrrol-1-yl)hexanoic acid (453 μg, 1.2 Eq, 2.14 μmol), 2-(3H-[1,2,3]triazolo[4,5-b]pyridin-3-yl)-1,1,3,3-tetramethylisouronium hexafluorophosphate(V) (883 μg, 1.3 Eq, 2.32 μmol), and the reaction mixture was stirred for 1.5 h at rt. The crude mixture was purified on prep-HPLC to obtain the desired product, LC-MS (Protocol A): m/z 808.99 [M+1]⁺; Retention time=2.90 min. ¹H NMR (400 MHz, DMSO) δ_{H}/ppm 8.31 (dd, J=7.3, 4.6 Hz, 1H), 7.95 (d, J=7.7 Hz, 1H), 7.90 (d, J=7.9 Hz, 1H), 7.80 (d, J=7.5 Hz, 1H), 7.77-7.71 (m, 1H), 7.65-7.59 (m, 1H), 7.40 (d, J=2.1 Hz, 1H), 7.36 (d, J=8.7 Hz, 1H), 7.21-7.16 (m, 2H), 7.00 (s, 2H), 5.93 (s, 1H), 5.79 (d, J=1.8 Hz, 2H), 3.31 (d, J=3.2 Hz, 2H), 2.67 (dt, J=3.9, 1.9 Hz, 7H), 2.52 (d, J=1.7 Hz, 6H), 2.34-2.31 (m, 7H), 1.52-1.43 (m, 3H), 1.37 (dt, J=16.4, 7.3 Hz, 3H), 1.17 (d, J=7.5 Hz, 1H), 0.87 (t, J=7.3 Hz, 7H), 0.82 (d, J=2.4 Hz, 2H), 0.74 (d, J=6.7 Hz, 6H).

### Example 8. Linker-Payloads Derived from 1-(4-aminobenzyl)-2-butyl-1H-imidazo[4,5-c]quinolin-4-amine (E104)

### LP #9. 4-(2-(2-(6-(2,5-dioxo-2,5-dihydro-1H-pyrrol-1-yl)hexanamido)-3-methylbutanamido)-5-ureidopentanamido)benzyl 4-((4-amino-2-butyl-1H-imidazo[4,5-c]quinolin-1-yl)methyl)phenylcarbamate [mcValCitPABC-E104]

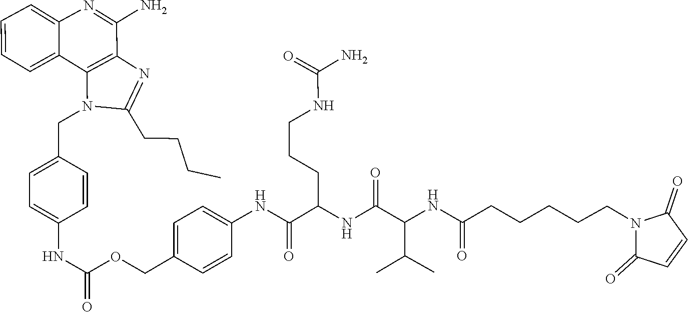 [SMILES: CCCCc1nc2c(N)nc3ccccc3c2n1Cc1ccc(NC(=O)OCc2ccc(NC(=O)C(CCCNC(N)=O)NC(=O)C(NC(=O)CCCCCN3C(=O)C=CC3=O)C(C)C)cc2)cc1]

To a solution of 1-(4-aminobenzyl)-2-butyl-1H-imidazo[4,5-c]quinolin-4-amine (E104, Example 2) (9.1 mg, 26 μmol) in DMA (500 μL) was added mcValCit-PAB-PNP (23 mg, 32 μmol), 2,6-dimethylpyridine (9.2 μL, 79 μmol) and HOBt (5.6 mg, 37 μmol). The mixture was stirred overnight at rt. The crude mixture was purified by prep-HPLC to give an amber-colored sticky solid (36.2 mg, 60.3%). LC-MS (Protocol A): m/z 944.1 [M+1]⁺; Retention time=3.27 min. ¹H NMR (400 MHz, DMSO) δ_{H}/ppm 9.97 (s, 1H), 7.79 (d, J=1.2 Hz, 1H), 7.59 (d, J=8.8 Hz, 1H), 7.40 (d, J=9.2 Hz, 1H), 7.32 (d, J=8.7 Hz, 1H), 7.00 (s, 1H), 6.99 (s, 2H), 6.97 (d, J=6.6 Hz, 1H), 6.51 (s, 2H), 5.39 (s, 1H), 5.04 (s, 1H), 2.77 (d, J=2.7 Hz, 2H), 2.69-2.65 (m, 11H), 2.33 (dt, J=3.9, 2.1 Hz, 9H), 2.08 (s, 3H), 1.95 (s, 4H), 1.24 (s, 3H), 0.89-0.86 (m, 2H), 0.85 (t, J=1.8 Hz, 2H), 0.84-0.80 (m, 2H).

### LP #10: N-(1-(1-(4-((4-amino-2-butyl-1H-imidazo[4,5-c]quinolin-1-yl)methyl)phenylamino)-1-oxo-5-ureidopentan-2-ylamino)-3-methyl-1-oxobutan-2-yl)-6-(2,5-dioxo-2,5-dihydro-1H-pyrrol-1-yl)hexanamide [mcValCit-E104]

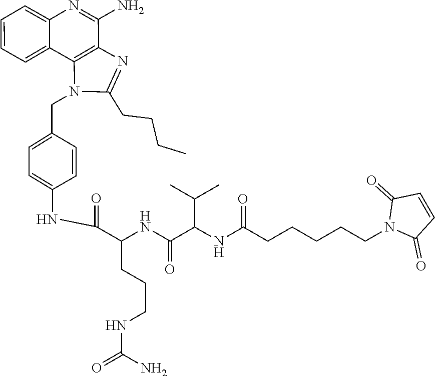 [SMILES: CCCCc1nc2c(N)nc3ccccc3c2n1Cc1ccc(NC(=O)C(CCCNC(N)=O)NC(=O)C(NC(=O)CCCCCN2C(=O)C=CC2=O)C(C)C)cc1]

Step 1. A solution of E104 [1-(4-aminobenzyl)-2-butyl-1H-imidazo[4,5-c]quinolin-4-amine, 9.20 mg, 1 Eq, 26.6 μmol] in 500 μL DMA was treated with Boc-ValCit-OH (13.0 mg, 1.3 Eq, 34.6 μmol), 1H-benzo[d][1,2,3]triazol-1-ol hydrate (6.12 mg, 1.5 Eq, 39.9 μmol), HATU (14.2 mg, 1.4 eq), and 2,6-lutidine (8.5 mg, 3 eq). The reaction was stirred for 1 h at rt and monitored by LCMS. Complete conversion was noted after 1 h. The crude material was purified by HPLC to give 4.2 mg (22.5%) of the desired product as a white solid. HPLC rt=3.03; m/z=702.5 [M+H].

Step 2. The product of step 1 was treated with 1 mL of 20% TFA in DCM. After 1 h, LCMS indicated that the reaction was complete. The solvent was evaporated to give a crude product that was immediately treated with 6-(2,5-dioxo-2,5-dihydro-1H-pyrrol-1-yl)hexanoic acid (1.3 mg, 1.2 Eq, 6.4 μmol), HATU (2.6 mg, 1.3 Eq, 6.9 μmol), 1H-benzo[d][1,2,3]triazol-1-ol hydrate (1.1 mg, 1.4 Eq, 7.4 μmol) and 2,6-dimethylpyridine (1.7 mg, 1.8 μL, 3 Eq, 16 μmol) in DMA (300 μL). After stirring at rt for 1 h, the product mixture was purified by HPLC giving 1.1 mg of the title compound. HPLC rt=2.97; m/z 795.5 [M+H].

### LP #11: N-(4-((4-amino-2-butyl-1H-imidazo[4,5-c]quinolin-1-yl)methyl)phenyl)-6-(2,5-dioxo-2,5-dihydro-1H-pyrrol-1-yl)hexanamide [mc-E104]

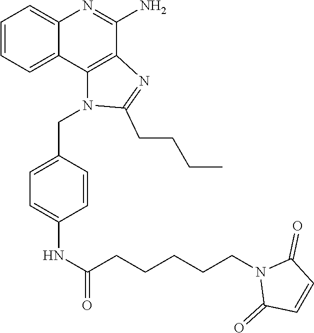 [SMILES: CCCCc1nc2c(N)nc3ccccc3c2n1Cc1ccc(NC(=O)CCCCCN2C(=O)C=CC2=O)cc1]

A solution of 1-(4-aminobenzyl)-2-butyl-1H-imidazo[4,5-c]quinolin-4-amine (Example 2) (10.0 mg, 28.95 μmol)], 6-(2,5-dioxo-2,5-dihydro-1H-pyrrol-1-yl)hexanoic acid (7.337 mg, 34.74 μmol), HATU (14.31 mg, 37.63 μmol), 1H-benzo[d][1,2,3]triazol-1-ol hydrate (6.206 mg, 40.53 μmol) and 2,6-dimethylpyridine (9.3 mg, 10.1 μL, 86.8 μmol) was stirred at rt for 1.5 hr. The crude mixture was purified on HPLC to obtain the desired product (11.2 mg, 71.8%) as a faint yellow solid. LC-MS (Protocol A): m/z 538.6 [M+1]⁺; Retention time=3.47 min. ¹H NMR (400 MHz, DMSO) δ_{H}/ppm 9.86 (s, 1H), 7.96 (d, J=8.1 Hz, 1H), 7.79 (d, J=8.2 Hz, 1H), 7.65-7.60 (m, 1H), 7.52 (dd, J=9.0, 2.9 Hz, 3H), 7.40-7.35 (m, 1H), 7.01 (d, J=4.0 Hz, 2H), 7.00-6.98 (m, 2H), 6.97 (s, 2H), 6.96 (d, J=2.3 Hz, 1H), 5.89 (s, 1H), 3.37 (dd, J=8.8, 5.3 Hz, 3H), 2.98 (d, J=7.5 Hz, 1H), 2.95 (d, J=3.2 Hz, 1H), 2.24 (dd, J=12.8, 5.4 Hz, 2H), 1.72 (ddd, J=12.2, 7.4, 3.6 Hz, 2H), 1.58-1.46 (m, 3H), 1.38 (dt, J=14.5, 7.5 Hz, 2H), 1.27-1.17 (m, 2H), 0.85 (t, J=3.7 Hz, 2H).

### Example 9. Linker-Payloads Derived from Gardiquimod

### LP #12: 4-(2-(2-(6-(2,5-dioxo-2,5-dihydro-1H-pyrrol-1-yl)hexanamido)-3-methylbutanamido)-5-ureidopentanamido)benzyl (4-amino-1-(2-hydroxy-2-methylpropyl)-1H-imidazo[4,5-c]quinolin-2-yl)methyl(ethyl)carbamate [mcValCitPABC-gardiquimod]

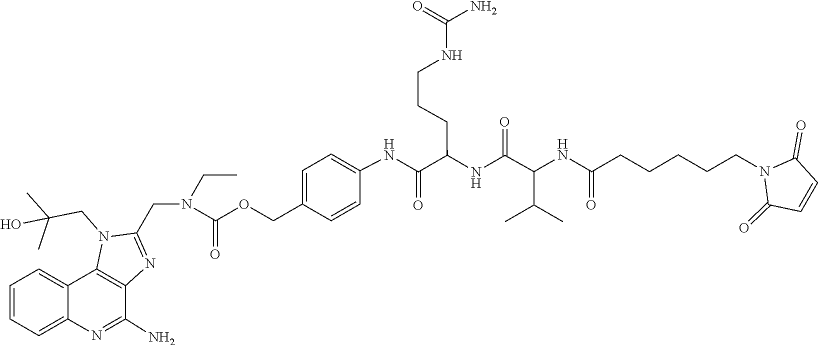 [SMILES: CCN(Cc1nc2c(N)nc3ccccc3c2n1CC(C)(C)O)C(=O)OCc1ccc(NC(=O)C(CCCNC(N)=O)NC(=O)C(NC(=O)CCCCCN2C(=O)C=CC2=O)C(C)C)cc1]

To a solution of 1-(4-amino-2-((ethylamino)methyl)-1H-imidazo[4,5-c]quinolin-1-yl)-2-methylpropan-2-ol bis(2,2,2-trifluoroacetate) (10 mg, 18 μmol) in DMA (500 μL) was added 2,6-dimethylpyridine (13 μL, 0.11 mmol) and stirred for 10 min at rt. Then, 1H-benzo[d][1,2,3]triazol-1-ol hydrate (4.0 mg, 26 μmol) and mcValCitPAB-PNP (16 mg, 22 μmol) were added, and the reaction was further stirred at rt for 12 h. The crude mixture was purified on prep-HPLC to give an amber-colored sticky solid (7.4 mg, 43.5%). LC-MS (Protocol A): m/z 958.5 [M+1]⁺; Retention time=2.74 min. ¹H NMR (400 MHz, DMSO) δ_{H}/ppm 9.94 (d, J=34.6 Hz, 1H), 8.88 (s, 2H), 8.45 (d, J=57.6 Hz, 1H), 8.11 (d, J=9.2 Hz, 1H), 8.06 (d, J=7.5 Hz, 1H), 7.79 (dd, J=8.4, 3.6 Hz, 2H), 7.68 (t, J=7.7 Hz, 1H), 7.51 (t, J=7.7 Hz, 1H), 7.43 (s, 1H), 7.31 (s, 1H), 7.16 (s, 1H), 6.99 (s, 2H), 6.93 (d, J=9.2 Hz, 1H), 6.04 (s, 1H), 5.01 (d, J=38.4 Hz, 2H), 4.38 (dd, J=13.4, 8.1 Hz, 3H), 4.23-4.14 (m, 10H), 3.36 (t, J=7.1 Hz, 3H), 3.06-2.91 (m, 2H), 2.22-2.07 (m, 2H), 2.01-1.91 (m, 1H), 1.52-1.42 (m, 5H), 1.18 (dt, J=15.4, 7.8 Hz, 5H), 1.08 (s, 7H), 0.84 (dd, J=11.7, 6.8 Hz, 7H).

### LP #13: N-((4-amino-1-(2-hydroxy-2-methylpropyl)-1H-imidazo[4,5-c]quinolin-2-yl)methyl)-6-(2,5-dioxo-2,5-dihydro-1H-pyrrol-1-yl)-N-ethylhexanamide [mc-Gardiquimod]

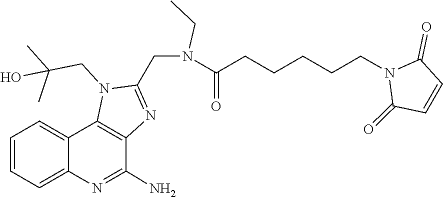 [SMILES: CCN(Cc1nc2c(N)nc3ccccc3c2n1CC(C)(C)O)C(=O)CCCCCN1C(=O)C=CC1=O]

To a solution of 6-(2,5-dioxo-2,5-dihydro-1H-pyrrol-1-yl)hexanoic acid (4.0 mg, 19 μmol) in DMA (400 μL) was added HATU (8.6 mg, 23 μmol), HOBt (4.1 mg, 27 μmol) and 2,6-dimethylpyridine (6.6 μL, 57 μmol). After stirring for 15 min at rt, a solution of gardiquimod trifluoroacetate (10 mg, 19 μmol) and 2,6-dimethylpyridine (6.1 mg, 6.6 μL, 3 Eq, 57 μmol) in DMA (600 μL) was added dropwise, and the reaction mixture was stirred for 12 h at rt. The crude mixture was purified on prep-HPLC to obtain 6.6 mg (68.8%) as a white solid, LC-MS (Protocol A): m/z 506.6 [M+1]⁺; Retention time=2.47 min. ¹H NMR (400 MHz, DMSO) δ_{H}/ppm 13.65 (s, 1H), 8.52 (dd, J=11.5, 7.9 Hz, 1H), 7.80 (t, J=8.2 Hz, 1H), 7.71-7.65 (m, 2H), 7.51 (dd, J=15.0, 7.0 Hz, 1H), 7.01 (dd, J=6.2, 3.0 Hz, 1H), 6.99 (s, 2H), 3.72 (dd, J=16.4, 8.7 Hz, 1H), 3.50 (dd, J=14.2, 7.2 Hz, 2H), 3.40-3.30 (m, 2H), 2.86 (d, J=64.1 Hz, 1H), 2.71-2.64 (m, 3H), 2.54 (s, 1H), 2.35 (t, J=7.4 Hz, 1H), 1.50 (ddd, J=12.2, 11.4, 6.1 Hz, 2H), 1.24 (tdd, J=16.0, 10.1, 6.2 Hz, 2H), 1.13 (t, J=7.1 Hz, 2H), 1.02 (t, J=7.1 Hz, 1H).

### Example 10. Linker-Payloads Derived from 4-amino-1-(4-(aminomethyl)benzyl)-1H-imidazo[4,5-c]quinolin-2-yl)methanol (E75)

### LP #14: 4-(2-(2-(6-(2,5-dioxo-2,5-dihydro-1H-pyrrol-1-yl)hexanamido)-3-methylbutanamido)-5-ureidopentanamido)benzyl 4-((4-amino-2-(hydroxymethyl)-1H-imidazo[4,5-c]quinolin-1-yl)methyl)benzylcarbamate [mcValCitPABC-E75]

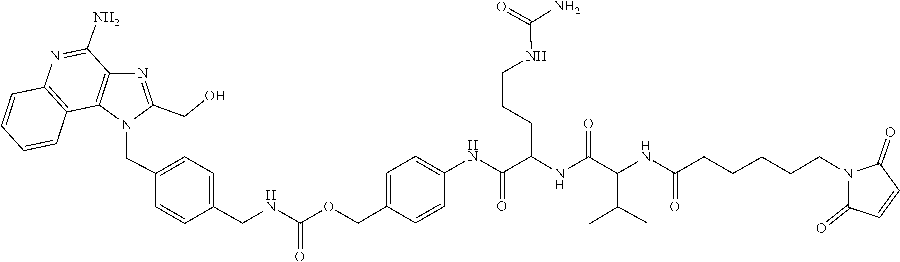 [SMILES: CC(C)C(NC(=O)CCCCCN1C(=O)C=CC1=O)C(=O)NC(CCCNC(N)=O)C(=O)Nc1ccc(COC(=O)NCc2ccc(Cn3c(CO)nc4c(N)nc5ccccc5c43)cc2)cc1]

To a solution of (4-amino-1-(4-(aminomethyl)benzyl)-1H-imidazo[4,5-c]quinolin-2-yl)methanol (Example 4) (17 mg, 51 μmol) in DMA (500 μL) was added 2,6-dimethylpyridine (7.6 mg, 71 μmol) and the mixture was stirred at rt for 15 min. 1H-benzo[d][1,2,3]triazol-1-ol hydrate (23 mg, 25 μL, 0.15 mmol) and 4-(2-(2-(6-(2,5-dioxo-2,5-dihydro-1H-pyrrol-1-yl)hexanamido)-3-methylbutanamido)-5-ureidopentanamido)benzyl (4-nitrophenyl) carbonate (mcValCitPAB-PNP) (49 mg, 66 μmol) were added and the mixture was stirred overnight at rt. The title product was purified by HPLC to give a white solid (5.0 mg, 10%). LC-MS (Protocol A): m/z 932.05 [M+1]⁺; Retention time=2.86 min. ¹H NMR (400 MHz, DMSO) δ_{H}/ppm 9.97 (s, 1H), 7.79 (dd, J=8.5, 4.2 Hz, 2H), 7.58 (d, J=8.7 Hz, 1H), 7.26 (d, J=8.7 Hz, 1H), 7.20 (d, J=8.2 Hz, 2H), 7.08 (d, J=8.3 Hz, 2H), 7.00 (s, 2H), 6.99-6.96 (m, 1H), 6.02 (s, 1H), 4.93 (s, 1H), 4.82 (d, J=2.1 Hz, 1H), 4.38 (dd, J=7.9, 2.5 Hz, 1H), 4.17 (dd, J=23.8, 6.5 Hz, 2H), 4.00 (d, J=53.7 Hz, 1H), 3.47 (d, J=14.5 Hz, 3H), 3.23 (d, J=2.3 Hz, 2H), 2.87 (d, J=64.1 Hz, 2H), 2.68 (dt, J=4.1, 2.1 Hz, 4H), 2.33 (dt, J=4.0, 2.0 Hz, 3H), 2.09-2.09 (m, 2H), 1.51 (d, J=7.6 Hz, 1H), 1.47 (dd, J=5.2, 2.5 Hz, 2H), 1.20 (d, J=35.8 Hz, 3H), 0.86 (d, J=6.8 Hz, 3H), 0.83 (d, J=6.7 Hz, 3H).

### LP #15: N-(4-((4-amino-2-(hydroxymethyl)-1H-imidazo[4,5-c]quinolin-1-yl)methyl)benzyl)-6-(2,5-dioxo-2,5-dihydro-1H-pyrrol-1-yl)hexanamide [mc-E75]

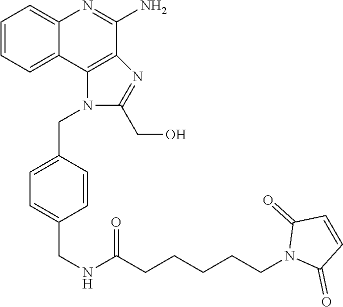 [SMILES: Nc1nc2ccccc2c2c1nc(CO)n2Cc1ccc(CNC(=O)CCCCCN2C(=O)C=CC2=O)cc1]

To a solution of (4-amino-1-(4-(aminomethyl)benzyl)-1H-imidazo[4,5-c]quinolin-2-yl)methanol (example 4) (17.00 mg, 50.99 μmol) in DMA (500 μL) were added 6-(2,5-dioxo-2,5-dihydro-1H-pyrrol-1-yl)hexanoic acid (12.92 mg, 61.19 μmol), HATU (25.20 mg, 66.29 μmol), 1H-benzo[d][1,2,3]triazol-1-ol hydrate (10.93 mg, 71.39 μmol) and 2,6-dimethylpyridine (17.7 μL, 153.0 μmol). The reaction was stirred for 1 h at rt. The crude mixture was purified on HPLC to give a white solid product (2.3 mg); LC-MS (Protocol A): m/z 526.60 [M+1]⁺; Retention time=2.43 min. ¹H NMR (400 MHz, DMSO) δ_{H}/ppm 13.60 (s, 1H), 8.24 (t, J=6.1 Hz, 1H), 7.95 (d, J=8.3 Hz, 1H), 7.79 (d, J=8.3 Hz, 1H), 7.63 (t, J=7.8 Hz, 1H), 7.37 (t, J=7.7 Hz, 1H), 7.18 (d, J=8.2 Hz, 2H), 7.08 (d, J=8.2 Hz, 2H), 7.02-7.00 (m, 1H), 6.99 (s, 3H), 6.02 (s, 1H), 4.82 (s, 2H), 4.19 (d, J=6.0 Hz, 2H), 3.33 (t, J=7.0 Hz, 3H), 2.51 (d, J=3.8 Hz, 1H), 2.07 (t, J=7.4 Hz, 2H), 1.51-1.41 (m, 4H), 1.19-1.11 (m, 2H).

### Example 11. Linker-Payloads Derived from (4-amino-1-(4-aminobenzyl)-1H-imidazo[4,5-c]quinolin-2-yl)methanol [E136]

### LP #16: 4-(2-(2-(6-(2,5-dioxo-2,5-dihydro-1H-pyrrol-1-yl)hexanamido)-3-methylbutanamido)-5-ureidopentanamido)benzyl 4-((4-amino-2-(hydroxymethyl)-1H-imidazo[4,5-c]quinolin-1-yl)methyl)phenylcarbamate [mcValCitPABC-E136]

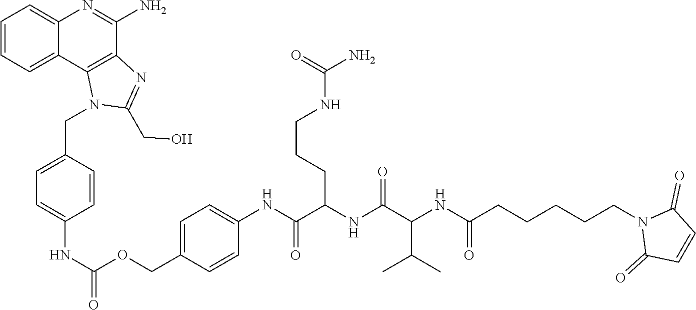 [SMILES: CC(C)C(NC(=O)CCCCCN1C(=O)C=CC1=O)C(=O)NC(CCCNC(N)=O)C(=O)Nc1ccc(COC(=O)Nc2ccc(Cn3c(CO)nc4c(N)nc5ccccc5c43)cc2)cc1]

To a solution of crude 4-amino-1-(4-aminobenzyl)-1H-imidazo[4,5-c]quinolin-2-yl)methanol (20.00 mg, 62.62 μmol) in DMA (500 μL) was added 2,6-dimethylpyridine (8.724 mg, 81.41 μmol) and stirred at rt for 15 min. Then, 1H-benzo[d][1,2,3]triazol-1-ol hydrate (28.77 mg, 187.9 μmol) and 4-(2-(2-(6-(2,5-dioxo-2,5-dihydro-1H-pyrrol-1-yl)hexanamido)-3-methylbutanamido)-5-ureidopentanamido)benzyl (4-nitrophenyl) carbonate (55.44 mg, 75.15 μmol) were added and stirred overnight at rt. The crude mixture was purified on HPLC to give 9.8 mg (17%) of the product as a white solid. LC-MS (Protocol A): m/z 918.03 [M+1]⁺; Retention time=2.74 min. ¹H NMR (400 MHz, DMSO) δ_{H}/ppm 10.00 (d, J=14.4 Hz, 5H), 8.07 (d, J=2.0 Hz, 3H), 8.05 (dd, J=4.5, 2.7 Hz, 6H), 7.85-7.74 (m, 9H), 7.62-7.59 (m, 11H), 7.58 (dd, J=4.5, 2.4 Hz, 14H), 7.43-7.37 (m, 7H), 7.36 (t, J=2.5 Hz, 4H), 7.34 (t, J=2.4 Hz, 5H), 7.31 (d, J=3.3 Hz, 3H), 7.28 (d, J=2.2 Hz, 4H), 7.03-6.98 (m, 43H), 5.81 (s, 4H), 4.98 (s, 4H), 4.40-4.35 (m, 3H), 4.19 (ddd, J=8.7, 6.6, 4.2 Hz, 8H), 2.85 (d, J=2.3 Hz, 11H), 2.68 (dt, J=3.9, 1.9 Hz, 8H), 2.34 (dt, J=6.0, 2.0 Hz, 8H), 1.50 (dd, J=7.6, 5.0 Hz, 8H), 1.47 (d, J=3.4 Hz, 2H), 1.45 (t, J=3.6 Hz, 3H), 1.25-1.13 (m, 5H), 0.86 (dt, J=6.7, 3.2 Hz, 17H), 0.83-0.79 (m, 11H).

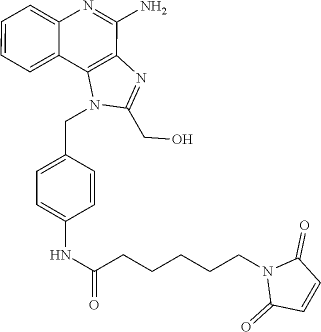 [SMILES: Nc1nc2ccccc2c2c1nc(CO)n2Cc1ccc(NC(=O)CCCCCN2C(=O)C=CC2=O)cc1]

### LP #17: N-(4-((4-amino-2-(hydroxymethyl)-1H-imidazo[4,5-c]quinolin-1-yl)methyl)phenyl)-6-(2,5-dioxo-2,5-dihydro-1H-pyrrol-1-yl)hexanamide [mc-E136]

To a solution of E136 [(4-amino-1-(4-aminobenzyl)-1H-imidazo[4,5-c]quinolin-2-yl)methanol, 50.0 mg, 1 Eq, 156.6 μmol] in DMA (500 μL) was added 6-(2,5-dioxo-2,5-dihydro-1H-pyrrol-1-yl)hexanoic acid (39.7 mg, 1.2 Eq, 187.9 μmol), HATU (77.39 mg, 1.3 Eq, 203.5 μmol), 1H-benzo[d][1,2,3]triazol-1-ol hydrate (33.57 mg, 1.4 Eq, 219.2 μmol) and 2,6-dimethylpyridine (50.33 mg, 54.4 μL, 3 Eq, 469.7 μmol). The reaction was stirred at rt for 1 h. The crude mixture was purified on HPLC to give 1.3 mg (1.6%) of the title product as a white solid. HPLC rt=2.87; m/z=513.3 [M+H].

### Example 11A

Preparation of various TLR agonists that are poised for attachment of the linker and antibody are carried out in a variety of ways known to those skilled in the art. One such method is outlined in Scheme P1 (adapted from US 2014/0141033).

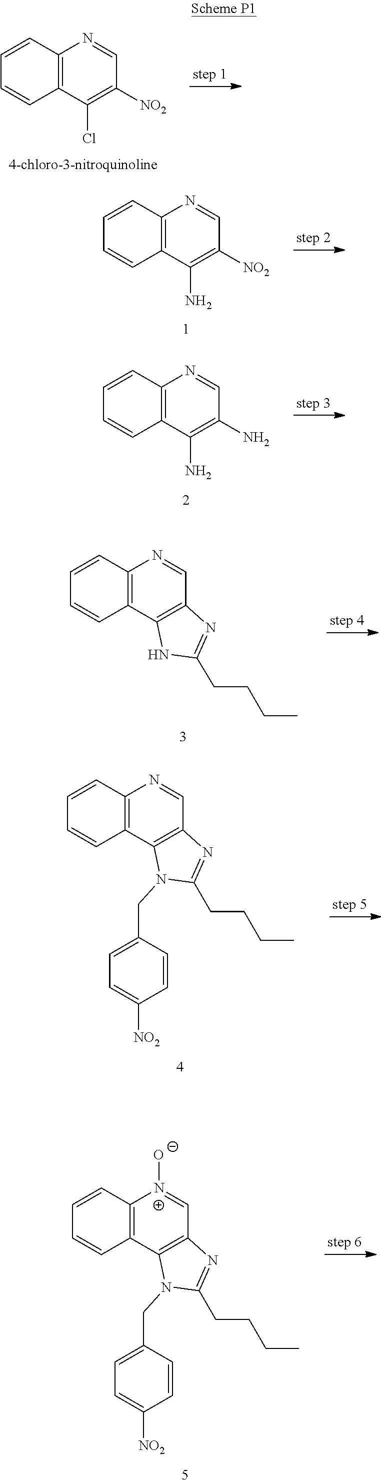 [SMILES: O=[N+]([O-])c1cnc2ccccc2c1Cl ; Nc1c([N+](=O)[O-])cnc2ccccc12 ; Nc1cnc2ccccc2c1N ; CCCCc1nc2cnc3ccccc3c2[nH]1 ; CCCCc1nc2cnc3ccccc3c2n1Cc1ccc([N+](=O)[O-])cc1 ; CCCCc1nc2c[n+]([O-])c3ccccc3c2n1Cc1ccc([N+](=O)[O-])cc1]

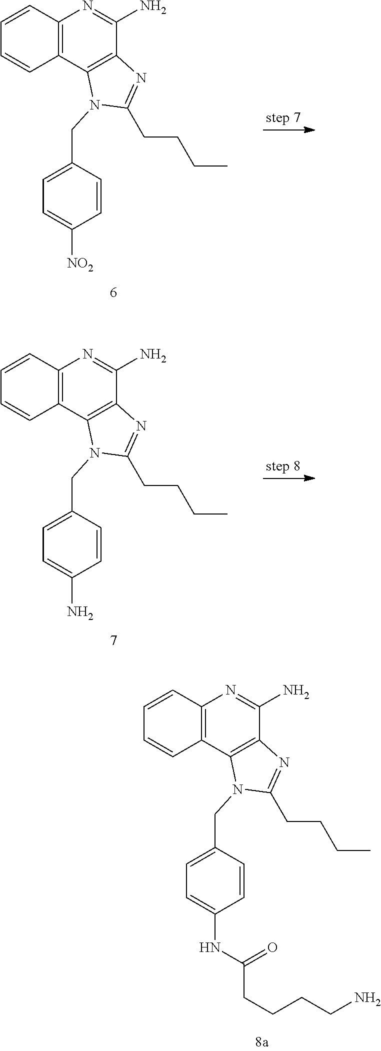 [SMILES: CCCCc1nc2c(N)nc3ccccc3c2n1Cc1ccc([N+](=O)[O-])cc1 ; CCCCc1nc2c(N)nc3ccccc3c2n1Cc1ccc(N)cc1 ; CCCCc1nc2c(N)nc3ccccc3c2n1Cc1ccc(NC(=O)CCCCN)cc1]

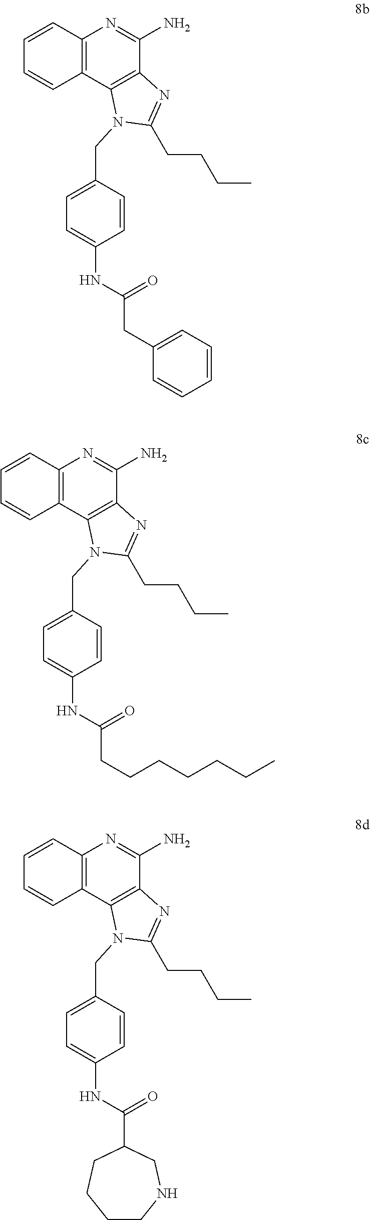 [SMILES: CCCCc1nc2c(N)nc3ccccc3c2n1Cc1ccc(NC(=O)Cc2ccccc2)cc1 ; CCCCCCCC(=O)Nc1ccc(Cn2c(CCCC)nc3c(N)nc4ccccc4c32)cc1 ; CCCCc1nc2c(N)nc3ccccc3c2n1Cc1ccc(NC(=O)C2CCCCNC2)cc1]

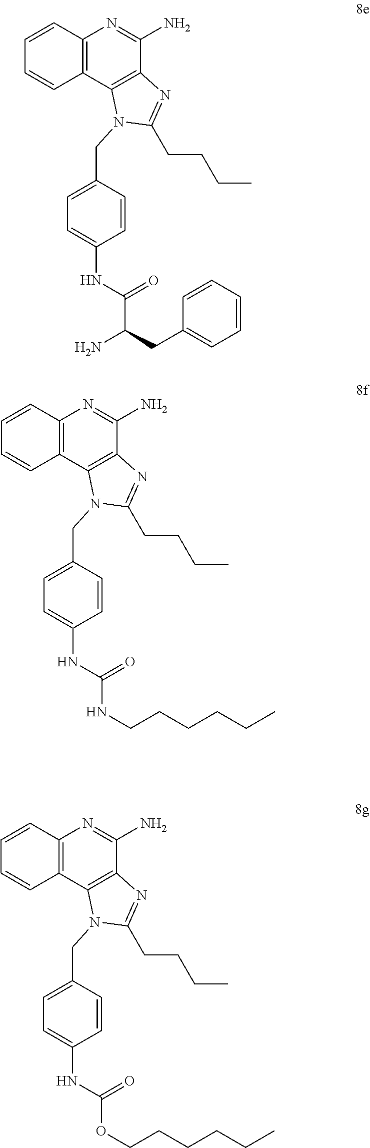 [SMILES: CCCCc1nc2c(N)nc3ccccc3c2n1Cc1ccc(NC(=O)[C@H](N)Cc2ccccc2)cc1 ; CCCCCCNC(=O)Nc1ccc(Cn2c(CCCC)nc3c(N)nc4ccccc4c32)cc1 ; CCCCCCOC(=O)Nc1ccc(Cn2c(CCCC)nc3c(N)nc4ccccc4c32)cc1]

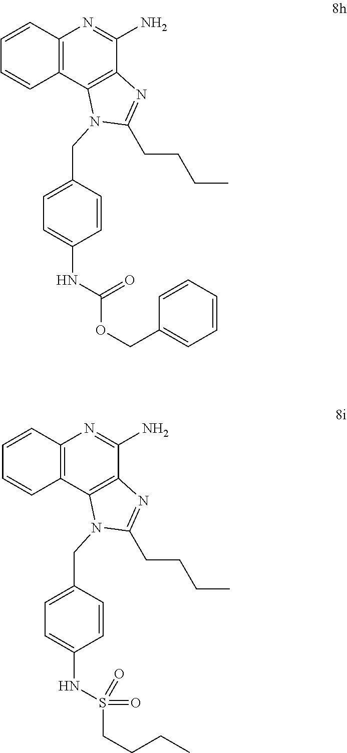 [SMILES: CCCCc1nc2c(N)nc3ccccc3c2n1Cc1ccc(NC(=O)OCc2ccccc2)cc1 ; CCCCc1nc2c(N)nc3ccccc3c2n1Cc1ccc(NS(=O)(=O)CCCC)cc1]

Step 1: 4-Chloro-3-nitroquinoline is treated with ammonia in dioxane at an elevated temperature (100-120° C.) to result in the formation of compound 1. Step 2-3: Palladium catalyzed hydrogenation of this compound in ethanol results in the formation of compound 2, which is, in turn, refluxed in caproic acid to result in the formation of the 2-alkyl-1H-imidazo[4,5-c]quinoline 3. Step 4: Selective alkylation of this compound with 4-nitrobenzyl bromide in DMF results in the formation of compound 4. This reaction is promoted by cesium carbonate or potassium carbonate and can be conducted at either room temperature or elevated temperature. The minor isomer of this transformation (alkylation at the other imidazole nitrogen) is easily separated from the desired product by silica gel chromatography. Step 5: Oxidation of the quinoline with meta-chloroperbenzoic acid (mCPBA) in chloroform results in the formation of the N-oxide 5. Step 6: Treatment of this intermediate with tosyl chloride and excess ammonia results in the formation of the aminoquinoline 6. Step 7: Treatment of compound 6 with iron and ammonium chloride at elevated temperature in ethanol results in the formation of key intermediate 7. Step 8: Boc-protected 5-aminocaproic acid is coupled with compound 7 using HATU and HOBt. Deprotection with TFA gives compound 8a. The intermediate 7 is similarly modified by acylation, sulfonylation, or carbamoylation to results in compounds 8b-8i. Final compounds are purified by silica gel chromatography or preparative HPLC.

An alternative preparation of a subset of various TLR agonists that are poised for attachment of the linker and antibody is shown in Scheme P2.

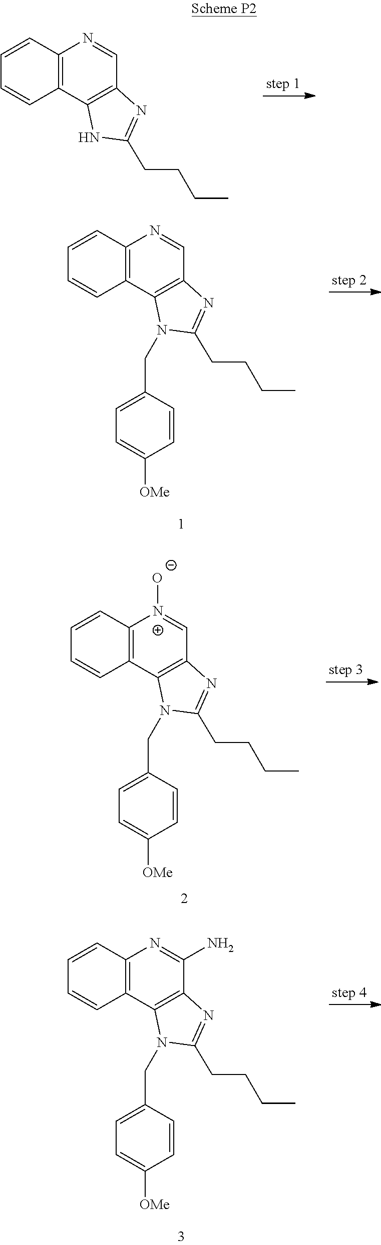 [SMILES: CCCCc1nc2cnc3ccccc3c2[nH]1 ; CCCCc1nc2cnc3ccccc3c2n1Cc1ccc(OC)cc1 ; CCCCc1nc2c[n+]([O-])c3ccccc3c2n1Cc1ccc(OC)cc1 ; CCCCc1nc2c(N)nc3ccccc3c2n1Cc1ccc(OC)cc1]

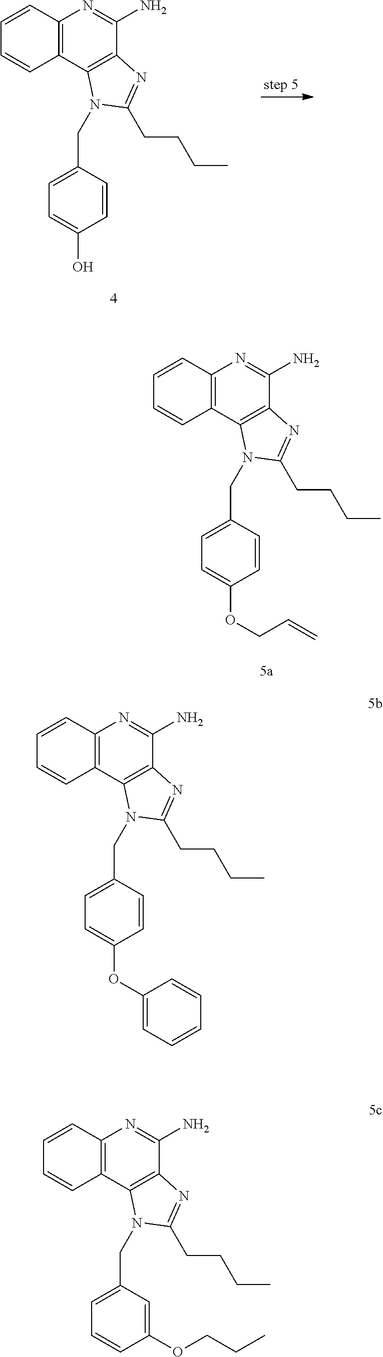 [SMILES: CCCCc1nc2c(N)nc3ccccc3c2n1Cc1ccc(O)cc1 ; C=CCOc1ccc(Cn2c(CCCC)nc3c(N)nc4ccccc4c32)cc1 ; CCCCc1nc2c(N)nc3ccccc3c2n1Cc1ccc(Oc2ccccc2)cc1 ; CCCCc1nc2c(N)nc3ccccc3c2n1Cc1cccc(OCCC)c1]

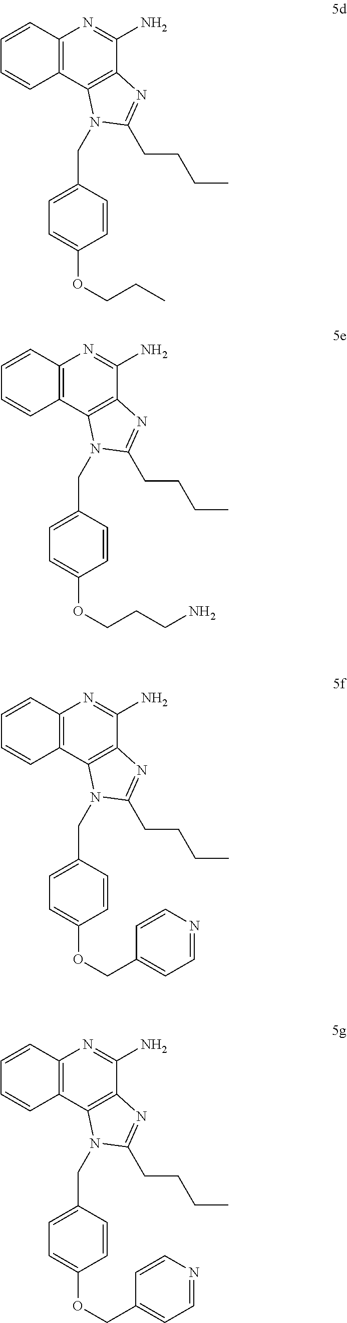 [SMILES: CCCCc1nc2c(N)nc3ccccc3c2n1Cc1ccc(OCCC)cc1 ; CCCCc1nc2c(N)nc3ccccc3c2n1Cc1ccc(OCCCN)cc1 ; CCCCc1nc2c(N)nc3ccccc3c2n1Cc1ccc(OCc2ccncc2)cc1 ; CCCCc1nc2c(N)nc3ccccc3c2n1Cc1ccc(OCc2ccncc2)cc1]

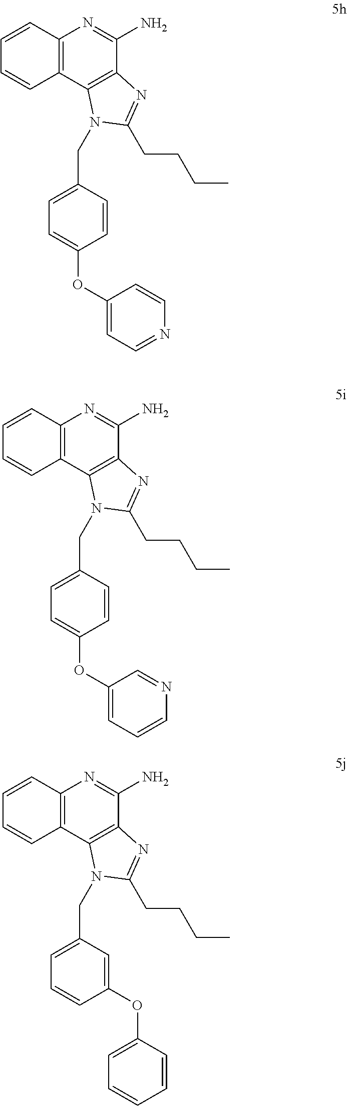

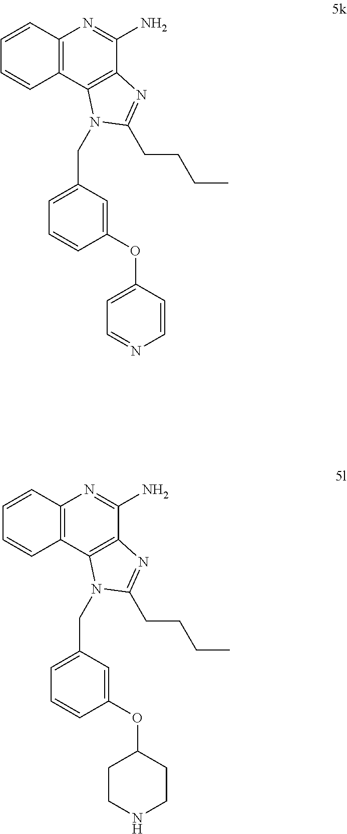 [SMILES: CCCCc1nc2c(N)nc3ccccc3c2n1Cc1cccc(Oc2ccncc2)c1 ; CCCCc1nc2c(N)nc3ccccc3c2n1Cc1cccc(OC2CCNCC2)c1]

The starting point for this reaction sequence is 2-butyl-1H-imidazo[4,5-c]quinoline, prepared as described above. This compound is selectively alkylated with 4-methoxybenzyl bromide in DMF at room temperature to give compound 1. This reaction is promoted by cesium carbonate or potassium carbonate and can be conducted at either room temperature or elevated temperature. The minor isomer of this transformation (alkylation at the other imidazole nitrogen) is easily separated from the desired product by silica gel chromatography. Step 2: Oxidation of the quinoline with meta-chloroperbenzoic acid (mCPBA) in chloroform results in the formation of the N-oxide 2. Step 3: Treatment of this intermediate with tosyl chloride and excess ammonia results in the formation of the aminoquinoline 3. Step 4: Demethylation using excess trimethylsilyl iodide in chloroform results in the formation of key phenol 4. Alkylation of the phenol with allyl chloride in DMF and cesium carbonate results in the formation 5a. Likewise, arylation of the phenol following the conditions of Cheng (Tetrahedron Letters, 53, 1, p 71-75) results in the formation of 5b. In short, the intermediate 4 is treated with iodobenzene (1.5 eq), Cu₂O (1 mol %), 1H-imidazole-4carboxylic acid (2 mol %), and cesium carbonate (2 eq) in acetonitrile at 80° C. The desired product (5b) is purified by silica gel chromatography using an EtOAc/Hex gradient.

### Example 12. Preparation of ADCs

The linker-payloads described in Examples 5-11 were conjugated with various antibodies resulting in the ADCs shown in Table 1. The conjugation was accomplished using the methods described below:

Method A: 2 mg of antibody in PBS was treated with 12 equivalents of 5 mM tris(2-carboxyethyl)phosphine (TCEP) for a final protein concentration of ˜5 mg/mL. The reaction was heated at 37° C. for an hour. 25 equivalents of linker-payload in DMA was added to the reaction along with sufficient DMA and PBS to result in a final organic of ˜5% (vol/vol) and final antibody concentration of 26.7 uM (4 mg/mL). The reaction sat at room temperature for 90 minutes. The reaction was then buffer exchanged into 100% PBS using a Sephadex column according to the manufacture's protocol. A small aliquot was reduced using TCEP and tested for its loading using HPLC-MS and the drug to antibody ratio (DAR) was calculated based on relative peak heights. The concentration of ADC was found using the Nanodrop using the Protein A280 IgG method and aggregation of an unreduced aliquot was analyzed using Size-Exclusion Chromatography. The final ADC was filter sterilized prior to storage.

For ADC's that exhibited a DAR under 6, the crude ADC was spun down using a 30kd centrifuge spin device to concentrate the sample and the ADC was resubmitted to TCEP and linker-payload treatment in the same order as stated above.

Method B: 2 mg of antibody was treated with 12 equivalents of 5 mM tris(2-carboxyethyl)phosphine (TCEP) and 0.1 M PBS pH 7.4 was added to the reaction to have a final antibody concentration of 26.7 uM (4 mg/mL) and was heated at 37° C. for two hours. The reaction was buffer exchanged using a Sephadex column according to the manufacture's protocol and concentrated using a centrifuge spin device with a 30K filter. 25 equivalents of linker-payload in DMA was added to the reaction along with sufficient DMA and PBS to result in a final organic of ˜5% (vol/vol) and final antibody concentration of 26.7 uM (4 mg/mL). The reaction sat at room temperature for 90 minutes. The reaction was then buffer exchanged into 100% PBS using a Sephadex column according to the manufacture's protocol. A small aliquot was reduced using TCEP and tested for its loading using HPLC-MS and the drug to antibody ratio (DAR) was calculated based on relative peak heights. The concentration of ADC was found using the Nanodrop using the Protein A280 IgG method and aggregation of an unreduced aliquot was analyzed using Size-Exclusion Chromatography. The final ADC was filter sterilized prior to storage.

For ADC's that exhibited a DAR under 6, the crude ADC was spun down using a 30kd centrifuge spin device to concentrate the sample and the ADC was resubmitted to TCEP and linker-payload treatment in the same order as stated above.

Method C: 2 mg of antibody was treated with 12 equivalents of 5 mM tris(2-carboxyethyl)phosphine (TCEP) and 0.1 M PBS pH 7.4 with 5 mM EDTA was added to the reaction to have a final antibody concentration of 26.7 uM and was heated at 37° C. for two hours. The reaction was buffer exchanged using a Sephadex column according to the manufacture's protocol and concentrated using a centrifuge spin device with a 30K filter. 25 equivalents of linker-payload in DMA was added to the concentrated reduced antibody along with 0.1 M PBS pH 7.4 with 5 mM EDTA containing 5% DMA (v/v) to result in a final antibody concentration of 26.7 uM (4 mg/mL). The reaction sat at room temperature for an hour and a half. Then, the reaction was buffer exchanged using a Sephadex column according to the manufacture's protocol. An aliquot was reduced using TCEP and tested for its loading using HPLC-MS and the drug to antibody ratio (DAR) was calculated. The concentration of ADC was found using the Nanodrop using the Protein A280 IgG method and aggregation of an unreduced aliquot was analyzed using size-exclusion chromatography on the UPLC. The final ADC was purified via filter sterilization.

For ADC's that exhibited a DAR under 6, the crude ADC was spun down using a 30kd centrifuge spin device to concentrate the sample and the ADC was resubmitted to TCEP and linker-payload treatment in the same order as stated above.

TABLE 1
List of ADCs.

| | | | | Actual | | | |
|---|---|---|---|---|---|---|---|
| | | | | Mass | Theoretical | | |
| | | | | shift | Mass shift | Aggregation | |
| ADC# | LP name | Antibody | DAR | (Da) | (Da) | (%) | Method |
| ADC#1 | LP#4 | Anti-Her2 (deglycosyl) | 6.5 | 914 | 913 | <5% | C |
| ADC#2 | LP#4 | Anti-CD20 (deglycosyl) | 6.8 | 910 | 913 | <5% | C |
| ADC#3 | LP#1 | Anti-Her2 (LLQG) | 4.1 | 690 | 688 | 25% | A |
| ADC#4 | LP#3 | Anti-Her2 (LLQG) | 7.7 | 762 | 762 | <5% | A |
| ADC#5 | LP#4 | Anti-Her2 (LLQG) | 7.3 | 914 | 913 | <5% | A |
| ADC#6 | LP#1 | Anti-CD20- | 5.2 | 690 | 688 | <5% | A |
| ADC#7 | LP#3 | Anti-CD20- | 7.7 | 764 | 762 | <5% | A |
| ADC#8 | LP#4 | Anti-CD20- | 7.3 | 508 | 913 | <5% | A |
| ADC#9 | LP#3 | Anti-Her2 (LLQG) | 6 | 764 | 762 | <5% | A |
| ADC#10 | LP#4 | Anti-Her2 (LLQG) | 6.9 | 914 | 913 | <5% | A |
| ADC#11 | LP#3 | Anti-CD20 (deglycosyl) | 6.9 | 764 | 762 | <5% | A |
| ADC#12 | LP#4 | Anti-CD20 (deglycosyl) | 7.4 | 914 | 913 | <5% | A |
| ADC#13 | LP#6 | Anti-Her2 (LLQG) | 8 | 960 | 958 | <5% | B |
| ADC#14 | LP#1 | Anti-Her2 (LLQG) | 7.8 | 694 | 688 | <5% | B |
| ADC#15 | LP#9 | Anti-Her2 (LLQG) | 4.8 | 952 | 944 | <5% | B |
| ADC#16 | LP#1 | Anti-CD20 (deglycosyl) | 8 | 690 | 688 | <5% | B |
| ADC#17 | LP#6 | Anti-CD20 (deglycosyl) | 8 | 958 | 958 | <5% | B |
| ADC#18 | LP#9 | Anti-CD20 (deglycosyl) | 8 | 950 | 944 | 9% | B |
| ADC#19 | LP#12 | Anti-Her2 (LLQG) | 7.7 | 916 | 912 | <5% | B |
| ADC#20 | LP#12 | Anti-CD20 (deglycosyl) | 7.6 | 914 | 912 | <5% | B |
| ADC#21 | LP#5 | Anti-Her2 (LLQG) | 7.8 | 510 | 507 | <5% | B |
| ADC#22 | LP#5 | Anti-CD20 (deglycosyl) | 8 | 508 | 507 | <5% | B |
| ADC#23 | LP#5 | Anti-Her2 (LLQG) | 6.9 | 432 | 432 | <5% | B |
| ADC#24 | LP#5 | Anti-CD20 (deglycosyl) | 7.6 | 432 | 432 | <5% | B |
| ADC#25 | LP#13 | Anti-Her2 (LLQG) | 8 | 510 | 506 | <5% | B |
| ADC#26 | LP#13 | Anti-CD20 (deglycosyl) | 8 | 506 | 506 | <5% | B |
| ADC#27 | LP#15 | Anti-CD20 (deglycosyl) | 5.3 | 522 | 526 | <5% | B |
| ADC#28 | LP#17 | Anti-Her2 (LLQG) | 4.7 | 512 | 512 | 16% | B |
| ADC#29 | LP#17 | Anti-CD20 (deglycosyl) | 6.6 | 512 | 512 | <5% | B |
| ADC#30 | LP#15 | Anti-Her2 (LLQG) | 2 | 564 | 526 | <5% | B |
| ADC#31 | LP#9 | Tocilizumab | 8 | 942 | 944 | <5% | B |
| ADC#32 | LP#9 | Ramucirumab | 8 | 946 | 944 | <5% | B |
| ADC#33 | LP#3 | Daratumumab | 8 | 762 | 762 | <5% | B |
| ADC#34 | LP#4 | Daratumumab | 8 | 909 | 913 | <5% | B |
| ADC#35 | LP#6 | Daratumumab | 8 | 958 | 958 | <5% | B |
| ADC#36 | LP#3 | Tocilizumab | 8 | 762 | 762 | <5% | B |
| ADC#37 | LP#4 | Tocilizumab | 8 | 910 | 913 | <5% | B |
| ADC#38 | LP#6 | Tocilizumab | 8 | 957 | 958 | <5% | B |
| ADC#39 | LP#3 | Ramucirumab | 8 | 761 | 762 | <5% | B |
| ADC#40 | LP#4 | Ramucirumab | 8 | 910 | 913 | <5% | B |
| ADC#41 | LP#6 | Ramucirumab | 7.2 | 960 | 958 | <5% | B |
| ADC#42 | LP#9 | Daratumumab | 2.9 | 945 | 944 | <5% | B |
| ADC#43 | LP#9 | Tocilizumab | 3.3 | 942 | 944 | <5% | B |
| ADC#44 | LP#9 | Ramucirumab | 2.5 | 946 | 944 | <5% | B |
| ADC#45 | LP#3 | Anti-Her2 (LLQG) | 8 | 764 | 762 | <5% | B |
| ADC#46 | LP#6 | Anti-CD20 (deglycosyl) | 6 | 958 | 958 | <5% | B |
| ADC#47 | LP#4 | Daratumumab | 8 | 906 | 913 | <5% | B |
| ADC#48 | LP#9 | Daratumumab | 8 | 946 | 944 | <5% | B |
| ADC#49 | LP#9 | Tocilizumab | 8 | 938 | 944 | <5% | B |
| ADC#50 | LP#10 | Anti-Her2 (LLQG) | 7.3 | 794 | 794 | <5% | B |
| ADC#51 | LP#10 | Anti-CD20 (deglycosyl) | 8 | 794 | 794 | <5% | B |
| ADC#52 | LP#11 | Anti-Her2 (LLQG) | 6 | 538 | 538 | <5% | B |
| ADC#53 | LP#11 | Anti-CD20 (deglycosyl) | 8 | 537 | 538 | <5% | B |
| ADC#54 | LP#7 | Anti-Her2 (LLQG) | 5.7 | 550 | 552 | <5% | B |
| ADC#55 | LP#7 | Anti-CD20 (deglycosyl) | 6 | 556 | 552 | <5% | B |
| ADC#56 | LP#5 | Daratumumab | 6 | 510 | 507 | <5% | C |
| ADC#57 | LP#7 | Daratumumab | 6 | 550 | 552 | <5% | C |
| ADC#58 | LP#8 | Daratumumab | 6.6 | 819 | 538 | <5% | C |
| ADC#59 | LP#11 | Daratumumab | 8 | 538 | 538 | <5% | C |
| ADC#60 | LP#10 | Daratumumab | 7 | 794 | 794 | <5% | C |
| ADC#61 | LP#8 | Anti-CD20 (deglycosyl) | 6.6 | 810 | 538 | <5% | C |
| ADC#62 | LP#6 | Anti-CD20 (deglycosyl) | 8 | 960 | 958 | <5% | C |
| ADC#63 | LP#11 | Polatuzumab | 5.2 | 537 | 538 | <5% | C |
| ADC#64 | LP#10 | Polatuzumab | 8 | 795 | 794 | <5% | C |
| ADC#65 | LP#9 | Polatuzumab | 8 | 942 | 944 | <5% | C |
| ADC#66 | LP#6 | Polatuzumab | 8 | 960 | 958 | <5% | C |
| ADC#67 | LP#4 | Polatuzumab | 8 | 913 | 913 | <5% | C |
| ADC#68 | LP#9 | Anti-Her2 (LLQG) | 5.5 | 944 | 944 | 20% | C |
| ADC#69 | LP#8 | Anti-Her2 (LLQG) | 6.3 | 810 | 538 | <5% | C |

### Example 13: Evaluation of Payloads Against Ramos-Blue Cells Using a 24 h or 72 h Assay

A 5× serial dilution was performed in 10% DMSO in PBS for each payload to have a final range of concentration from 1000 uM to 64 nM. Ramos-blue cells (InvivoGen, cat #rms-sp) were cultured using high glucose DMEM media supplied with 10% fetal bovine serum according to the manufacturer guidelines. The media was supplemented with 50 U/mL penicillin, 50 ug/mL streptomycin, and 100 ug/mL normocin to prevent bacterial contamination. The cell density and viability were calculated using a Countess Cell Counter and the proper volume of cells was removed in order to have a seeding density of 0.2×10⁶ cells/mL per well. 135 uL of the cell suspension was added to each well in a 96-well plate along with 15 uL of the corresponding payload treatment. Each assay point was run in triplicate and the plate was incubated at 37° C. with 5% CO₂ for 24 or 72 hours.

In order to assess the NFκB induction, the QUANTI-Blue™ solution was prepared by adding 200 uL of QB reagent (InVivogen cat #rep-qbs) and 200 uL of QB buffer to 19.6 mL of water. The resulting solution was vortexed and incubated at room temperature for ten minutes. The 96-well plate was centrifuged at 1990 rpm for ten minutes and 40 uL of the cell supernatant was added to 160 uL of the prepared QUANTI-Blue™ solution. The QB reaction plate was incubated at 37° C. for 24 hours. The plate was read using the Molecular Devices i3x plate reader at a wavelength of 630 nm to determine the amount of SEAP production.

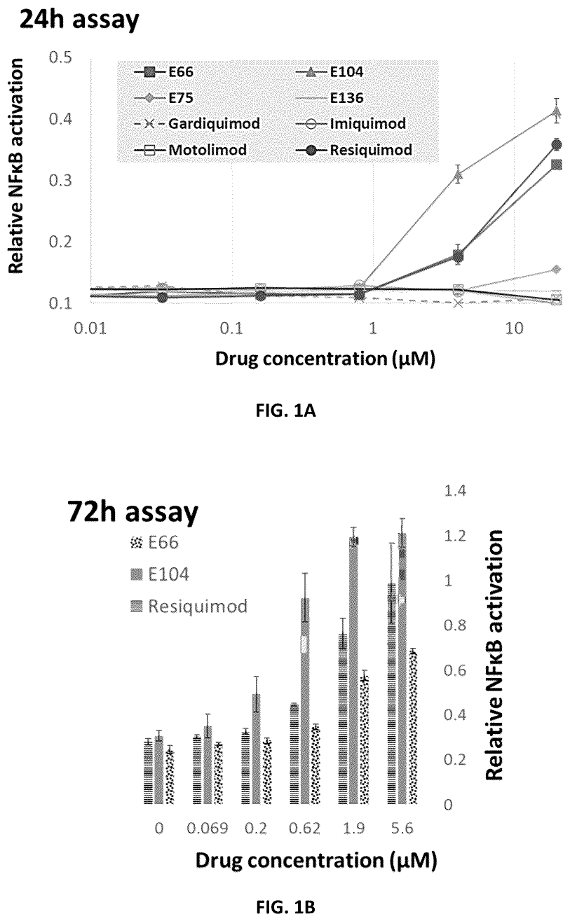

The data shown in FIG. 1A and FIG. 1B demonstrate that select payloads of the invention are able to activate the NFκB signaling pathway in a lymphocyte cell line that expresses both TLR7 and TLR8.

### Example 14. Evaluation of Payloads Against a mTLR7-Expressing HEK293-Reporter Cell Line

The mouse TLR7 expressing HEK-Blue™ mTLR7 cell line was purchased from InvivoGen. (cat #hkb-mtlr7) The HEK-Blue™ mTLR7 cells were maintained in culture media using high glucose DMEM media supplied with 10% fetal bovine serum. The media was supplemented with 50 U/mL penicillin, 50 ug/mL streptomycin, and 100 ug/mL normocin to prevent bacterial contamination. Before the experiment, HEK-Blue™ mTLR7 cells were rinsed and detached using prewarmed DPBS. The cells were collected and centrifuged at 1100 rpm for 5 minutes to remove supernatant. The cells were then re-suspended into a 0.2 million cells per mL seeding suspension and seeded to 96 well plates with a seeding volume of 90 uL. Resiquimod, E66, and E104 were diluted to 30, 3, 0.3, and 0.03 uM. 10 uL of the payload solutions were added to corresponding wells to reach a final concentration gradient of 3, 0.3, 0.03, and 0.003 uM. 10 uL DPBS was added to for the blank.

The experiment was performed in triplicate. The plate was incubated under a 37 degrees Celsius/5% CO₂ environment for 24 hours. The supernatants were collected, and NFκB activation was detected by running a QUANTI-Blue™ assay. Briefly, the QUANTI-Blue™ reagent purchased from InvivoGen was reconstituted into a detection solution following the manufacturer's protocol. 180 uL of QUANTI-Blue™ detection solution was mixed with 20 uL of supernatant and incubated in a 37 degrees Celsius/5% CO₂ environment for 4 hours. The absorbance at 630 nm was then detected using a SpectraMax i3X microplate reader. The data was analyzed and plotted using GraphPad Prism 7 software.

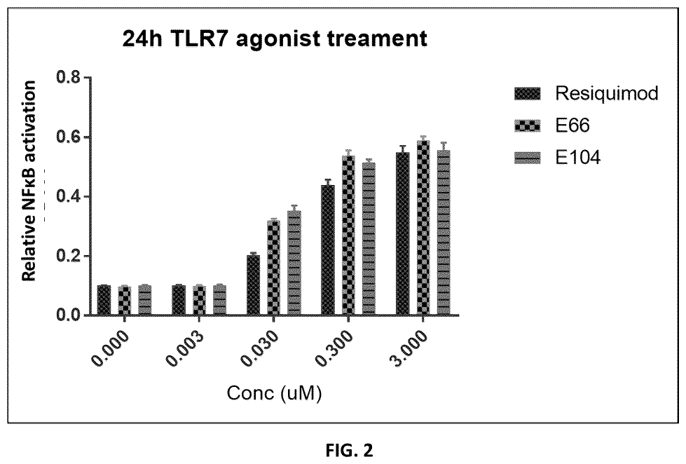

The data shown in FIG. 2 demonstrates that payloads disclosed herein agonize the mouse-TLR7 pathway.

### Example 15. Attachment of Linkers Abolishes TLR Agonist Activity in Ramos Blue Reporter Assay

A 50 mM cysteine stock was prepared in 0.1 M PBS pH 7.4. 10 equivalents of 50 mM cysteine were added to 1 equivalent of linker-payload to create a 2.50 mM solution. The reaction was vortexed and sat at room temperature for an hour to allow for the Michael addition to occur. After an hour, an aliquot was taken and run via UPLC-MS to ensure for the complete Michael reaction. The reaction was diluted to 1 mM using 0.1M PBS pH 7.4. The 1 mM solution was diluted to 500 uM and 50 uM in a 96-well plate to afford a final concentration of 50 uM and 5 uM in the cell suspension. The cell density and viability were calculated using the Countess Cell Counter and the proper volume of cells was removed in order to have a seeding density of 0.2×10⁶ cells/mL per well. 135 uL of cell suspension was added to each well in a 96-well plate along with 15 uL of the corresponding linker-payload treatment. Each concentration was run in triplicate and incubated at 37° C. with 5% CO₂ for 72 hours.

Invivogen's QUANTI-Blue™ solution was prepared by adding 200 uL of QB reagent and 200 uL of QB buffer to 19.6 mL of water. The resulting solution was vortexed and incubated at room temperature for ten minutes. The 96-well plate was centrifuged at 1990 rpm for ten minutes. Then, 40 uL of cell supernatant was added to 160 uL of the prepared QUANTI-Blue™ solution and incubated at 37° C. for 24 hours. The plate was read using the Molecular Devices i3x plate reader at a wavelength of 630 nm to determine the amount of SEAP production.

The data shown in Table 2 illustrates the propensity of the linker-payloads to induce NFκB activation.

TABLE 2
Table showing activation of NFκB pathway in Ramos-
blue cells by linker-payloads of the invention.

| | Linker-Payload | Relative | |
|---|---|---|---|
| Linker-payload | Dose (uM) | Activation | STD |
| Mc_E104 | 5 | 704.00% | 202.09% |
| Mc_E104 | 50 | −51.98% | 14.47% |
| Mc_ValCit_E104 | 5 | 237.56% | 319.56% |
| Mc_ValCit_E104 | 50 | −50.90% | 10.17% |
| Mc_ValCitPABC_E104 | 5 | 12.73% | 212.04% |
| Mc_ValCitPABC_E104 | 50 | −19.22% | 50.34% |
| Mc_E66 | 5 | 118.60% | 415.42% |
| Mc_E66 | 50 | −52.32% | 34.48% |
| Mc_ValCit_E66 | 5 | 124.81% | 419.20% |
| Mc_ValCit_E66 | 50 | −51.37% | 28.33% |
| Mc_ValCitPABC_E66 | 5 | 7.15% | 154.20% |
| Mc_ValCitPABC_E66 | 50 | −19.66% | 115.31% |
| Mc_Resiquimod | 5 | 17.58% | 259.14% |
| Mc_Resiquimod | 50 | −20.98% | 22.45% |
| Mc_ValCit_Resiquimod | 5 | 11.41% | 148.92% |
| Mc_ValCit_Resiquimod | 50 | −23.13% | 37.13% |
| Mc_ValCitPABC_Resiquimod | 5 | 18.62% | 233.29% |
| Mc_ValCitPABC_Resiquimod | 50 | −20.76% | 38.92% |

### Example 16. Activation of NFκB Activity in Ramos-Blue Cells by TLR-Agonist ADCs

Ramos blue assays with ADCs were optimized using a seeding density of 0.5×10⁶ cells/mL and a 72-hour incubation time to a 1×10⁶ cells/mL and 96-hour incubation.

ADCs were diluted into to a concentration of 1000 μg/mL and 300 μg/mL in PBS. The cell density and viability were calculated using the Countess Cell Counter and the proper volume of cells was removed in order to have a seeding density of 1×10⁶ cells/mL per well. 80 uL of cell suspension was added to each well in a 96-well plate along with 20 uL of the corresponding ADC treatment. Final ADCs concentrations of 100 ug/mL and 30 ug/mL were evaluated. Each assay condition was performed in quadruplicate and incubated at 37° C. with 5% CO₂ for 96-hours.

In select examples, cells were treated with a pre-dose of 100 ug/mL naked antibody and were incubated for 15 minutes at 37° C. with 5% CO₂ prior to ADC treatment. Cells were treated with 10 uL of naked antibody treatment followed by 10 uL of ADC treatment after the 15-minute incubation. Samples were run in quadruplicate and incubated for 96-hours.

Invivogen's QUANTI-Blue™ solution was prepared by adding 200 uL of QB reagent and 200 uL of QB buffer to 19.6 mL of water. The resulting solution was vortexed and incubated at room temperature for ten minutes. The 96-well plate was centrifuged at 1990 rpm for ten minutes. Then, 40 uL of cell supernatant was added to 160 uL of the prepared QUANTI-Blue™ solution and incubated at 37° C. for 24 hours. The plate was read using the Molecular Devices i3x plate reader at a wavelength of 630 nm to determine the amount of SEAP production.

The data shown in Table 3 illustrates that ADCs of the present invention activate the NFκB pathway in a B-cell reporter assay, thus showing potential as selective immune-modulating agents.

TABLE 3

| | | | Co-dose | ADC | | | | |
|---|---|---|---|---|---|---|---|---|
| | | | (100 | Dose | Absolute | | Relative | |
| ADC# | Antibody | Linker payload | ug/mL) | (ug/mL) | activation | STD | activation | STD |
| ADC#59 | Anti-CD38 | None | | 100 | 0.608 | 0.2183 | 369.63% | 132.76% |
| ADC#56 | Anti-CD38 | None | | 100 | 0.231 | 0.0877 | 140.52% | 53.32% |
| ADC#58 | Anti-CD38 | mc_ValCitPABC_E104 | | 100 | 0.230 | 0.0719 | 139.67% | 43.70% |
| ADC#2 | Anti-CD20 | mc_ValCitPABC_Resiquimod | | 30 | 0.167 | 0.0258 | 62.09% | 9.59% |
| | (Degly) | | | | | | | |
| | Anti-CD20 | mc_ValCitPABC_Resiquimod | | 100 | 0.197 | 0.0470 | 73.24% | 17.44% |
| | (Degly) | | | | | | | |
| ADC#2 | Anti-CD20 | mc_ValCitPABC_E66 | | 100 | 0.197 | 0.0262 | 73.11% | 9.72% |
| | (Degly) | | | | | | | |
| | Anti-CD20 | mc_ValCitPABC_E104 | | 30 | 0.174 | 0.0134 | 64.43% | 4.98% |
| | (Degly) | | | | | | | |
| ADC#57 | Anti-CD38 | mc_ValCitPABC_E66 | | 100 | 0.107 | 0.0951 | 65.33% | 57.83% |
| ADC#18 | Anti-CD20 | mc_E104 | | 30 | 0.165 | 0.0380 | 61.18% | 14.11% |
| | (Degly) | | | | | | | |
| ADC#18 | Anti-CD20 | mc_Resiquimod | | 100 | 0.152 | 0.0219 | 56.34% | 8.14% |
| | (Degly) | | | | | | | |
| ADC#17 | Anti-CD20 | mc_E104 | | 100 | 0.146 | 0.0148 | 54.26% | 5.51% |
| | (Degly) | | | | | | | |
| ADC#59 | Anti-CD38 | mc_Resiquimod | | 30 | 0.089 | 0.0631 | 54.17% | 38.36% |
| ADC#17 | Anti-CD20 | mc_ValCit_E66 | | 30 | 0.137 | 0.0251 | 51.01% | 9.33% |
| | (Degly) | | | | | | | |
| ADC#53 | Anti-CD20 | mc_E66 | | 30 | 0.096 | 0.0153 | 50.87% | 8.09% |
| | (Degly) | | | | | | | |
| ADC#33 | Anti-CD38 | mc_E104 | | 100 | 0.083 | 0.0171 | 50.32% | 10.37% |
| ADC#34 | Anti-CD38 | mc_Resiquimod | | 100 | 0.077 | 0.0308 | 47.08% | 18.76% |
| ADC#34 | Anti-CD38 | mc_ValCit_E66 | | 100 | 0.105 | 0.0184 | 39.04% | 6.82% |
| ADC#35 | Anti-CD38 | mc_ValCitPABC_Resiquimod | | 100 | 0.059 | 0.0240 | 35.62% | 14.59% |
| ADC#33 | Anti-CD38 | None | | 30 | 0.056 | 0.0038 | 33.97% | 2.31% |
| ADC#42 | Anti-CD38 | mc_ValCitPABC_Resiquimod | | 100 | 0.054 | 0.0233 | 32.75% | 14.19% |
| ADC#34 | Anti-CD38 | None | | 30 | 0.046 | 0.0159 | 17.26% | 5.91% |
| ADC#35 | Anti-CD38 | mc_E66 | | 30 | 0.043 | 0.0102 | 26.08% | 6.19% |
| ADC#42 | Anti-CD38 | mc_ValCitPABC_E104 | | 30 | 0.033 | 0.0139 | 20.09% | 8.44% |
| ADC#50 | Anti-Her2 | mc_ValCitPABC_E104 | | 30 | 0.035 | 0.0126 | 18.44% | 6.67% |
| ADC#34 | Anti-CD38 | mc_ValCitPABC_E66 | | 30 | 0.023 | 0.0056 | 13.82% | 3.41% |
| ADC#63 | Anti-CD79 | mc_E104 | | 30 | 0.020 | 0.0202 | 10.45% | 10.68% |
| | Anti-CD79 | mc_ValCitPABC_E66 | | 30 | 0.020 | 0.0159 | 10.33% | 8.43% |
| ADC#65 | Anti-CD79 | mc_E104 | | 30 | 0.018 | 0.0170 | 9.51% | 9.02% |
| ADC#67 | Anti-CD79 | mc_ValCit_Resiquimod | | 30 | 0.008 | 0.0157 | 4.30% | 8.32% |
| ADC#35 | Anti-CD38 | mc_ValCitPABC_Resiquimod | | 30 | 0.017 | 0.0088 | 6.32% | 3.25% |
| | Anti-CD38 | mc_ValCitPABC_Resiquimod | | 100 | 0.020 | 0.0081 | 12.26% | 4.92% |
| ADC#66 | Anti-CD79 | mc_ValCitPABC_E66 | | 30 | 0.009 | 0.0216 | 4.71% | 11.42% |
| ADC#1 | Anti-Her2 | mc_ValCit_Resiquimod | | 100 | 0.019 | 0.0087 | 6.98% | 3.24% |
| | Anti-Her2 | mc_ValCitPABC_E104 | | 30 | 0.005 | 0.0148 | 2.74% | 7.85% |
| | Anti-CD38 | mc_ValCitPABC_Resiquimod | | 30 | 0.004 | 0.0068 | 1.36% | 2.52% |
| ADC#13 | Anti-Her2 | mc_ValCitPABC_E66 | | 100 | 0.010 | 0.0077 | 3.62% | 2.87% |
| ADC#13 | Anti-Her2 | mc_ValCitPABC_E104 | | 30 | 0.000 | 0.0094 | 0.01% | 3.51% |
| ADC#13 | Anti-Her2 | mc_ValCit_E104 | | 30 | −0.001 | 0.0135 | −0.29% | 7.14% |
| ADC#15 | Anti-Her2 | mc_ValCitPABC_Resiquimod | | 30 | −0.001 | 0.0182 | −0.69% | 9.63% |
| ADC#35 | Anti-CD38 | mc_E104 | | 100 | 0.003 | 0.0060 | 1.19% | 2.23% |
| | Anti-CD38 | None | | 100 | −0.004 | 0.0074 | −1.31% | 2.74% |
| | Anti-CD38 | mc_ValCitPABC_E104 | | 30 | −0.003 | 0.0072 | −1.65% | 4.35% |
| ADC#15 | Anti-Her2 | mc_ValCitPABC_Resiquimod | | 30 | −0.006 | 0.0101 | −2.40% | 3.74% |
| ADC#64 | Anti-CD79 | mc_ValCitPABC_E66 | | 30 | −0.005 | 0.0188 | −2.48% | 9.97% |
| ADC#15 | Anti-Her2 | None | | 100 | −0.007 | 0.0145 | −2.70% | 5.37% |
| ADC#58 | Anti-CD38 | mc_ValCitPABC_E66 | | 30 | −0.006 | 0.0126 | −3.76% | 7.64% |
| ADC#1 | Anti-Her2 | mc_ValCitPABC_Resiquimod | | 30 | 0.001 | 0.0051 | 0.44% | 1.88% |
| ADC#60 | Anti-CD38 | None | | 100 | −0.015 | 0.0160 | −8.98% | 9.71% |
| ADC#1 | Anti-Her2 | None | | 30 | −0.003 | 0.0117 | −1.52% | 6.19% |
| ADC#60 | Anti-CD38 | mc_ValCitPABC_E66 | | 30 | −0.028 | 0.0080 | −17.20% | 4.85% |
| ADC#56 | Anti-CD38 | mc_ValCitPABC_E66 | | 30 | −0.030 | 0.0064 | −17.95% | 3.87% |
| ADC#57 | Anti-CD38 | mc_ValCitPABC_E66 | | 30 | −0.042 | 0.0016 | −25.68% | 0.96% |
| ADC#34 | Anti-CD38 | mc_ValCitPABC_E104 | | 30 | 0.004 | 0.0122 | 2.05% | 6.86% |
| ADC#34 | Anti-CD38 | mc_ValCitPABC_E66 | | 100 | 0.174 | 0.0656 | 97.25% | 36.78% |
| ADC#1 | Anti-Her2 | None | | 30 | 0.009 | 0.0032 | 4.88% | 1.81% |
| ADC#1 | Anti-Her2 | None | | 100 | 0.030 | 0.0133 | 16.76% | 7.47% |
| ADC#34 | Anti-CD38 | mc_ValCitPABC_E104 | Anti- | 100 | −0.030 | 0.0072 | −16.56% | 4.06% |
| | | | CD38 | | | | | |
| | | | mAb | | | | | |
| ADC#34 | Anti-CD38 | mc_ValCit_E104 | Anti- | 100 | 0.176 | 0.0213 | 98.42% | 11.93% |
| | | | CD38 | | | | | |
| | | | mAb | | | | | |
| ADC#56 | Anti-CD38 | mc_ValCitPABC_E104 | | 30 | 0.108 | 0.0768 | 60.34% | 43.07% |
| ADC#56 | Anti-CD38 | mc_ValCit_E66 | | 100 | 0.476 | 0.0954 | 267.04% | 53.46% |
| ADC#21 | Anti-Her2 | mc_ValCitPABC_Resiquimod | | 30 | 0.089 | 0.0480 | 50.11% | 26.88% |
| ADC#21 | Anti-Her2 | mc_ValCit_E104 | | 30 | 0.884 | 0.3435 | 495.36% | 192.55% |
| ADC#56 | Anti-CD38 | mc_ValCitPABC_Resiquimod | Anti- | 30 | 0.036 | 0.0261 | 20.24% | 14.65% |
| | | | CD38 | | | | | |
| | | | mAb | | | | | |
| ADC#56 | Anti-CD38 | mc_ValCit_E104 | Anti- | 30 | 0.438 | 0.0632 | 245.45% | 35.43% |
| | | | CD38 | | | | | |
| | | | mAb | | | | | |
| ADC#42 | Anti-CD38 | mc_Resiquimod | | 100 | 0.003 | 0.0070 | 1.88% | 3.92% |
| ADC#42 | Anti-CD38 | mc_E66 | | 30 | 0.126 | 0.0556 | 70.73% | 31.17% |
| ADC#15 | Anti-Her2 | mc_ValCitPABC_Resiquimod | | 30 | 0.006 | 0.0073 | 3.57% | 4.09% |
| ADC#15 | Anti-Her2 | mc_ValCitPABC_Resiquimod | | 30 | 0.012 | 0.0029 | 6.54% | 1.64% |
| ADC#42 | Anti-CD38 | mc_ValCitPABC_Resiquimod | Anti- | 30 | −0.025 | 0.0103 | −13.93% | 5.76% |
| | | | CD38 | | | | | |
| | | | mAb | | | | | |
| ADC#42 | Anti-CD38 | mc_ValCitPABC_Resiquimod | Anti- | 30 | 0.072 | 0.0504 | 40.30% | 28.23% |
| | | | CD38 | | | | | |
| | | | mAb | | | | | |
| ADC#59 | Anti-CD38 | mc_ValCitPABC_Resiquimod | | 30 | 0.053 | 0.0294 | 29.79% | 16.49% |
| ADC#59 | Anti-CD38 | mc_ValCitPABC_Resiquimod | | 100 | 0.390 | 0.1627 | 218.44% | 91.19% |
| ADC#52 | Anti-Her2 | mc_Resiquimod | | 100 | −0.032 | 0.0075 | −17.67% | 4.22% |
| ADC#52 | Anti-Her2 | mc_Resiquimod | | 30 | 0.061 | 0.0576 | 33.95% | 32.26% |
| ADC#59 | Anti-CD38 | mc_Resiquimod | Anti- | 30 | 0.010 | 0.0223 | 5.70% | 12.52% |
| | | | CD38 | | | | | |
| | | | mAb | | | | | |
| ADC#59 | Anti-CD38 | mc_Resiquimod | Anti- | 100 | 0.290 | 0.0442 | 162.35% | 24.78% |
| | | | CD38 | | | | | |
| | | | mAb | | | | | |

### Example 17. Conditioned Media from Breast-Cancer Targeted ADCs are Able to Activate a mTLR7 Reporter System

The HER2 over-expressing breast-cancer cell line SKBR3 was maintained in high glucose DMEM media supplied with 10% fetal bovine serum. Before the experiment, SKBR3 cells were trypsinized, centrifuged, and re-suspended into a 0.2 million cells per mL seeding suspension. SKBR3 cells (100 uL, 20,000 cells) were then seeded to 96 well plates and allowed to adhere overnight. HEK-Blue™ mTLR7 cells were also seeded to 96 well plates as previously described with a density of 0.2 million per mL and a volume of 90 uL. Penicillin/streptomycin and Normocin™ were applied to prevent microbial contamination. TLR7 agonist ADCs were diluted to desired concentrations. 10 uL of ADC solution, ADC solution with naked monoclonal antibody, or DPBS were added to corresponding wells and mixed thoroughly. The experiment was performed in triplicate. After the treatment, SKBR3 cells were incubated in a 37° C./5% CO₂ environment for 48 hours. After the incubation, the supernatant of the HEK-Blue™ mTLR7 plates were replaced by the supernatants of corresponding wells of the SKBR3 plates. The HEK-Blue™ mTLR7 cells were incubated for 48 hours before the supernatants were collected and the NFκB level was measured by the QUANTI-Blue™ assay described in example 14. The data was analyzed and plotted using GraphPad Prism 7 software.

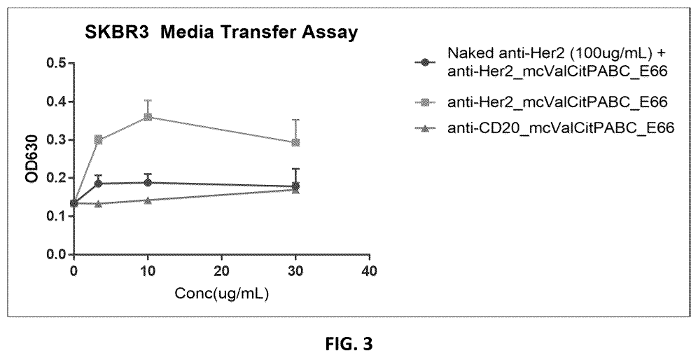

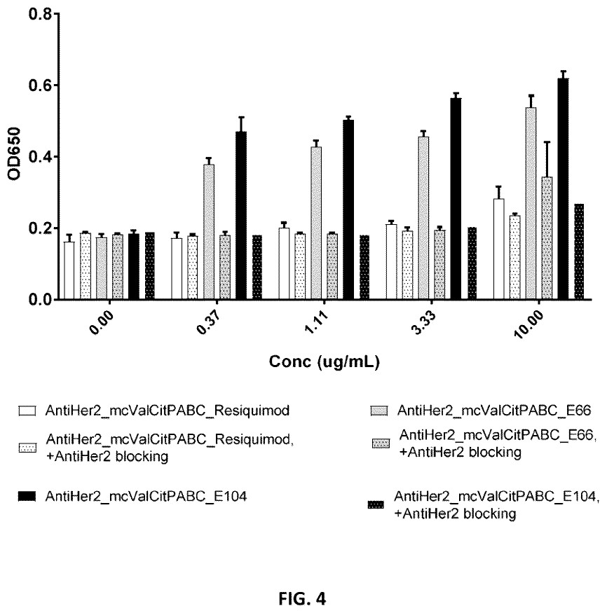

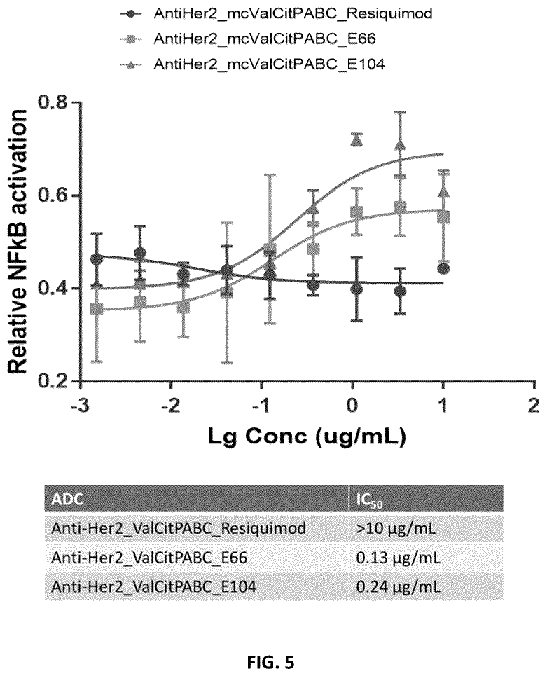

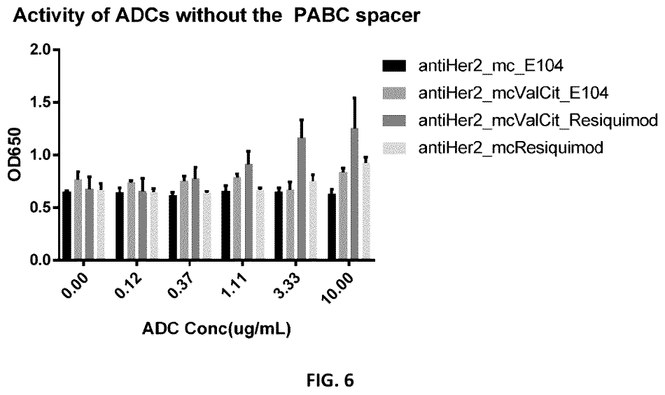

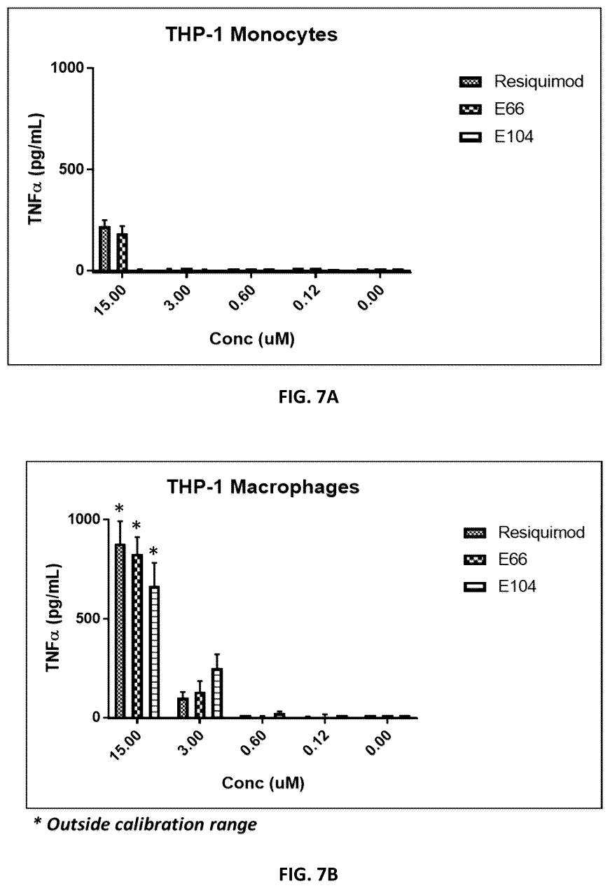

FIG. 3 shows that ADCs of interest in the invention can be metabolized by antigen-expressing tumor tissue to result in the activation of nearby TLR7-expressing cells. This activity can be suppressed by co-dosing of naked antibody or by targeting a non-expressed antigen. FIG. 4 and FIG. 5 show that E104 and E66 ADCs have superior activity as compared to resiquimod ADCs. FIG. 6 shows that alternative linkers (eliminating the PABC spacer) are not as efficient in activating nearby TLR7 cells.

### Example 18: Cytokine Release from Macrophages

The human monocyte line THP-1 was purchased from ATCC. Differentiation of the THP-1 into macrophages was accomplished as follows: THP-1 cells were seeded to 96 well plates (20,000 cells per well) and cultured in 100 uL of RPMI 1640 media supplied with 10% fetal bovine serum containing 200 nM phorbol-12-myristate-13-acetate (PMA) for 72 hours. After the incubation, the supernatant was removed, and the cells were rinsed with pre-warmed DPBS twice to eliminate remaining PMA. The cells were then cultured in fresh RPMI 1640 media supplied with 10% fetal bovine serum for at least 72 hours. Morphological evidence of macrophage differentiation was confirmed microscopically. To test the cytokine release from THP-1 and THP-1 differentiated macrophages, THP-1 monocytes were seeded to 96 well plates at a density of 0.2 million per mL with a volume of 90 uL. Old media from the macrophage plates were removed and 90 uL of fresh culture media was added. Stock solutions of the payloads were prepared at 150, 30, 6, and 1.2 uM. 10 uL of the payload solutions were added to the corresponding wells and mixed well to reach a final concentration gradient of 15, 3, 0.6, and 0.12 uM. The experiment was performed in triplicate, and penicillin/streptomycin was used to avoid bacterial contamination. 10 uL DPBS was added to the blank to keep volumes consistent. The cells were incubated in 37 degrees Celsius/5% CO₂ for 24 hours. After the incubation, the plates were centrifuged, and the supernatant samples were collected. TNF alpha levels in each supernatant sample were detected using a Duoset TNF alpha ELISA kit (R&D systems). The data was analyzed and plotted using GraphPad Prism 7 software.

FIG. 21 illustrates that compounds of the invention induce the release of TNFα from both macrophages and monocytes. The amount of cytokines released from macrophages is significantly higher than monocytes.

### Example 19: Evaluation of Serum Stability

100 ug/mL of ADC was added to 250 uL of mouse or human serum and brought up to a final volume of 400 uL using 0.1M PBS pH 7.4. A blank was prepared by adding 150 uL 0.1M PBS pH 7.4 to 250 uL of mouse or human serum. The reaction was incubated at 37° C. with 5% CO₂ for seven days. 50 uL aliquot was taken at time 0, 6 hours, and 1, 2, 4, and 7 days and frozen at −80° C. immediately to prevent further reaction.

Samples were defrosted and 20 uL was added to 40 uL ACN to induce protein precipitation. The sample was vortex and centrifuged at 100,000 rmp for 10 minutes. The 40 uL of supernatant was taken and added to 40 uL water. A standard curve was prepared via a 10× serial dilution in 1:3 ACN:Water to have a range from 10 uM to 0.1 nM. A 10 uL aliquot was injected into Waters Xevo TQD Mass Spectrometer using an optimized MRM method for quantification. Scanned transitions for Resiquimod are 314.40→152.02, 197.05, 251.09; transitions for E104 are 346.19→105.95, 197.96, 241.00; transitions for E66 are 360.25→92.25, 119.93, and 241.01.

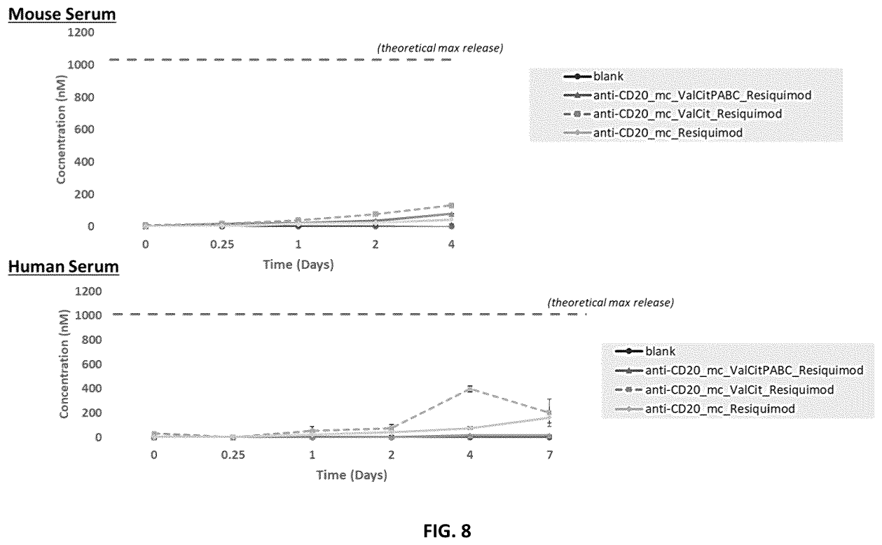

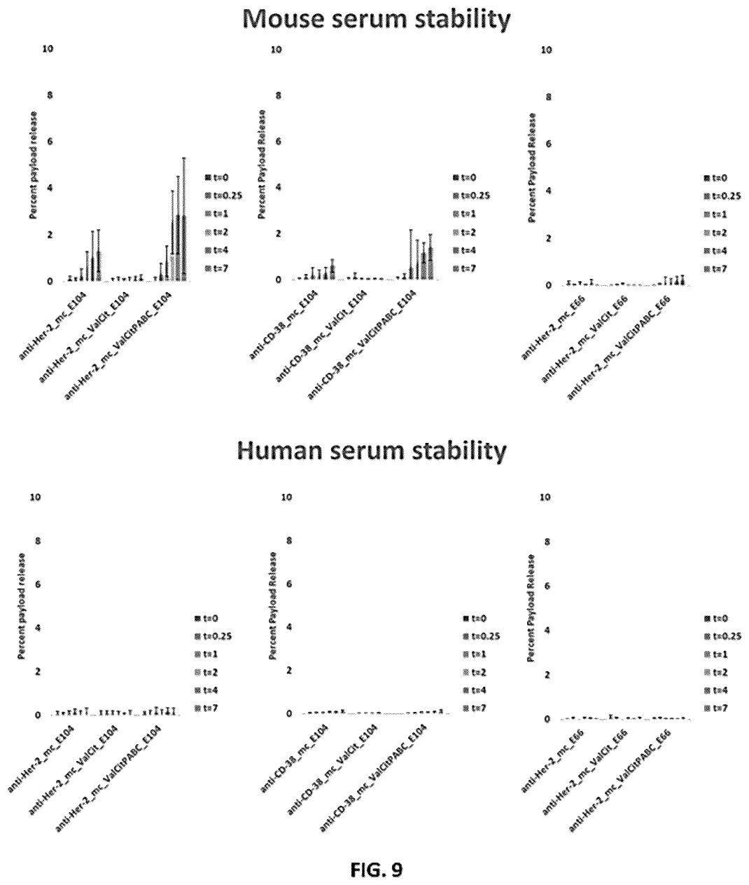

FIG. 8 and FIG. 9 show the results of the stability studies.

### Example 20. Preparation of Additional TLR7/8 Activating Payloads Derived from 1-(4-aminobenzyl)-2-butyl-1H-imidazo[4,5-c]quinolin-4-amine (E104)

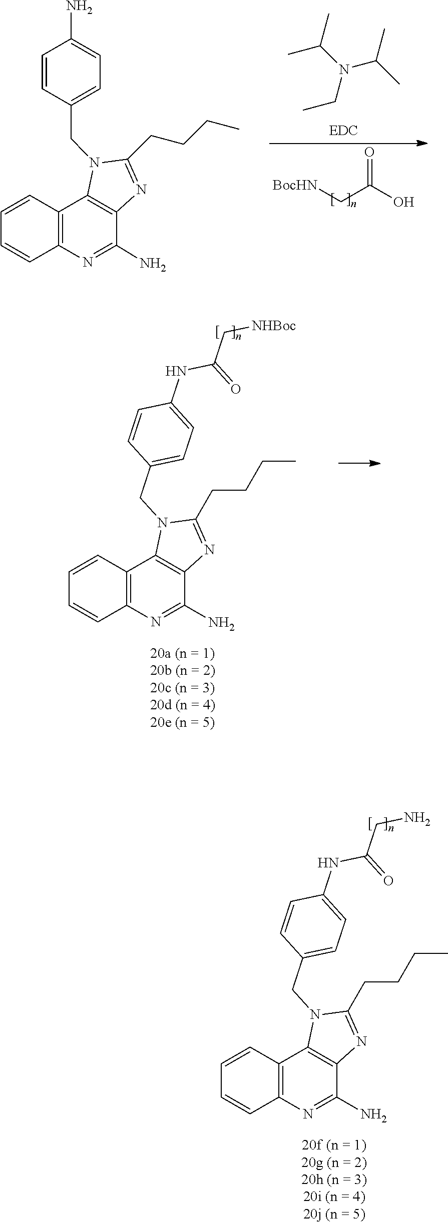

Coupling E104 with boc-glycine, boc-beta-Ala-OH, boc-G-aminobutyric acid, boc-5-aminovaleric acid and boc-6-aminohexanoic acid:

20a. 1-(4-Aminobenzyl)-2-butyl-1H-imidazo[4,5-c]quinolin-4-amine (15.2 mg, 1 Eq, 44.0 mol) was dissolved into DMA (2.0 mL) and treated with DIEA (5.6 mg, 7.6 μL, 0.99 Eq, 44 mol) and boc-glycine (7.7 mg, 1.0 Eq, 44 μmol). EDC (16.9 mg, 2 Eq, 88.0 μmol) was added to the solution, and the reaction was stirred at room temperature for 12 hours. The reaction was purified by prep HPLC to obtain tert-butyl(2-((4-((4-amino-2-butyl-1H-imidazo[4,5-c]quinolin-1-yl)methyl)phenyl)amino)-2-oxoethyl)carbamate. LCMS rt=2.74 min; m/z=503.6 [M+H].

This general procedure was also used to generate the following derivatives:

20b. (3-((4-((4-amino-2-butyl-1H-imidazo[4,5-c]quinolin-1-yl)methyl)phenyl)amino)-3-oxopropyl)carbamate. LCMS rt=2.78 min; m/z=517.7 [M+H]

20c. tert-butyl(4-((4-((4-amino-2-butyl-1H-imidazo[4,5-c]quinolin-1-yl)methyl)phenyl)amino)-4-oxobutyl)carbamate. LCMS rt=2.80 min; m/z=531.6 [M+H]

20d. tert-butyl(5-((4-((4-amino-2-butyl-1H-imidazo[4,5-c]quinolin-1-yl)methyl)phenyl)amino)-5-oxopentyl)carbamate. LCMS rt=2.87 min; m/z=545.6 [M+H]

20e. tert-butyl(6-((4-((4-amino-2-butyl-1H-imidazo[4,5-c]quinolin-1-yl)methyl)phenyl)amino)-6-oxohexyl)carbamate. LCMS rt=2.90 min; m/z=559.7 [M+H].

20f. The Boc-protected material (20a) was dissolved in DCM (400 μL) and treated with TFA (59 g, 40 μL, 56 Eq, 0.52 mmol). After stirring for 1 h, the reaction was concentrated to dryness to obtain the desired product 2-amino-N-(4-((4-amino-2-butyl-1H-imidazo[4,5-c]quinolin-1-yl)methyl)phenyl)acetamide. LCMS rt=0.72 min; m/z=403.5 [M+H].

This general procedure was also used to generate the following derivatives:

20g. 3-amino-N-(4-((4-amino-2-butyl-1H-imidazo[4,5-c]quinolin-1-yl)methyl)phenyl)propanamide. LCMS rt=2.78 min; m/z=417.5 [M+H]

20h. 4-amino-N-(4-((4-amino-2-butyl-1H-imidazo[4,5-c]quinolin-1-yl)methyl)phenyl)butanamide. LCMS rt=2.81 min; m/z=431.3 [M+H]

20i. 5-amino-N-(4-((4-amino-2-butyl-1H-imidazo[4,5-c]quinolin-1-yl)methyl)phenyl)pentanamide. LCMS rt=3.42 min; m/z=445.5 [M+H]

20j. 6-amino-N-(4-((4-amino-2-butyl-1H-imidazo[4,5-c]quinolin-1-yl)methyl)phenyl)hexanamide. LCMS rt=2.33 min; m/z=459.5 [M+H].

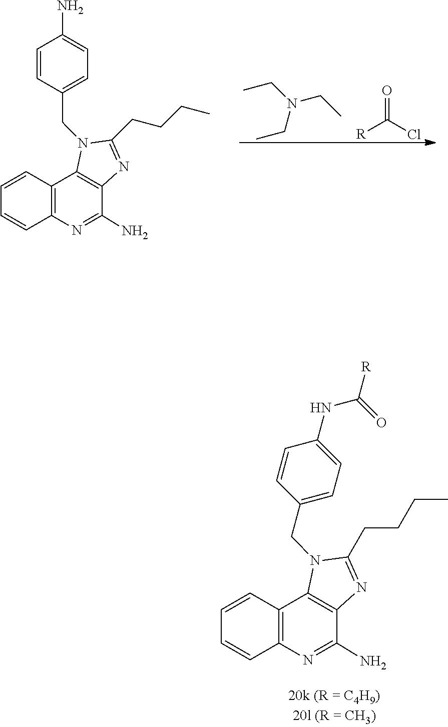

20k. 1-(4-Aminobenzyl)-2-butyl-1H-imidazo[4,5-c]quinolin-4-amine (15.0 mg, 1 Eq, 43.4 μmol) was dissolved in ethyl acetate (400 μL) and treated with triethylamine (5.7 mg, 7.9 μL, 1.3 Eq, 57 μmol). The reaction was chilled on an ice bath. Valeryl chloride (5.8 mg, 5.7 μL, 1.1 Eq, 48 μmol) was then added to pre-chilled ethyl acetate (133 μL), which was subsequently transferred to the reaction dropwise. After bringing the reaction to room temperature and stirring overnight, another 1.3 equivalents of triethylamine and another 1.1 equivalents of valeryl chloride were added to the reaction by the same method as above. The reaction was purified by preparative HPLC to obtain the desired product N-(4-((4-amino-2-butyl-1H-imidazo[4,5-c]quinolin-1-yl)methyl)phenyl)pentanamide. LCMS rt=2.82 min; m/z=430.6 [M+H].

20l. 1-(4-Aminobenzyl)-2-butyl-1H-imidazo[4,5-c]quinolin-4-amine (10.5 mg, 1 Eq, 30.4 μmol) was dissolved in DCM (1.4 mL). Then the acetic anhydride (9.31 mg, 10.2 μL, 3 Eq, 91.2 μmol) was added to the reaction mixture and was stirred at room temperature for 3 hours and monitored by HPLC. This was then purified by prep HPLC to obtain 2.8 mg of the desired product, N-(4-((4-amino-2-butyl-1H-imidazo[4,5-c]quinolin-1-yl)methyl)phenyl)acetamide. LCMS rt=2.71 min; m/z=388.4 [M+H].

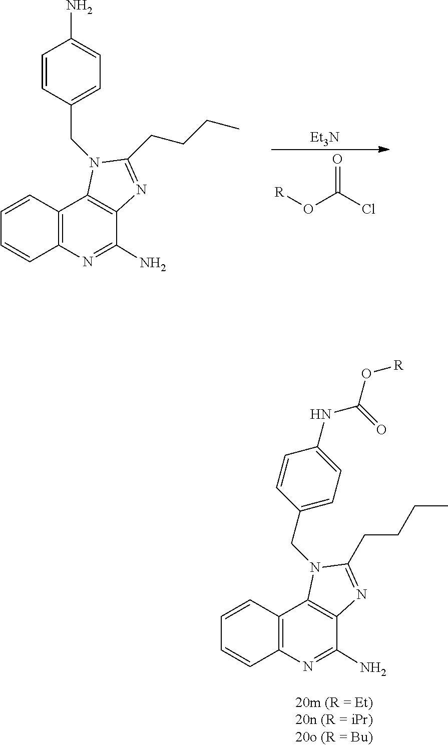

20m. 1-(4-Aminobenzyl)-2-butyl-1H-imidazo[4,5-c]quinolin-4-amine (14.1 mg, 1 Eq, 40.8 μmol) was dissolved in DCM (2.0 mL). Triethylamine (8.26 mg, 11.4 μL, 2 Eq, 82 mol) and ethyl chloroformate (8.9 mg, 7.9 μL, 2.0 Eq, 82.0 μmol) were both added sequentially to the reaction mixture. The reaction was kept at room temperature for 12 hours after which point 2 additional equivalents of ethyl chloroformate were added. After an addition 1 h of stirring, the reaction was purified by prep HPLC to obtain the desired product, ethyl (4-((4-amino-2-butyl-1H-imidazo[4,5-c]quinolin-1-yl)methyl)phenyl)carbamate. LCMS rt=2.77 min; m/z=418.5 [M+H].

This general procedure was also used to generate the following derivatives:

20n. Isopropyl (4-((4-amino-2-butyl-1H-imidazo[4,5-c]quinolin-1-yl)methyl)phenyl)carbamate. LCMS rt=2.91 min; m/z=431.5 [M+H]

20o. Butyl 2-(4-((4-amino-2-butyl-1H-imidazo[4,5-c]quinolin-1-yl)methyl)phenyl)acetate. LCMS rt=3.00 min; m/z=446.6 [M+H].

### Example 21. Preparation of 3-((4-amino-2-butyl-1H-imidazo[4,5-c]quinolin-1yl)methyl)phenol

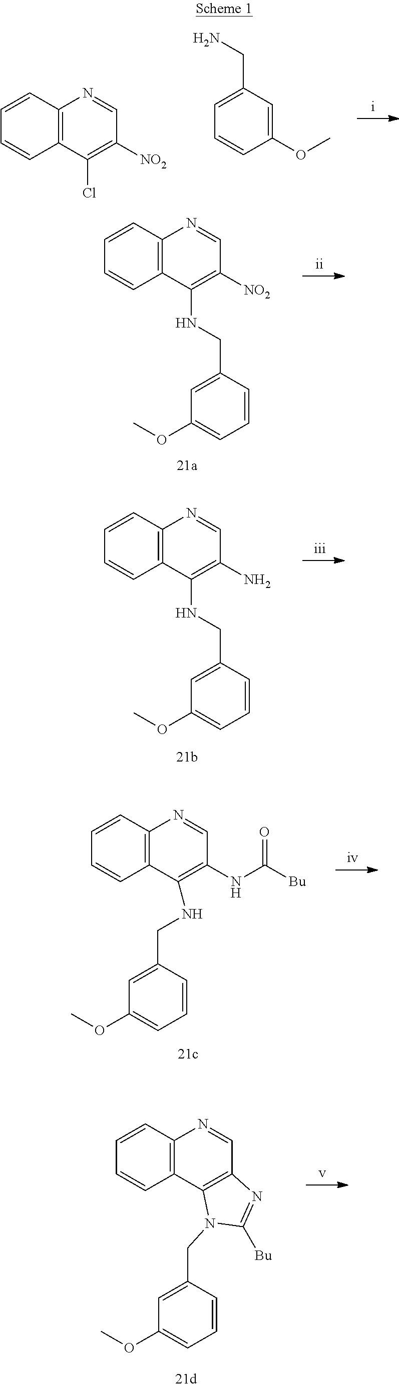

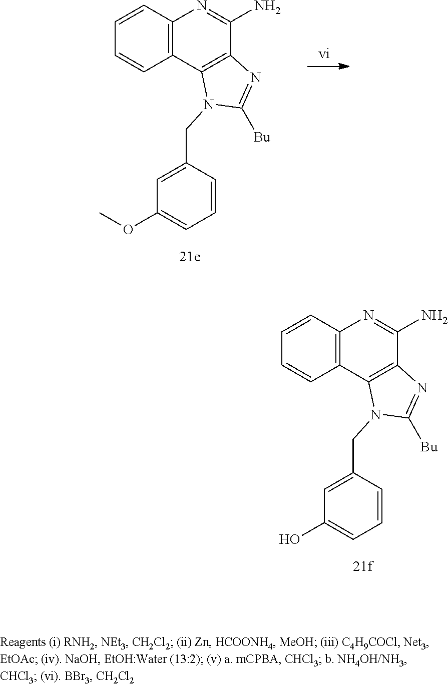

Step 1. 21a. N-(3-methoxybenzyl)-3-nitroquinolin-4-amine: To a suspension of 4-chloro-3-nitroquinoline (1.500 g, 1.0 Eq, 7.20 mmol) in DCM (22.0 mL) was added (3-methoxyphenyl)methanamine (986 mg, 0.92 mL, 1.0 Eq, 7.20 mmol) and triethylamine (1.09 g, 1.50 mL, 1.50 Eq, 10.8 mmol). The mixture was refluxed at 40 C for 1 h. The reaction progress was monitored by UPLC. Complete conversion of the reactants to the desired product was achieved by 60 min, forming a bright yellow suspension. The reaction mixture was cooled to rt, washed with water, filtered and the solid was dried under vacuum to obtain the desired compound (2.18 g) as a bright yellow powder LC/MS rt=3.25 min; m/z=310.1 [M+H].

Step 2. 21b. N4-(3-methoxybenzyl)quinoline-3,4-diamine: To a suspension of the product of step 1 N-(3-methoxybenzyl)-3-nitroquinolin-4-amine (2.179 g, 1.0 Eq, 7.04 mmol) in MeOH (25.0 mL) were added zinc (1.490 g, 4.0 Eq, 28.0 mmol) and Ammonium Chloride (1.80 g, 4.0 Eq, 28.0 mmol). The reaction mixture was stirred at room temperature for 10 min (to give a grey suspension) and monitored by UPLC. Product began forming immediately. After 10 minutes, the reaction mixture was filtered through celite and the solvent was evaporated in vacuo. The residue was dissolved in DCM, partitioned against 1M NaOH, washed with 10% MeOH in DCM (50 ml×3) and dried over MgSO4. The solvent was removed under vacuum to obtain 1.36 g (70%) of the title compound as a black-brown oil. LC/MS rt=2.44; m/z=280.1 [M+H].

Step 3. 21c. N-(4-((3-methoxybenzyl)amino)quinolin-3-yl)pentanamide: To the crude product of step 2 N4-(3-methoxybenzyl)quinoline-3,4-diamine (1.363 g, 1.0 Eq, 4.88 mmol) in anhydrous EtOAc (45.0 mL), cooled to 0° C., was added previously cooled triethylamine (642 mg, 884 μL, 1.3 Eq, 6.34 mmol). The reaction was stirred at rt for 5 mins. Thereafter, Valeryl chloride (883 mg, 868 μL, 1.5 Eq, 7.32 mmol) in EtOAc (15.0 mL) was added dropwise at OC and the reaction mixture was further stirred for 15 min, and monitored by UPLC. The reaction was quenched with ethanol, then concentrated under reduced pressure forming a yellow-brown crystal. LC/MS rt=2.56 min; m/z=364.4 [M+H].

Step 4. 21d. 2-butyl-1-(3-methoxybenzyl)-1H-imidazo[4,5-c]quinoline: The crude product of step 3 N-(4-((3-methoxybenzyl)amino)quinolin-3-yl)pentanamide (1.770 g, 1.0 Eq, 4.88 mmol) was dissolved in EtOH (26.0 mL) and treated with sodium hydroxide (464 mg, 1.0 Eq, 11.6 mmol) in H2O (4.00 mL). The reaction mixture was refluxed at 80 C for 24 h and progress was monitored by UPLC. Upon completion, solution was dissolved in water (75 mL) and partitioned against EtOAc (75 mL). The material was extracted by washing the aqueous layer with EtOAc (75 mL×3). The organic layer was dried over MgSO₄ and evaporated to dryness. The dried amber crude extract (1700 mg) purified by silica gel chromatography using 10% MeOH in DCM. Pure material recovered was an amber resin (856 mg, 51%). LC/MS rt=2.97 min; m/z=346.2 [M+H]. ¹H NMR (400 MHz, DMSO) δ_{H}/ppm 9.21 (s, 1H), 8.11 (d, J=8.5 Hz, 2H), 7.61 (t, J=7.7 Hz, 1H), 7.50 (t, J=7.7 Hz, 1H), 7.21 (t, J=8.0 Hz, 1H), 6.83 (d, J=8.2 Hz, 1H), 6.66 (s, 1H), 6.49 (d, J=7.7 Hz, 1H), 5.93 (s, 2H), 3.67 (s, 3H), 2.96 (t, J=7.5 Hz, 2H), 1.78 (quint, J=7.5 Hz, 2H), 1.40 (sextet, J=7.4 Hz, 2H), 0.88 (t, J=7.4 Hz, 3H).

21e. Step 5A) 2-butyl-1-(3-methoxybenzyl)-1H-imidazo[4,5-c]quinoline-5-oxide: 2-butyl-1-(3-methoxybenzyl)-1H-imidazo[4,5-c]quinoline (156 mg, 1.0 Eq, 450 mmol) in CHCl₃ (2.5 mL) was treated with 3-chlorobenzoperoxoic acid (173 mg, 1.6 Eq, 700 mmol) and stirred at 40° C. for 1 h. The remaining material was used for the next step without further purification nor separation. LC/MS rt=3.26 m/z=362.4 [M+H].

Step 5B) 2-butyl-1-(3-methoxybenzyl)-1H-imidazo[4,5-c]quinoline-4-amine: To the previous reaction containing 2-butyl-1-(3-methoxybenzyl)-1H-imidazo[4,5-c]quinoline-5-oxide (162.8 mg, 1 Eq, 450 umol) in CHCl₃ (2.5 mL) at 50° C. was added 28-38% Ammonium hydroxide 2.2 g, 2.5 mL, 28 Eq, 13 mmol) dropwise followed by the addition of 4-methylbenzenesulfonyl chloride (174 mg, 2 Eq, 920 umol). The mixture was stirred at room temperature for 1 h and monitored by UPLC. Upon completion, the reaction was extracted with chloroform and aqueous bicarbonate, dried over MgSO₄ and solvent removed in vacuo to obtain the 183 mg (112%) of crude product. Material was carried forward to the formation of 3-((4-amino-2-butyl-1H-imidazo[4,5-c]quinoline-1-yl)methyl)phenol without further purification. A fraction was purified by preparative HPLC affording the title compound as a white residue. LC/MS rt=2.78 min; m/z=361.2 [M+H]. ¹H NMR (400 MHz, DMSO) δ_{H}/ppm 7.97 (d, J=7.9 Hz, 1H), 7.80 (d, J=7.8 Hz, 1H), 7.63 (t, J=7.8 Hz, 1H), 7.38 (t, J=7.8 Hz, 1H), 7.24 (t, J=8.0 Hz, 1H), 6.86 (d, J=8.2, 1H), 6.71 (s, 1H), 6.54 (d, J=8.0 Hz, 1H), 5.93 (s, 2H), 3.70 (s, 3H), 2.97 (t, J=7.7 Hz, 2H), 1.72 (quint, J=7.6 Hz, 2H), 1.38 (sextet, J=7.4 Hz, 2H), 0.87 (t, J=7.4 Hz, 3H).

Step 6. 21f. 3-((4-amino-2-butyl-1H-imidazo[4,5-c]quinolin-1-yl)methyl)phenol: To a suspension of crude extract 2-butyl-1-(3-methoxybenzyl)-1H-imidazo[4,5-c]quinoline-4-amine (105.5 mg, 1.0 Eq, 293 μmol) in DCM (1.00 mL) cooled to OC under nitrogen, was added BBr₃ (220.0 mg, 83 uL, 3.0 Eq, 878 μmol) in DCM (0.70 mL) dropwise. The mixture was stirred at OC for 5 minutes before being brought up to rt and monitored for 1.5 h by UPLC. Upon completion, the reaction was quenched by pouring over iced water. This solution was extracted with DCM and aqueous bicarbonate, washing the bicarbonate with DCM (10 mL×2), dried over MgSO4, and evaporated to dryness. The residue was purified by preparative HPLC affording the title compound as a fine white powder (35.0 mg, 35%) LC/MS: Retention time=2.57 min: m/z 347.2 [M+1]. ¹H NMR (400 MHz, DMSO) δ_{H}/ppm 7.97 (d, J=7.8 Hz, 1H), 7.80 (d, J=8.0 Hz, 1H), 7.64 (t, J=7.8 Hz, 1H), 7.39 (t, J=7.8 Hz, 1H), 7.14 (t, J=7.8 Hz, 1H), 6.66 (d, J=8.1 Hz, 1H), 6.52 (d, J=7.9 Hz, 1H), 6.39 (s, 1H), 5.88 (s, 2H), 2.97 (t, J=7.7 Hz, 2H), 1.72 (quint, J=7.6 Hz, 2H), 1.39 (sextet, J=7.4 Hz, 2H), 0.87 (t, J=7.3, 3H)

### Example 22: Preparation of 4-((4-amino-2-butyl-1H-imidazo[4,5-c]quinolin-1-yl)methyl)phenol

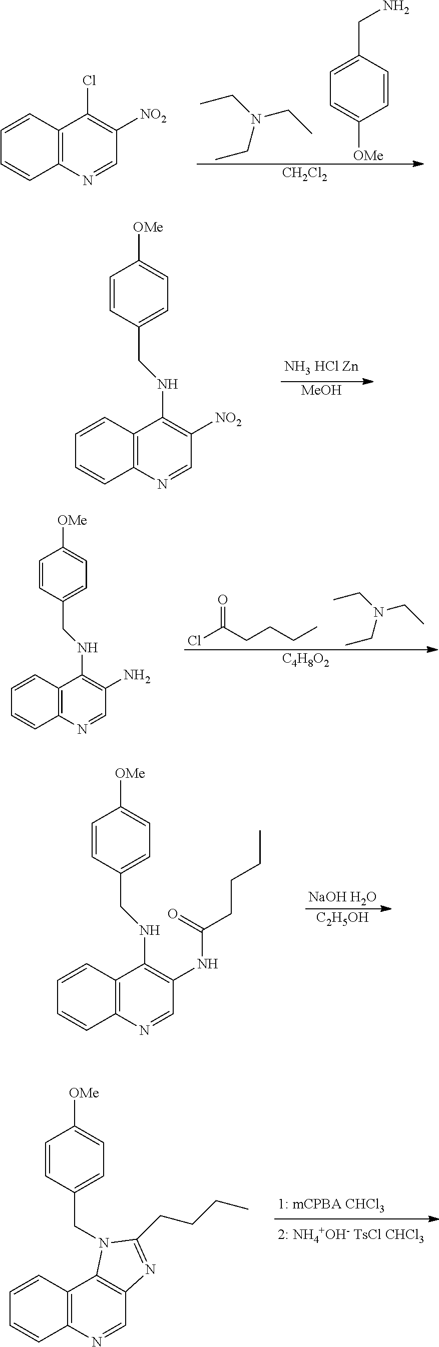

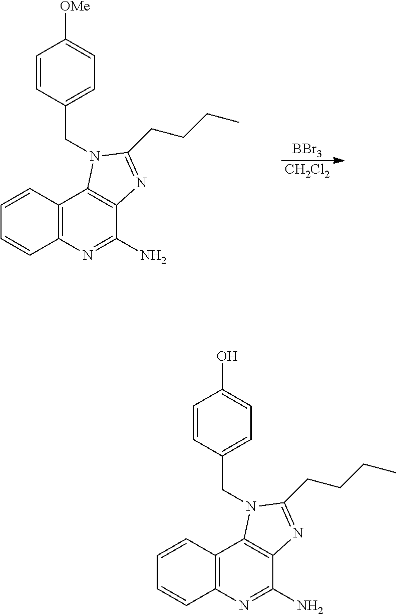

Step 1: 22a. 4-Chloro-3-nitroquinoline (1003.4 mg, 1 Eq, 4.8102 mol) was dissolved in DCM (15.0 mL) and treated with (4-methoxyphenyl)methanamine (725 mg, 690 L, 1.10 Eq, 5.28 mmol) and then triethylamine (973.48 mg, 1.34 mL, 2 Eq, 9.6203 mmol). The reaction was heated to 30° C. and left to stir for 1 hour while being monitored by UPLC. The reaction was then cooled, concentrated to dryness, and triturated with water. The product was isolated by vacuum filtration giving N-(4-methoxybenzyl)-3-nitro quinolin-4-amine (1249.4 mg, 4.0391 mmol, 83.969% yield) as a bright yellow powder. LCMS rt=3.23 min; m/z=310.1 [M+H].

Step 2: 22b. N-(4-methoxybenzyl)-3-nitro quinolin-4-amine (501.2 mg, lEq, 1.620 mmol) was suspended in Methanol (30.0 mL). The solution was stirred and ammonium chloride (871.8 mg, 10.06 Eq, 16.30 mmol) was added. This was then put into an ice bath and treated with Zinc (1062.8 mg, 10.03 Eq, 16.30 mmol). The reaction was kept for a total of 25 minutes and was monitored by UPLC, giving N4-(4-methoxybenzyl)quinoline-3,4-diamine 534.3 mg of crude product. The crude product was dissolved in 1:1 methanol:DCM and filtered through celite in order to remove the excess Zinc. The solvent was then evaporated and the product was re-dissolved in 10% methanol in DCM (40.0 mL) and washed with 1M NaOH (40.0 mL). This was repeated 3 times and then the organic layer was dried using MgSO4. This was then dried to recover N4-(4-methoxybenzyl)quinoline-3,4-diamine (307.6 mg, 57.6% recovery). LCMS rt=1.64 min; m/z=280.4 [M+H].

Steps 3 and 4: 22d. 307.6 mg of N4-(4-methoxybenzyl)quinoline-3,4-diamine was brought forward and dissolved in Ethyl Acetate (5 mL). Triethylamine (144.9 mg, 200 μL, 1.3 Eq, 1.432 mmol) was added and the reaction was cooled on ice. Valeryl chloride (152 mg, 150 μL, 1.15 Eq, 1.26 mmol) was combined with the 1.25 mL of chilled ethyl acetate, which was then added dropwise to the reaction mixture. The resulting mixture stirred in the ice bath for 20 minutes while the reaction was monitored by HPLC. Upon completion, the reaction was concentrated to dryness for a final weight of 575.7 mg. LCMS rt=2.59 min; m/z=364.4 [M+H]. The crude product (575.5 mg) of N-(4-((4-methoxybenzyl)amino)quinolin-3-yl)pentanamide was then dissolved in ethanol (13 mL). In a separate container NaOH (127.4 mg, 2.893 Eq, 3.185 mmol) was dissolved in water (1.8 mL). Both the reaction and the NaOH solution were heated to 80° C. The sodium hydroxide was then added to the reaction. The reaction was heated at 80° C. for three and a half hours at reflux and monitored by UPLC. 25 mL of water was then added to the reaction and this was partitioned against DCM. The organic layer was washed three times with water and then dried with Magnesium sulfate. The crude material (342.4 mg) was further purified using silica gel chromatography using a 0%->10% Methanol in DCM gradient. The isolated fractions were concentrated to dryness giving 153.8 mg of pure 2-butyl-1-(4-methoxybenzyl)-1H-imidazo[4,5-c]quinoline recovered (44.9%). LCMS rt=2.95 min; m/z=346.3 [M+H].

Step 5: 22e. 2-butyl-1-(4-methoxybenzyl)-1H-imidazo[4,5-c]quinoline (141.4 mg, 1 Eq, 409.3 μmol) was dissolved in chloroform (2.5 mL) and treated with 3-chlorobenzoperoxoic acid (215.6 mg, 3.052 Eq, 1.249 mmol). The mixture was vortexed and heated to 40° C. and stirred for 1 hour, resulting in the formation of 2-butyl-1-(4-methoxybenzyl)-1H-imidazo[4,5-c]quinoline 5-oxide. LCMS rt=3.24 min; m/z=362.3 [M+H]. The reaction was cooled back down to room temperature and then stirred vigorously before adding ammonium hydroxide (405 mg, 460 μL, 28.2 Eq, 11.5 mmol). After stirring for 1 h, 4-methylbenzenesulfonyl chloride (161.2 mg, 2.006 Eq, 845.6 μmol) was added to the reaction. After 30 minutes, the reaction was concentrated to dryness and re-dissolved in 40 mL of chloroform. The organic solution was washed with 40 mL of sodium bicarbonate followed by 40 mL of brine, resulting in 229.2 mg of recovered crude product. A portion of this crude material (70.6 mg) was then further purified by prep HPLC resulting in 31.9 mg of the pure methoxy E104 material, 2-butyl-1-(4-methoxybenzyl)-1H-imidazo[4,5-c]quinolin-4-amine. LCMS rt=2.77 min; m/z=361.5 [M+H].

Step 6: 22f. 23.5 mg of 2-butyl-1-(4-methoxybenzyl)-1H-imidazo[4,5-c]quinolin-4-amine was dissolved in DCM (500 μL) and put under nitrogen. The temperature was brought down in an ice bath. 20 μL of BBr3 was diluted with 500 μL of DCM and this was then added to the reaction dropwise. The reaction was warmed to room temperature and left for 2 hours while being monitored by HPLC. The reaction was left at rt overnight after which time three more equivalents of BBr3 were added and the reaction was stirred for an addition 2 h. The mixture was carefully quenched with 1 mL water and 1 mL of sodium bicarbonate. The organic layer was dried over magnesium sulfate giving 5.2 mg of the impure material which was then further purified by prep HPLC giving 3.3 mg of pure 4-((4-amino-2-butyl-1H-imidazo[4,5-c]quinolin-1-yl)methyl)phenol. LCMS rt=2.13 min; m/z=347.4 [M+H].

### Example 23: Preparation of 1-(3-aminobenzyl)-2-butyl-1H-imidazo[4,5-c]quinolin-4-amine

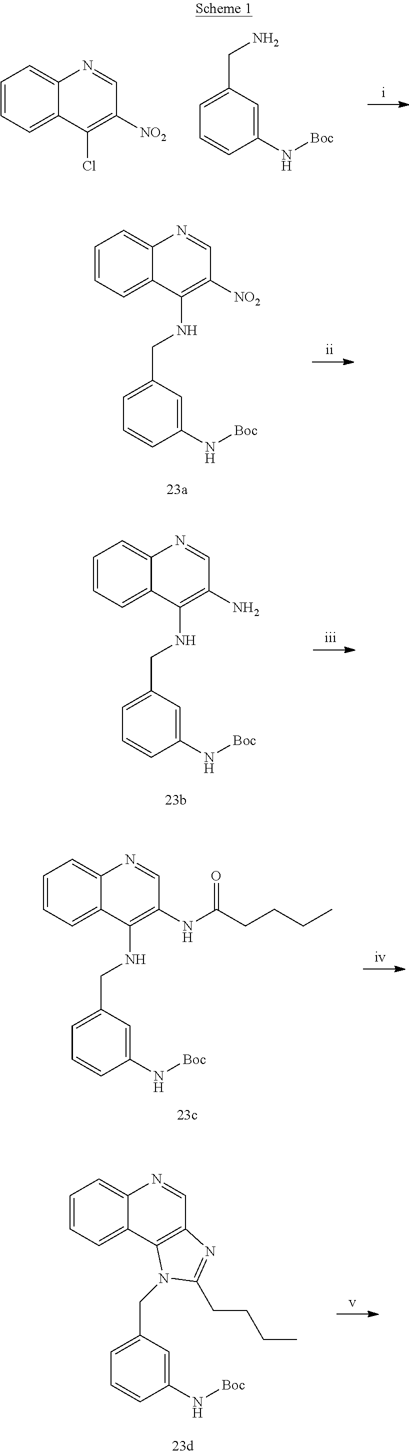 [SMILES: O=[N+]([O-])c1cnc2ccccc2c1Cl ; CC(C)(C)OC(=O)Nc1cccc(CN)c1 ; CC(C)(C)OC(=O)Nc1cccc(CNc2c([N+](=O)[O-])cnc3ccccc23)c1 ; CC(C)(C)OC(=O)Nc1cccc(CNc2c(N)cnc3ccccc23)c1 ; CCCCC(=O)Nc1cnc2ccccc2c1NCc1cccc(NC(=O)OC(C)(C)C)c1 ; CCCCc1nc2cnc3ccccc3c2n1Cc1cccc(NC(=O)OC(C)(C)C)c1]

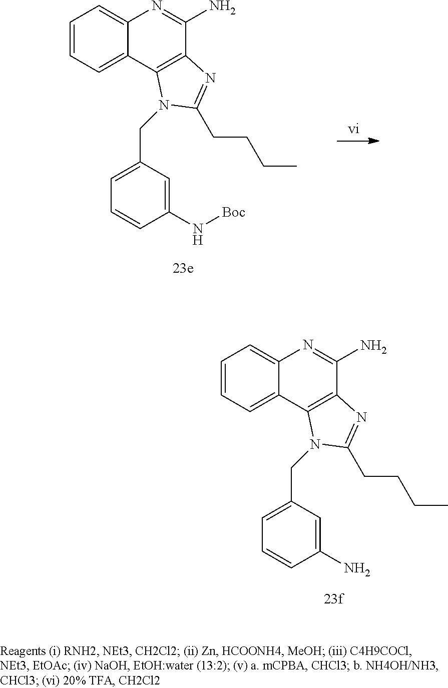 [SMILES: CCCCc1nc2c(N)nc3ccccc3c2n1Cc1cccc(NC(=O)OC(C)(C)C)c1 ; CCCCc1nc2c(N)nc3ccccc3c2n1Cc1cccc(N)c1]

Step 1. 23a. tert-butyl (3-(((3-nitroquinolin-4-yl)amino)methyl)phenyl)carbamate: To a solution of tert-butyl (3-(aminomethyl)phenyl)carbamate (1077 mg, 1 Eq, 4.85 mmol) in DCM (27.0 mL) was added 4-chloro-3-nitroquinoline (1005 mg, 1 Eq, 4.82 mmol) and triethylamine (975 mg, 1.34 mL, 2 Eq, 9.64 mmol). The mixture was brown and became yellow once the triethylamine was added. The mixture brought to reflux at 40° C. for 1 h. The reaction process was monitored by HPLC. Near-complete conversion of the reactants to the desired product was achieved by 60 min forming a yellow precipitate. The reaction was cooled to room temperature and concentrated to dryness under reduced pressure forming yellow crystals. The crystals were suspended in water (140 mL), filtered under vacuum through a sintered funnel and dried in a desiccator to obtain the desired compound. (1550.2 mg, 81.54% yield) HPLC rt=3.50 min (polar gradient); m/z=394.43 [M+H].

Step 2. 23b. tert-butyl (3-(((3-aminoquinolin-4-yl)amino)methyl)phenyl)carbamate: A suspension of the Tert-butyl (3-(((3-nitroquinolin-4-yl)amino)methyl)phenyl)carbamate (650 mg, 1 Eq, 1.65 mmol) in MeOH (20 mL) was treated with a pre-cooled suspension of zinc (1099 mg, 10.2 Eq, 16.81 mmol) and ammonium chloride (899.8 mg, 10.2 Eq, 16.82 mmol) in MeOH (6 mL). The mixture was stirred at 0° C. for 20 min and monitored by HPLC. The mixture rapidly turned to a grey/green suspension. After 20 min the reaction mixture was filtered through celite and the solvent was evaporated in vacuo. The residue was dissolved in DCM, washed with water, and back-extracted into DCM. The organic layer was dried over MgSO4. The solvent was removed under vacuum to obtain the desired compound (549.2 mg, 91.4%). HPLC rt=2.66 min (polar gradient); m/z=365.45 [M+H].

Step 3. 23c. tert-butyl (3-(((3-pentanamidoquinolin-4-yl)amino)methyl)phenyl)carbamate: The crude tert-butyl (3-(((3-aminoquinolin-4-yl)amino)methyl)phenyl)carbamate (549.2 mg, 1.0 Eq, 1.51 mmol) in anhydrous EtOAc (16 mL), cooled to 0° C., was added to triethylamine (198.2 mg, 273 μL, 1.3 Eq, 1.96 mmol) also at 0° C. This mixture was stirred for 10 min. Next, Valeryl chloride (236.2 mg, 232.5 μL, 1.3 Eq, 1.96 mmol) in EtOAc (5 mL) was added dropwise at 0° C. to the reaction mixture. The mixture was stirred for 40 min and monitored by HPLC. The solution was concentrated to dryness under reduced pressure and carried forward without purification. HPLC rt=2.79 min (polar gradient) m/z=449.57 [M+H].

Step 4. 23d. tert-butyl (3-((2-butyl-1H-imidazo[4,5-c]quinoline-1-yl)methyl)phenyl)carbamate: The crude product from the previous step (Tert-butyl (3-(((3-pentanamidoquinolin-4-yl)amino)methyl)phenyl)carbamate, 676 mg, 1 Eq, 1.51 mmol) was dissolved in EtOH (9 mL) and treated with sodium hydroxide (121 mg, 2 Eq, 3.01 mmol) in H2O (1.38 mL). The mixture was refluxed for 4 h and was monitored by HPLC. Water was directly added to the reaction solution and the product was extracted with DCM. The organic layer was dried over MgSO4 and evaporated to dryness. The amber-red product was purified using silica gel chromatography. A 11 min gradient of 100% to 5% MeOH in DCM was used with the resulting eluent concentrated to dryness under reduced pressure. (647.3 mg, 99.8%) HPLC rt=3.15 min (polar gradient) m/z=431.55 [M+H].

Step 5. 23e. a. 1-(3-((tert-butoxycarbonyl)amino)benzyl)-2-butyl-1H-imidazo[4,5-c]quinoline 5-oxide: 3-Chlorobenzoperoxoic acid (382 mg, 2.896 Eq, 1.55 mmol) was added to a solution of tert-butyl (3-((2-butyl-1H-imidazo[4,5-c]quinoline-1-yl)methyl)phenyl)carbamate (230.4 mg, 1.0 Eq, 0.535 mmol) in CHCl2 (3 mL). The reaction mixture was stirred at 40° C. for 1 h. The crude solution was dried under a stream of air and brought forward to the next step without workup. HPLC rt=3.50 min (Polar gradient) m/z=447.55 [M+H].

b. Tert-butyl (3-((4-amino-2-butyl-1H-imidazo[4,5-c]quinoline-1-yl)methyl)phenyl)carbamate: 1-(3-((tert-butoxycarbonyl)amino)benzyl)-2-butyl-1H-imidazo[4,5-c]quinoline 5-oxide (239.0 mg, 1 Eq, 0.535 mmol) from the previous step was re-dissolved in CHCl3 (10 mL) and warmed to 50° C. Ammonium hydroxide (20%) (2.81 g, 30 Eq, 16.0 mmol) was added dropwise and the mixture was left to stir for 1 h at 50° C. 4-Methylbenzenesulfonyl chloride (204.1 mg, 2 Eq, 1.070 mmol) was added to this mixture and the resulting reaction was stirred at 50° C. for 4 h. The reaction was then diluted with CHCl3 (40 mL) and the organic fraction was washed with aqueous bicarbonate, then washed with brine. The organic layer was dried with MgSO4 dried under reduced pressure. The crude product was then purified by silica gel chromatography (0->20% DCM/MeOH). The pure fractions were dried under reduced pressure giving 164.7 mg (69.1%) of the title compound. HPLC rt=2.94 min (polar gradient) m/z=446.57 [M+H].

Step 6. 23f. 1-(3-aminobenzyl)-2-butyl-1H-imidazo[4,5-c]quinoline-4-amine: To a solution of Tert-butyl (3-((4-amino-2-butyl-1H-imidazo[4,5-c]quinoline-1-yl)methyl)phenyl)carbamate (30 mg, 67 μmol) in DCM was added TFA (78 μL, 15 eq). The reaction mixture was stirred at 23 C for 3 h. The resulting product mixture was dried under reduced pressure resulting in the desired material as a darker red-amber crystal. (16.3 mg, 71%) HPLC rt=2.61 min (polar gradient) m/z=346.45 [M+H].

### Example 24: Preparation of TRL Activating Linker-Payloads

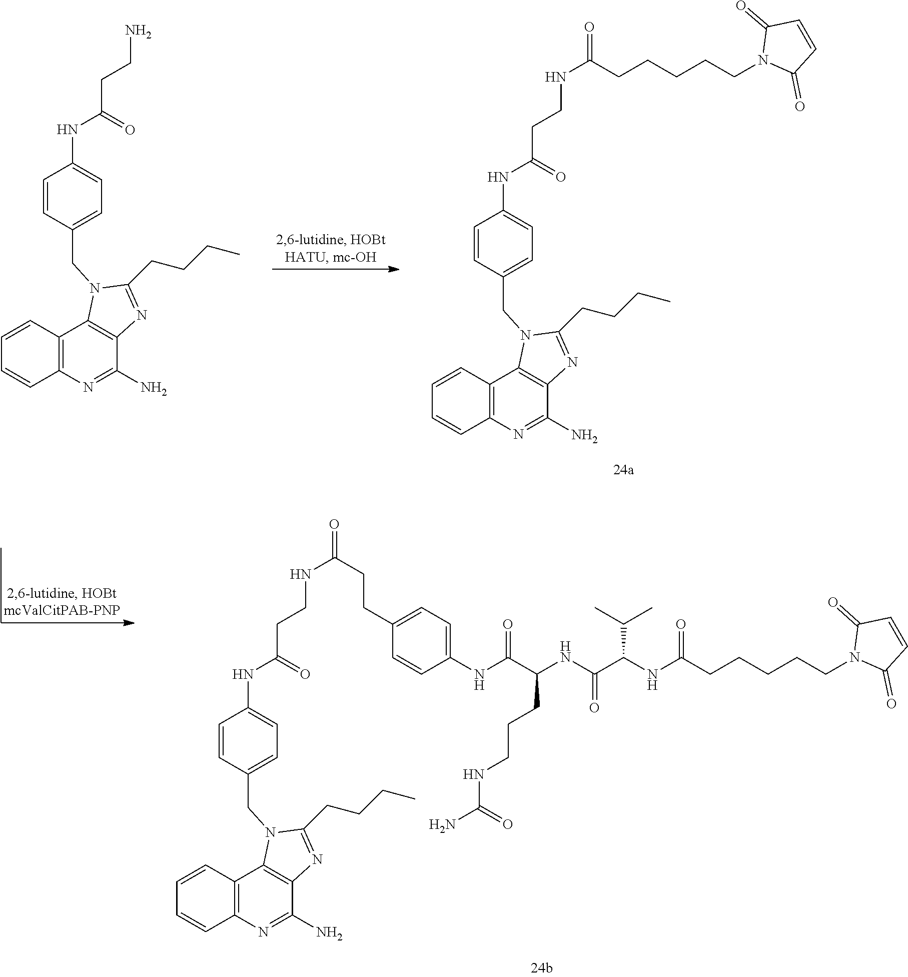 [SMILES: CCCCc1nc2c(N)nc3ccccc3c2n1Cc1ccc(NC(=O)CCN)cc1 ; CCCCc1nc2c(N)nc3ccccc3c2n1Cc1ccc(NC(=O)CCNC(=O)CCCCCN2C(=O)C=CC2=O)cc1 ; CCCCc1nc2c(N)nc3ccccc3c2n1Cc1ccc(NC(=O)CCNC(=O)CCc2ccc(NC(=O)[C@H](CCCNC(N)=O)NC(=O)[C@@H](NC(=O)CCCCCN3C(=O)C=CC3=O)C(C)C)cc2)cc1]

4-((S)-2-((S)-2-(6-(2,5-dioxo-2,5-dihydro-1H-pyrrol-1-yl)hexanamido)-3-methylbutanamido)-5-ureidopentanamido)benzyl (3-((4-((4-amino-2-butyl-1H-imidazo[4,5-c]quinolin-1-yl)methyl)phenyl)amino)-3-oxopropyl)carbamate (24a): 3-amino-N-(4-((4-amino-2-butyl-1H-imidazo[4,5-c]quinolin-1-yl)methyl)phenyl)propanamide (14.2 mg, 1 Eq, 34.1 μmol) was dissolved in DMA (1500 L). Then mcValCitPABC-PNP (25.2 mg, 1 Eq, 34.1 μmol), 2,6-lutidine (7.31 mg, 2 Eq, 68.2 μmol) and HOBt (6.3 mg, 1.2 Eq, 41 μmol) were added. This was stirred over the span of a couple days at room temperature and monitored by HPLC. This was then purified by prep HPLC to obtain 10.3 mg of the title product. HPLC rt=2.79 min, m/z=1015.8 [M+H].

N-(3-((4-((4-amino-2-butyl-1H-imidazo[4,5-c]quinolin-1-yl)methyl)phenyl)amino)-3-oxopropyl)-6-(2,5-dioxo-2,5-dihydro-1H-pyrrol-1-yl)hexanamide (24b): 3-amino-N-(4-((4-amino-2-butyl-1H-imidazo[4,5-c]quinolin-1-yl)methyl)phenyl)propanamide (14.7 mg, 1 Eq, 35.3 μmol) was dissolved in DMA (1500 μL) and HOBt (7.7 mg, 1.4 Eq, 50 μmol), 2,6-lutidine (11.3 mg, 12.3 μL, 3 Eq, 106 μmol) and 6-maleimidohexanoic acid (7.5 mg, 1 Eq, 36 μmol) were added. Then finally HATU (17.4 mg, 1.3 Eq, 45.9 μmol) was added. This was stirred at room temperature for 1.5 hours and monitored by HPLC. This was then purified by HPLC to obtain 7.6 mg of the title product. HPLC rt=2.96 min, m/z=610.5 [M+H].

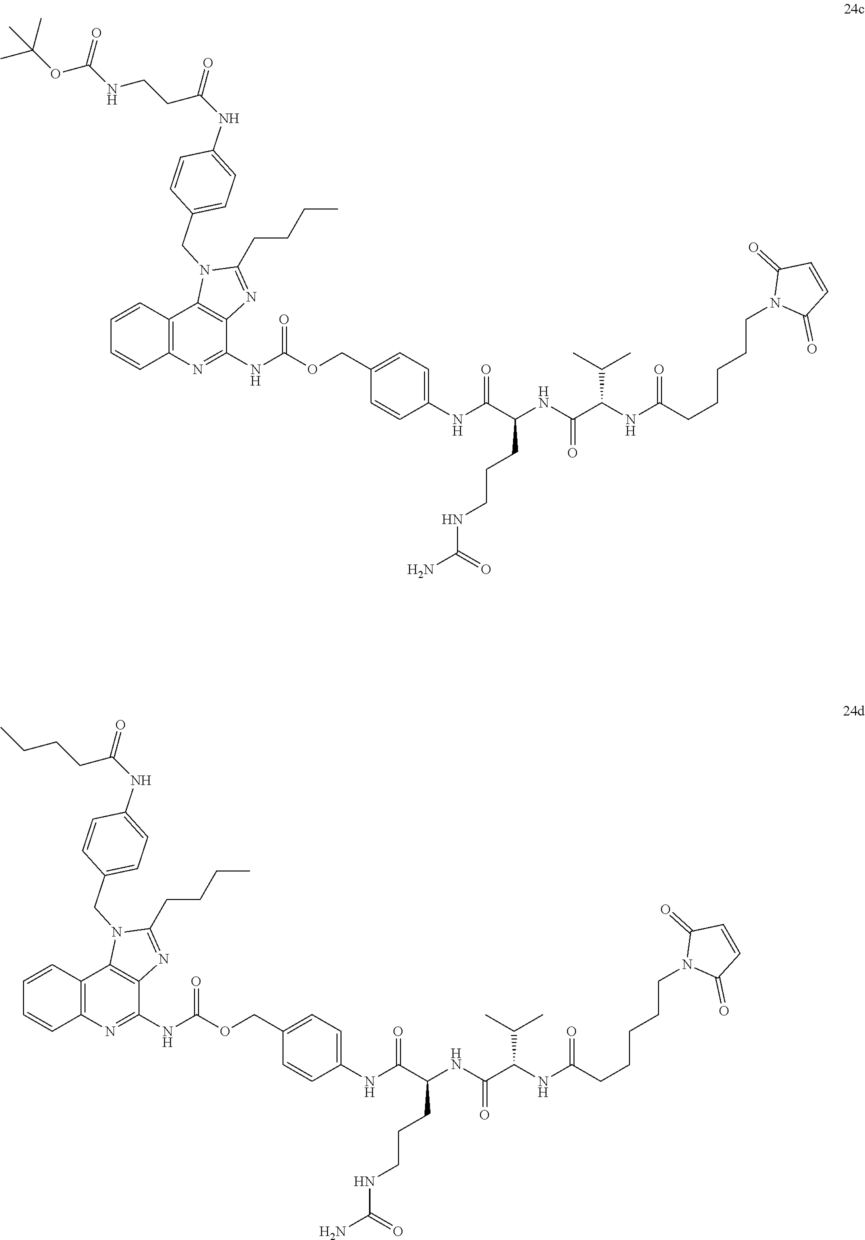 [SMILES: CCCCc1nc2c(NC(=O)OCc3ccc(NC(=O)[C@H](CCCNC(N)=O)NC(=O)[C@@H](NC(=O)CCCCCN4C(=O)C=CC4=O)C(C)C)cc3)nc3ccccc3c2n1Cc1ccc(NC(=O)CCNC(=O)OC(C)(C)C)cc1 ; CCCCC(=O)Nc1ccc(Cn2c(CCCC)nc3c(NC(=O)OCc4ccc(NC(=O)[C@H](CCCNC(N)=O)NC(=O)[C@@H](NC(=O)CCCCCN5C(=O)C=CC5=O)C(C)C)cc4)nc4ccccc4c32)cc1]

The general procedures outlined above were also used to generate:

4-((S)-2-((S)-2-(6-(2,5-dioxo-2,5-dihydro-1H-pyrrol-1-yl)hexanamido)-3-methylbutanamido)-5-ureidopentanamido)benzyl(1-(4-(3-((tert-butoxycarbonyl)amino)propanamido)benzyl)-2-butyl-1H-imidazo[4,5-c]quinolin-4-yl)carbamate (24c) LCMS rt=3.10 min; m/z=1116.3 [M+H]

4-((S)-2-((S)-2-(6-(2,5-dioxo-2,5-dihydro-1H-pyrrol-1-yl)hexanamido)-3-methylbutanamido)-5-ureidopentanamido)benzyl (2-butyl-1-(4-pentanamidobenzyl)-1H-imidazo[4,5-c]quinolin-4-yl)carbamate (24d) LCMS rt=3.15 min; m/z=1029.2 [M+H].

Additional analogs such as 24e-24i (below) are prepared by slight modifications of the above procedures.

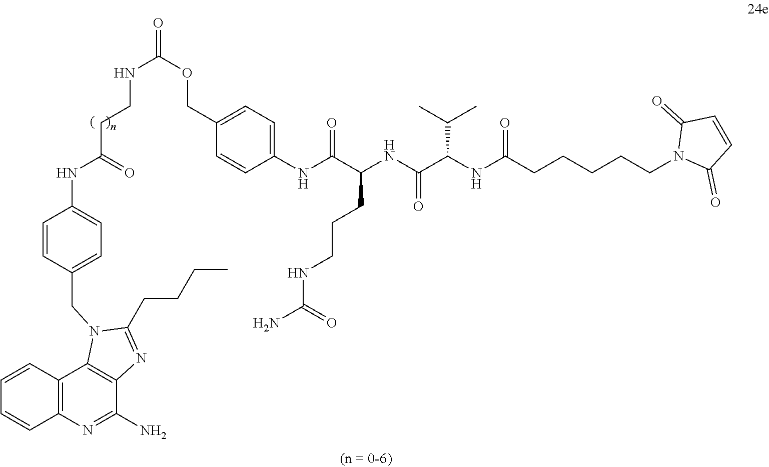

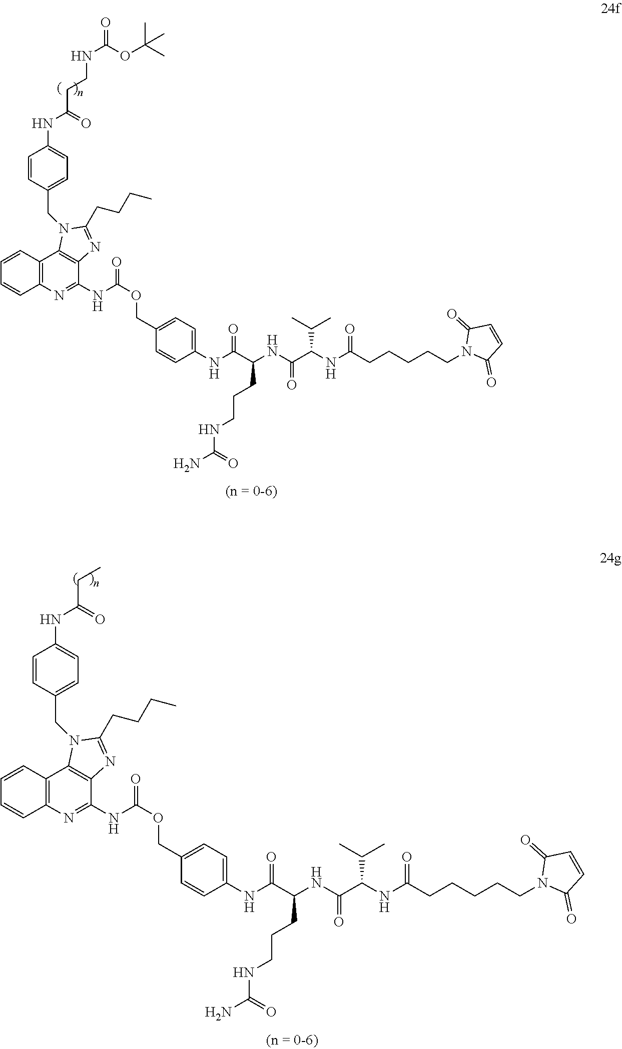

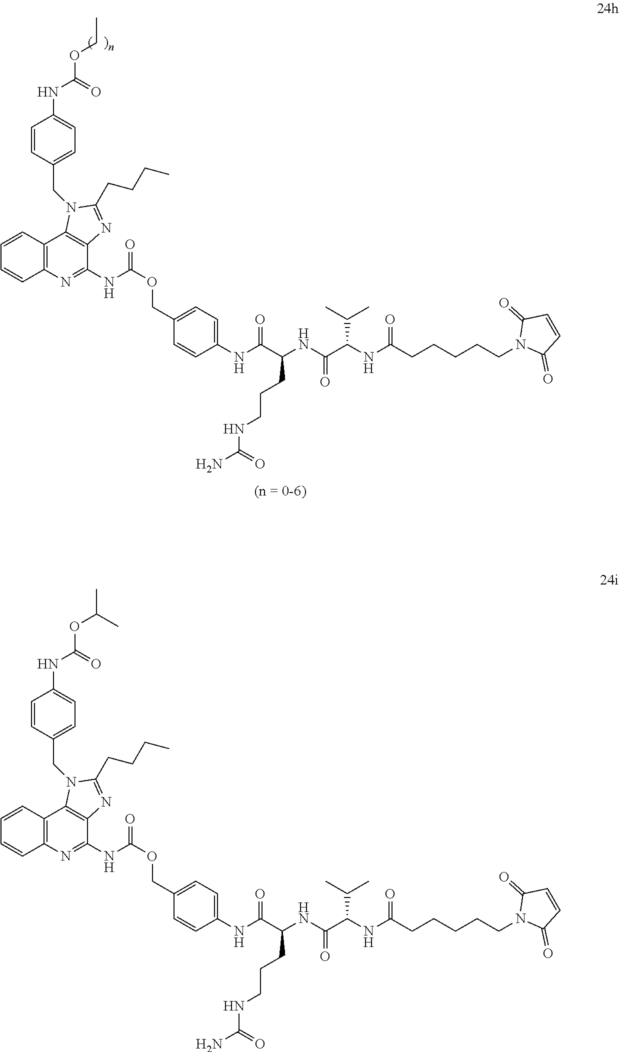

### Example 25: Preparation of (S)—N1-(4-((4-amino-2-butyl-1H-imidazo[4,5-c]quinolin-1-yl)methyl)phenyl)-2-(6-(2,5-dioxo-2,5-dihydro-1H-pyrrol-1-yl)hexanamido)succinimide (mcAsn-E104)

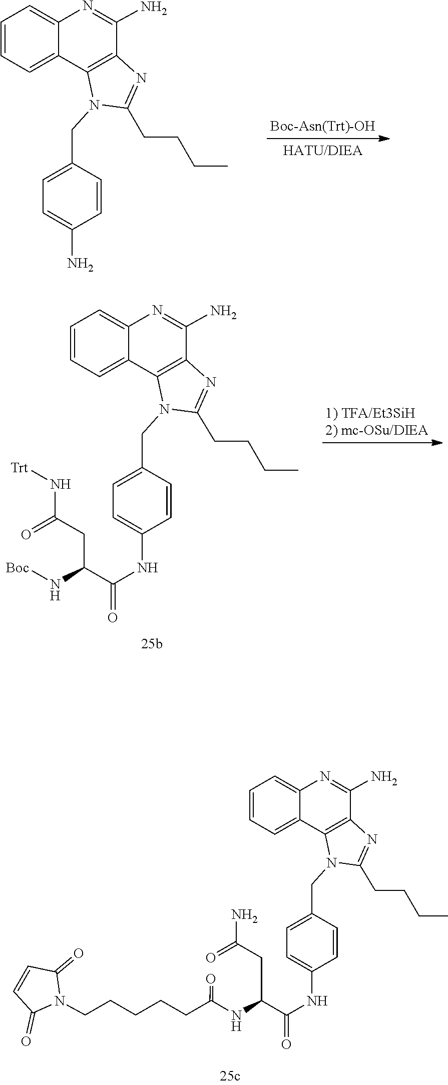

Step 1: tert-butyl (S)-(1-((4-((4-amino-2-butyl-1H-imidazo[4,5-c]quinolin-1-yl)methyl)phenyl)amino)-1,4-dioxo-4-(tritylamino)butan-2-yl)carbamate: To a 4 ml glass LC/MS vial, 750 ul of DMF was added followed by 20 mg of 1-(4-aminobenzyl)-2-butyl-1H-imidazo[4,5-c]quinolin-4-amine (E104) (1.4 eq), 20 mg of Boc-Asn(Trt)-OH (1 eq), 10 mg of HOBt (1 eq) and 28 mg of HATU (1.8 eq). After briefly vortexing, DIPEA (4 eq) was added to initiate the reaction. The solution was a clear yellow and was allowed to stand for 30 hours and then purified by prep HPLC using a water and ACN+TFA mobile phase and the normal gradient, giving 4.3 mg (9.3%) of the title compound.

Step 2: The material from step 1 was treated with 950 ul of TFA and immediately the reaction turned to a vibrant yellow color. After 2 minutes, triethylsilane was added resulting in the dissipation of the yellow color. After 3 minutes, LC/MS indicated that the deprotection was complete. The product was dried over air to remove TFA and used directly in the next step.

Step 3: (S)—N1-(4-((4-amino-2-butyl-1H-imidazo[4,5-c]quinolin-1-yl)methyl)phenyl)-2-(6-(2,5-dioxo-2,5-dihydro-1H-pyrrol-1-yl)hexanamido)succinimide: The crude material from step 2 was dissolved into 500 ul of DMF and transferred to a 1 ml glass LC/MS vial. 1.5 eq of mcOSu was added followed by 10 eq of DIPEA (to neutralize excess TFA). LC/MS was run to monitor the reaction progress. The title product was purified by preparative HPLC using the ACN+Water with TFA additive under the normal gradient resulting in 3.2 mg (90%) of the title compound. A stock solution was prepared by dissolving the solid into ˜950 ul of DMA forming a 5 mM stock solution. HPLC RT=2.41 min; M+H=774.5 Da.

Additional analogs such as 25d, 25e, 25f, 25g, and 25h (below) are prepared by slight modifications of the above procedures.

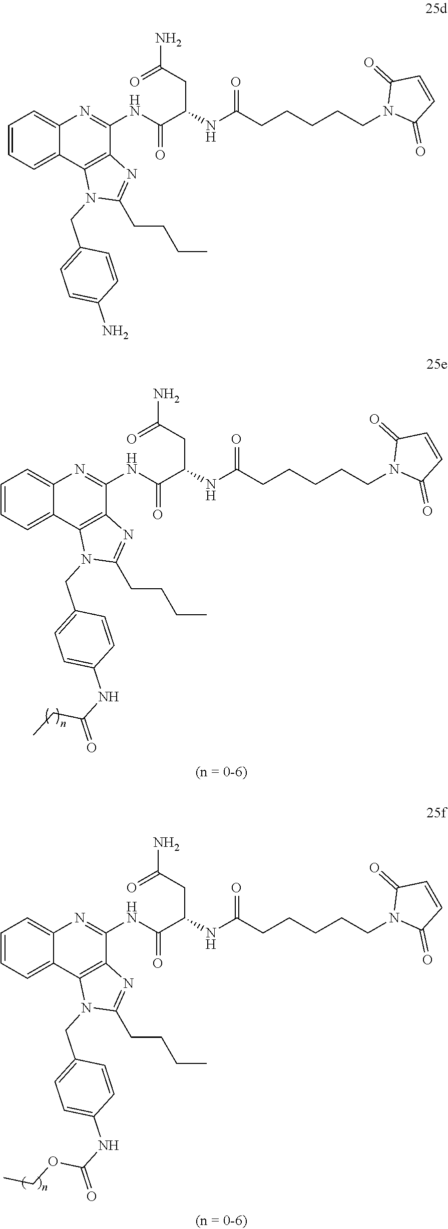

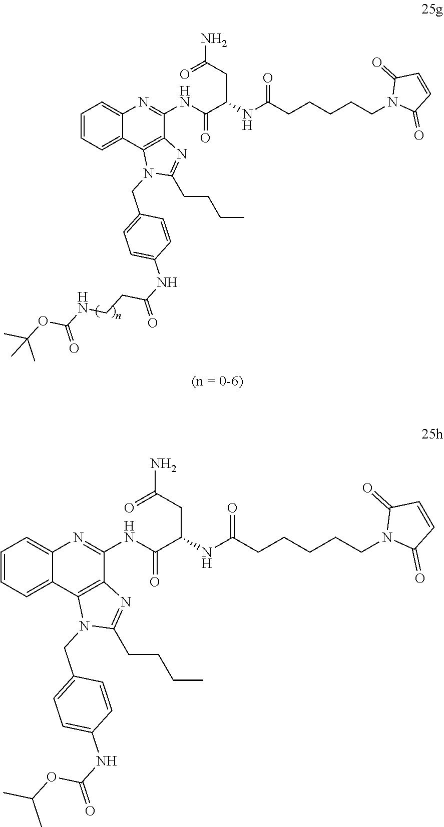

### Example 26: Preparation of Additional TLR Agonist Linker-Payloads

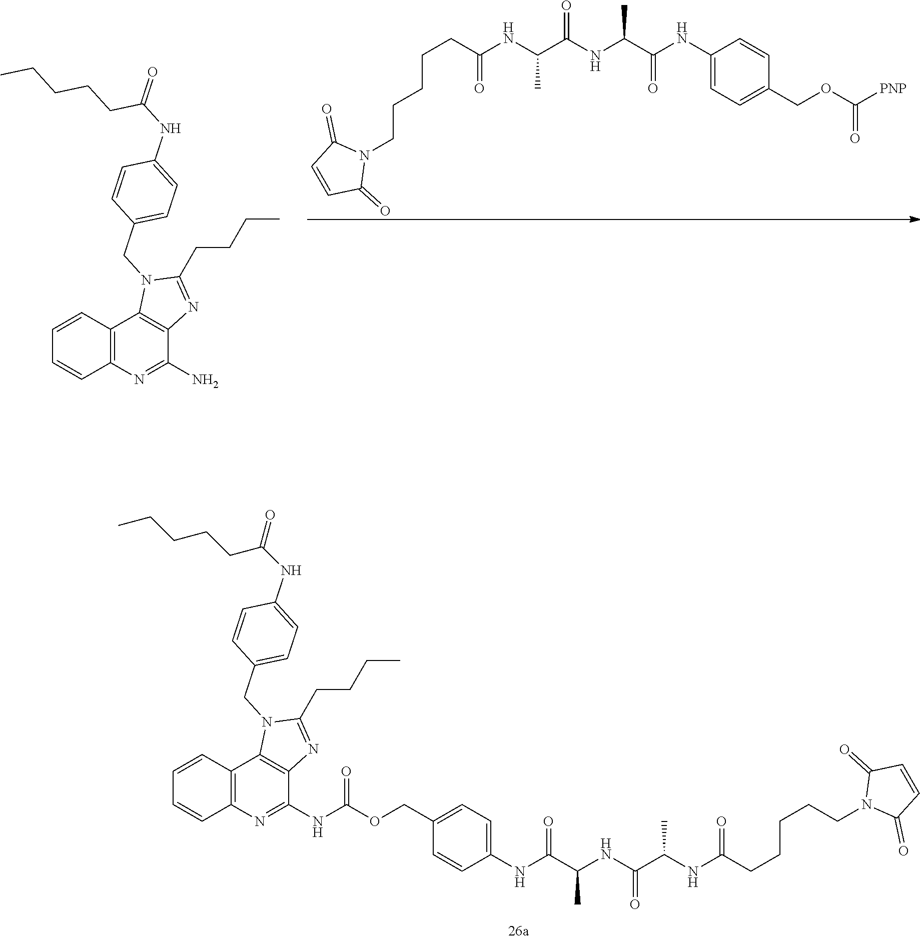

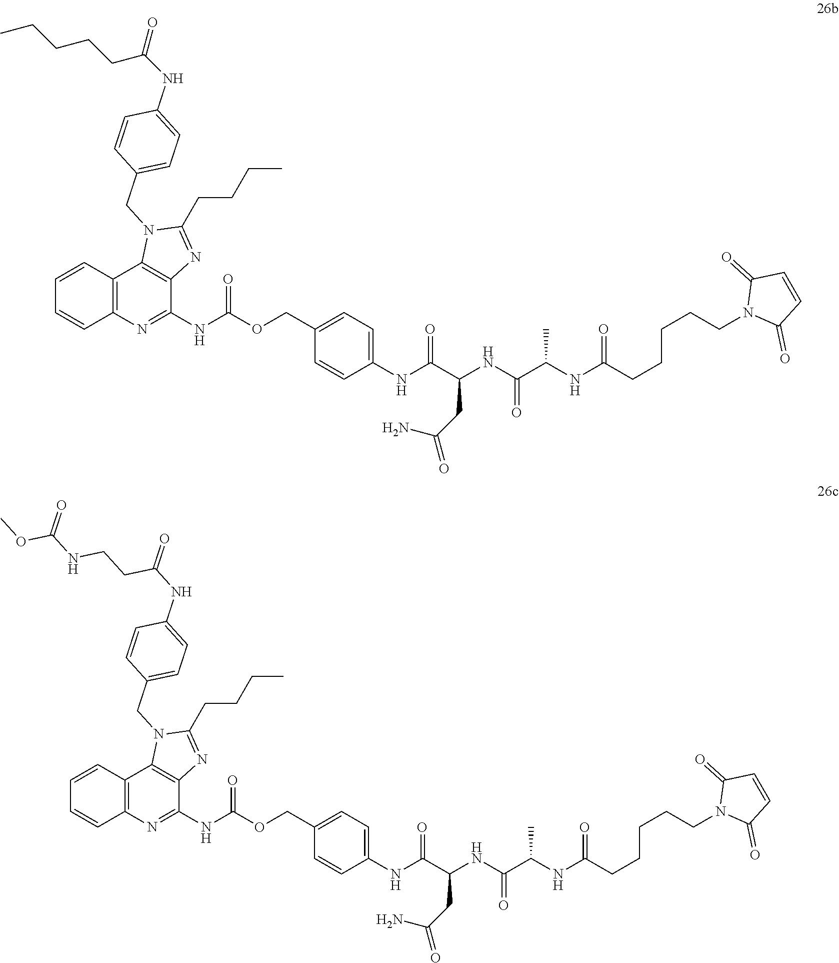 [SMILES: CCCCCC(=O)Nc1ccc(Cn2c(CCCC)nc3c(NC(=O)OCc4ccc(NC(=O)[C@H](CC(N)=O)NC(=O)[C@H](C)NC(=O)CCCCCN5C(=O)C=CC5=O)cc4)nc4ccccc4c32)cc1 ; CCCCc1nc2c(NC(=O)OCc3ccc(NC(=O)[C@H](CC(N)=O)NC(=O)[C@H](C)NC(=O)CCCCCN4C(=O)C=CC4=O)cc3)nc3ccccc3c2n1Cc1ccc(NC(=O)CCNC(=O)OC)cc1]

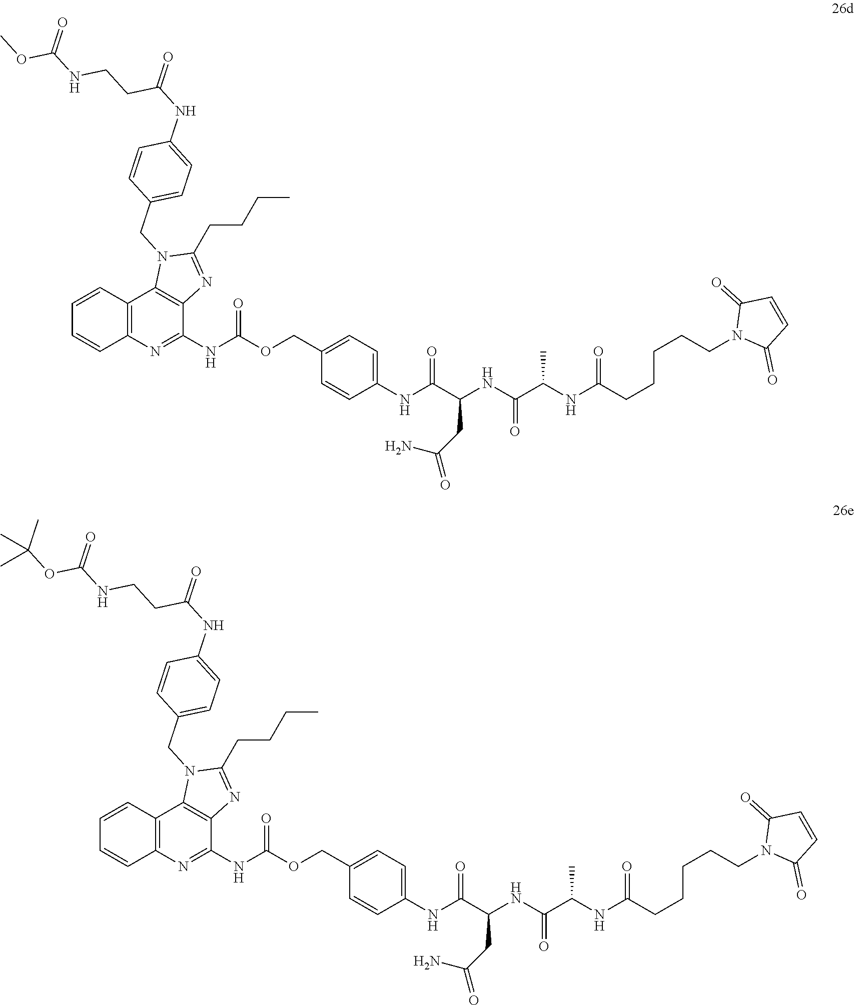 [SMILES: CCCCc1nc2c(NC(=O)OCc3ccc(NC(=O)[C@H](CC(N)=O)NC(=O)[C@H](C)NC(=O)CCCCCN4C(=O)C=CC4=O)cc3)nc3ccccc3c2n1Cc1ccc(NC(=O)CCNC(=O)OC)cc1 ; CCCCc1nc2c(NC(=O)OCc3ccc(NC(=O)[C@H](CC(N)=O)NC(=O)[C@H](C)NC(=O)CCCCCN4C(=O)C=CC4=O)cc3)nc3ccccc3c2n1Cc1ccc(NC(=O)CCNC(=O)OC(C)(C)C)cc1]

4-((S)-2-((S)-2-(6-(2,5-dioxo-2,5-dihydro-1H-pyrrol-1-yl)hexanamido)propanamido)propanamido)benzyl (2-butyl-1-(4-hexanamidobenzyl)-1H-imidazo[4,5-c]quinolin-4-yl)carbamate: The agonist (N-(4-((4-amino-2-butyl-1H-imidazo[4,5-c]quinolin-1-yl)methyl)phenyl)hexanamide) is dissolved in DMA and treated subsequently with 2,6-lutidine (2 eq), HOBt (1 eq), and mcValCitPAB-PNP. After stirring overnight at rt, the desired product is isolated by preparative HPLC. The additional products shown (26a, 26b, 26c, 26d, 26e) may be prepared through minor modifications of this methodology.

### Example 27: Demonstration of Linker Attachment Site

LCMS/MS fragmentation analysis of various linker-payloads was performed into order to establish the regiochemistry of linker addition. One such example is illustrated below. The structure obtained (which was assumed by mass to be either isomer I or isomer II) was fragmented using a Waters TQD LCMS system under “daughter ion scan” mode. Fragment III (m/z=241) was prominently observed, while fragment IV (m/z=548) was not observed. Based on this, it was concluded that the parent structure consisted of compound I. NMR analysis supported this structural determination.

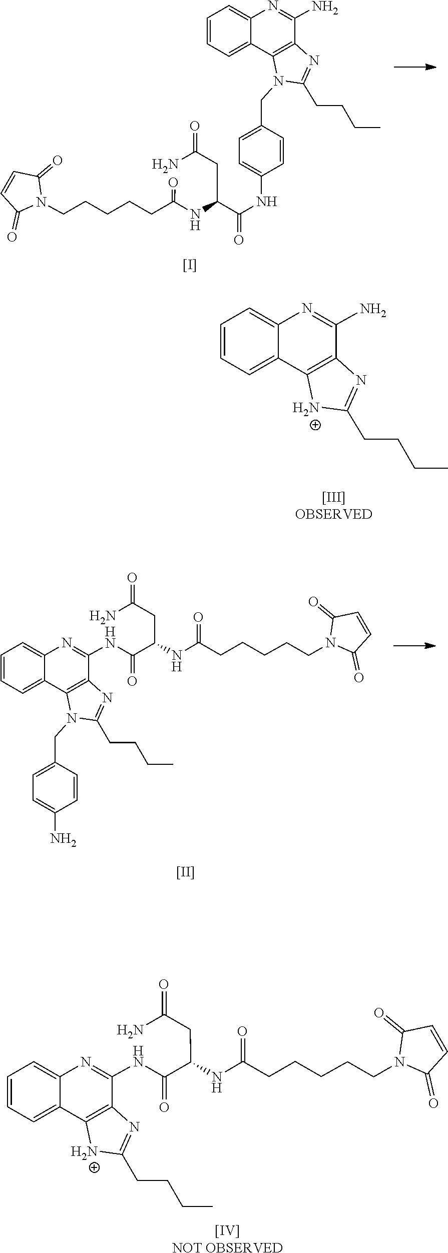

### Example 28: Preparation of New ADCs Using a Site-Specific Thiolation to Attach Payloads to the Q295 Residue

Deglycosylation: 1 mg of an IgG1 antibody (as shown below) was treated with 4 μl (2 ug) of PNGase F (Bulldog Bio) and diluted to 500 ul with PBS. The reaction was incubated at 37° C. overnight or until deglycosylation is complete as determined by LCMS. Thiolation: After complete deglycosylation was observed, 240 uL of 0.1 M Sorensen's phosphate buffer (pH 6) was added followed by 20 uL of 30 mM aqueous cystamine (100 eq.) and 70 mg of transglutaminase powder (Ajinomoto). The mixture was vortexed thoroughly, gently heating until the solution became homogeneous. Protein A capture: After incubating at 37° C. for 48 h, the material was added to a protein A column that had been pre-equilibrated in PBS. Selective reduction of disulfide: The resin was washed 3×600 uL with PBS and then treated with 100 uL of PBS containing 10 eq of TPPMS (aka sodium diphenylphosphinobenzene-3-sulfonate). After incubation for 2 h at rt, the resin was centrifuged to remove the excess TPPMS. Conjugation with the appropriate LP: The resin was treated with 100 uL of PBS containing 12 eq of the appropriate linker-payload. (Note that the linker-payload was stored as a 10 mM stock solution in DMA.) After briefly vortexing, the resin was allowed to sit at RT overnight. [Note that in some cases additional DMSO was added in order to prevent precipitation of the linker-payload.] Elution: The resin was washed 2× with 600 uL of PBS and then treated with 400 ul of glycine buffer (pH 4). After standing for 3 mins, the resin was centrifuged into a tube containing 30 ul of Tris buffer (pH 7.8). The elution step was repeated again to ensure complete elution of the ADC. The combined eluants were buffer exchanged into 1 mL of PBS and filter sterilized for storage.

TABLE
ADCs prepared by the above method:

| | | Final | Mass | Theoretical | % |
|---|---|---|---|---|---|
| Antibody | Linker-payload | DAR | shift | mass shift | Aggregation |
| Anti-Her2 | mcE104 (LP#11) | 1.6 | 535 | 537 | NA |
| Anti-Her2 | mcValCitPABC-E104 (LP#9) | 2.0 | 946 | 944 | NA |
| Anti-GCC | mcE104 (LP#11) | 1.6 | 539 | 537 | NA |
| Anti-GCC | mcValCitPABC-E104 (LP#9) | 2.0 | 946 | 944 | NA |
| Anti-Trop2 | mcE104 (LP#11) | 1.7 | 543 | 537 | NA |
| Anti-Trop2 | mcValCitPABC-E104 (LP#9) | 2.0 | 943 | 944 | NA |
| Anti-RSV | mcE104 (LP#11) | 1.7 | 541 | 537 | NA |
| Anti-RSV | mcValCitPABC-E104 (LP#9) | 2.1 | 946 | 944 | NA |

### Example 29: Demonstration of Efficacy in a Mouse Xenograft Model

A breast cancer xenograft study was performed in SCI/beige mice in order to establish the utility and targeting ability of the ADCs. Human breast cancer cells (HCC1954, ATCC) were cultured in Roswell Park Memorial Institute (RPMI) 1640 (Corning) supplied with 10% FBS (Avantar) and maintained under the manufacturer's recommended densities. 100 U ml-1 penicillin-streptomycin was applied to prevent microbe contamination. Before the tumor implantation, the cells were trypsinized, rinsed, and re-suspended into a suspension of 40 million cells ml-1. Immediately before the implantation, the suspension was mixed 1:1 with Matrigel (Corning) to form the final implantation mixture, which was kept on ice for no longer than 2 hours. Approximately 2 million cells (100 μl of the mixture) was implanted subcutaneously to the right flank of 6-8 weeks old female SCID/beige mice (Charles River Labs). Tumor volume was recorded twice a week using a caliper and estimated using the following formula: length×width²/2.

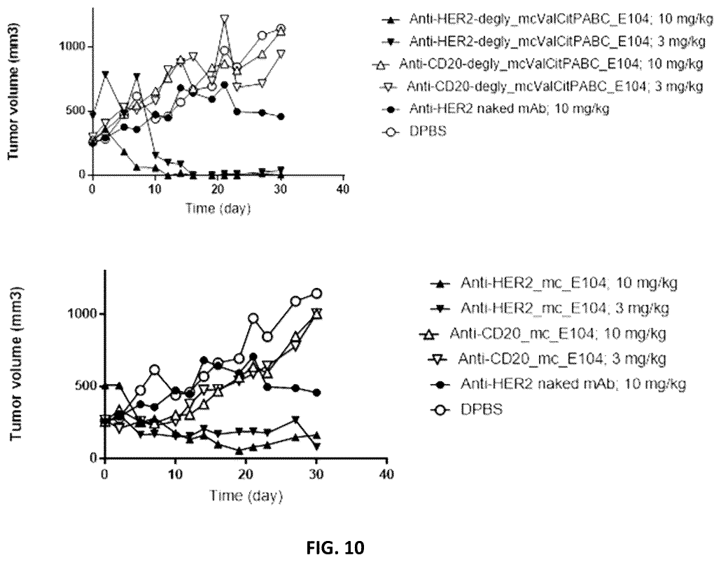

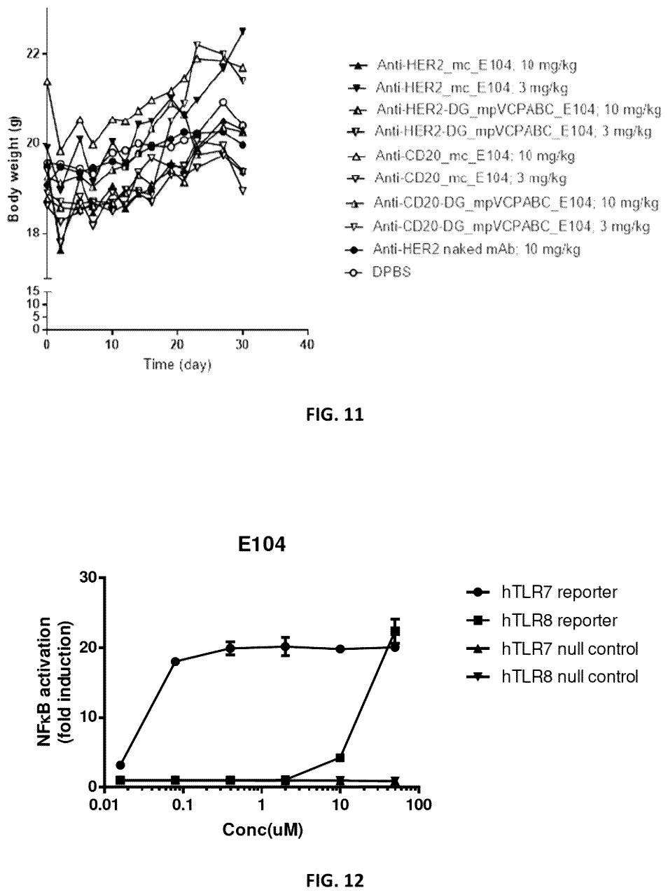

Treatment was initiated once the tumor volumes reached 50-300 mm³. Mice were randomly assigned to 10 different treatment groups (5 mice per group). The mice were dosed with ADCs (10 mg kg⁻¹ or 3 mg kg⁻¹), naked anti-Her2 mAb (10 mg kg⁻¹), or DPBS via intraperitoneal injection 3 times in total with 5-day intervals. Tumor volumes were measured and recorded every 2-3 days. Mice whose tumor exceeded 1000 mm³, suffered from ulceration, or displayed any signs of stress during the study were euthanized based on IACUC approved animal protocols. The results are shown in FIG. 10. In short, treatment with the targeted (anti-Her2) ADCs resulted in rapid tumor regression while treatment with the corresponding non-targeted (anti-CD20) ADCs did not. No significant changes in body weight were observed for any of the treatment groups, suggesting that the ADCs were well tolerated. (FIG. 11)

### Example 30: Evaluation of Payloads in HEK-Blue mTLR7, hTLR7, mTLR8, and Htlr8 Cells

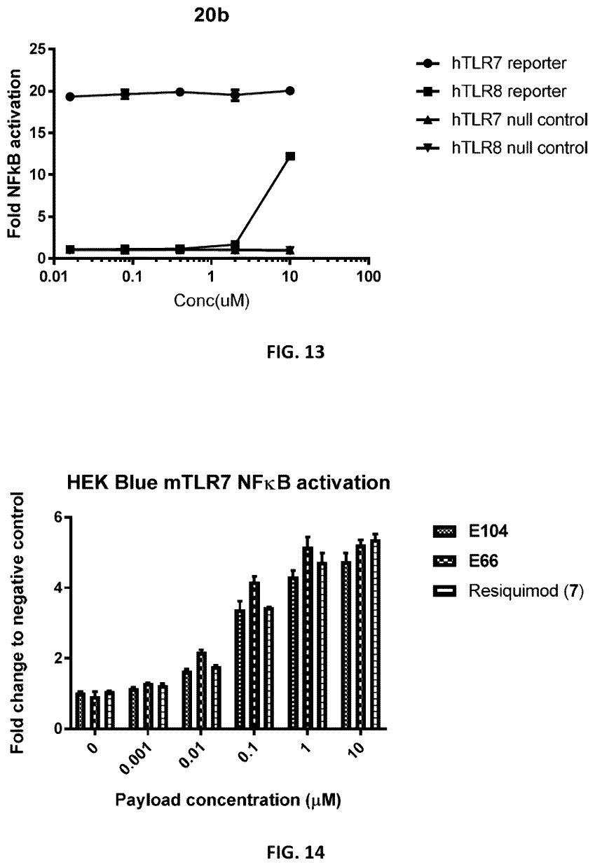

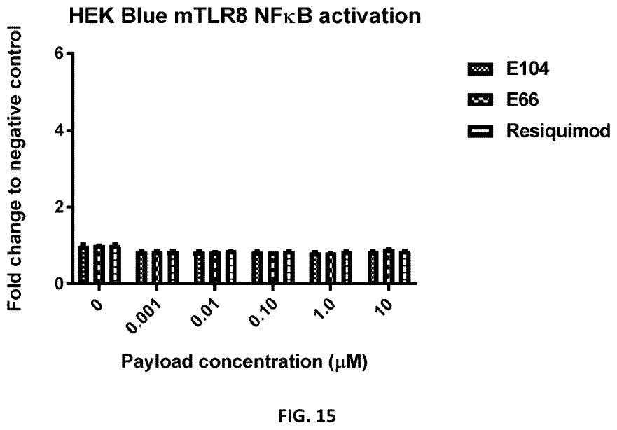

HEK reporter cell lines (Invivogen) were maintained in culture media using high glucose DMEM media supplied with 10% FBS, supplemented with 50 U/mL penicillin, 50 ug/mL streptomycin, and 100 ug/mL normocin to prevent bacterial contamination. Prior to the experiment, cells were rinsed and detached using prewarmed DPBS and collected by centrifugation at 1100 rpm for 5 min. Cells were then re-suspended at 0.2 million cells per mL and seeded into 96 well plates (90 uL). Compounds were diluted to 10× the final desired concentrations in PBS. 10 μL of the payload solution (or PBS blank) was added to each well. Each experiment was performed in triplicate. The plate was incubated at 37° C./5% CO₂ environment for 24 h. The supernatants were collected, and the SEAP induction (NFκB activation) was determined using a QUANTI-Blue™ substrate, per the manufacturer's protocol, by measuring the absorbance at 630 nm or 650 nm. Results shown in FIGS. 12-15 indicate that select compounds of the invention are potent and selective TLR7 agonists against both human and mouse isotypes, while weak to modest TLR8 activity is observed.

### Example 31: Evaluation of NFkB Activation in Ramos-Blue Cells for Additional TLR7 Agonists

A 3× serial dilution was performed in 10% DMSO in PBS for each payload to have a final range of concentration from 1000 uM to 76 nM. Ramos-blue cells (InvivoGen, cat #rms-sp) were cultured using high glucose DMEM media supplied with 10% fetal bovine serum according to the manufacturer guidelines. The media was supplemented with 50 U/mL penicillin, 50 ug/mL streptomycin, and 100 ug/mL normocin to prevent bacterial contamination. The cell density and viability were calculated using a Countess Cell Counter and the proper volume of cells was removed in order to have a seeding density of 0.2×10⁶ cells/mL per well. 135 uL of the cell suspension was added to each well in a 96-well plate along with 15 uL of the corresponding payload treatment. Each assay point was run in triplicate and the plate was incubated at 37° C. with 5% CO₂ for 24 or 72 hours. In order to assess the NFκB induction, the QUANTI-Blue™ solution was prepared by adding 200 uL of QB reagent (InVivogen cat #rep-qbs) and 200 uL of QB buffer to 19.6 mL of water. The resulting solution was vortexed and incubated at room temperature for ten minutes. The 96-well plate was centrifuged at 1990 rpm for ten minutes and 40 uL of the cell supernatant was added to 160 uL of the prepared QUANTI-Blue™ solution. The QB reaction plate was incubated at 37° C. for 24 hours. The plate was read using the Molecular Devices i3x plate reader at a wavelength of 630 nm to determine the amount of SEAP production. The table below illustrates the lowest concentration of each compound that results in a doubling of the SEAP background (nontreated) signal. As illustrated, compounds of the invention ranged from low nM to low micromolar.

| | | Fold increase in |
|---|---|---|
| | | SEAP signal at |
| | Lowest concentration | lowest active |
| Compound ID | to induce 2x SEAP | concentration |

| | | | |
|---|---|---|---|
| E104 | 23 | nM | 2.327088212 |
| 21e | 620 | nM | 5.753256151 |
| 21f | 210 | nM | 2.901750973 |
| 22e | 210 | nM | 2.537285173 |
| 23e | 1,870 | nM | 4.344328965 |
| 20a | 7.6 | nM | 3.011959522 |
| 20f | 69 | nM | 2.225444341 |
| 22f | 69 | nM | 2.269152345 |
| 20a | 23 | nM | 3.22655218 |
| 20b | 7.6 | nM | 4.415904071 |
| 20c | 7.6 | nM | 2.351869919 |
| 20d | 7.6 | nM | 2.221555089 |
| 20e | 69 | nM | 2.98338558 |
| E104 | 23 | nM | 2.037505972 |
| 20j | 210 | nM | 2.80248307 |
| 20k | 7.6 | nM | 2.397791353 |
| 20h | 620 | nM | 3.232208486 |
| 20l | 23 | nM | 2.110220441 |
| 23f | 23 | nM | 1.946024636 |
| E104 | 69 | nM | 4.853590302 |
| 20m | 23 | nM | 3.188190717 |
| 20n | 7.6 | nM | 1.925180761 |
| 20o | 69 | nM | 4.1578125 |
| 20g | 620 | nM | 2.637920489 |
| 20h | 210 | nM | 2.085663821 |

NA = Not active

### Example 32. Delivery of TLR Agonists to Pancreatic Cancer Cell Line BXPC3 and Non-Small Cell Lung Cancer Cell Line A549 Using Anti-Trop2 and Anti-GCC Antibodies

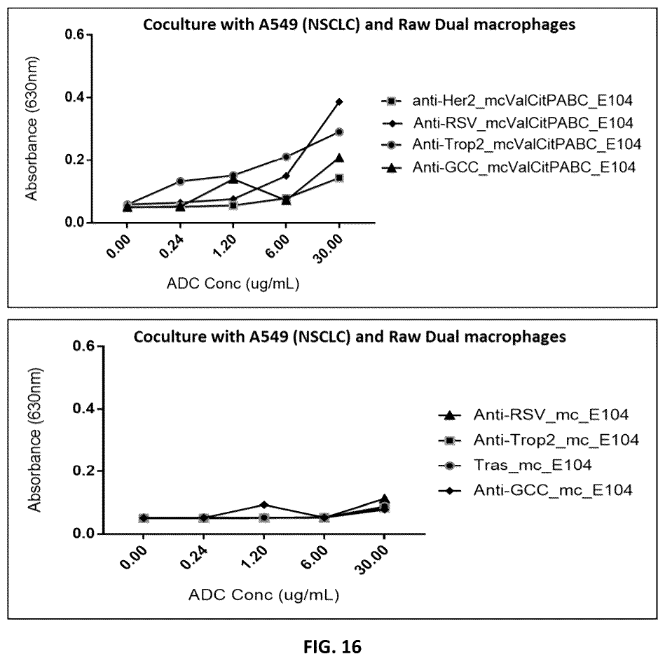

A co-culture experiment was performed wherein 5000 cells each of A549 (non-small cell lung cancer) and mouse macrophage Raw Dual (Invivogen) were cultured at 37° C. under a 5% CO2 atmosphere in DMEM high glucose/10% FBS+Pen/Strep. The cells were cultured for 48 h in the presence of various concentrations of select ADCs. A 20 uL aliquot of the media was removed and added to 180 uL of QUANTI-Blue™ solution (Invivogen). After incubation for 4 h or 24 h, the absorbance at 630 nM indicated the activation of the NFκB pathway in the macrophage cell line. The results are shown in FIG. 16. The results provide evidence that anti-Trop2 and anti-GCC ADCs of the present invention are capable of simulating macrophages in the vicinity of antigen-expressing non-small cell lung cancer tissue.

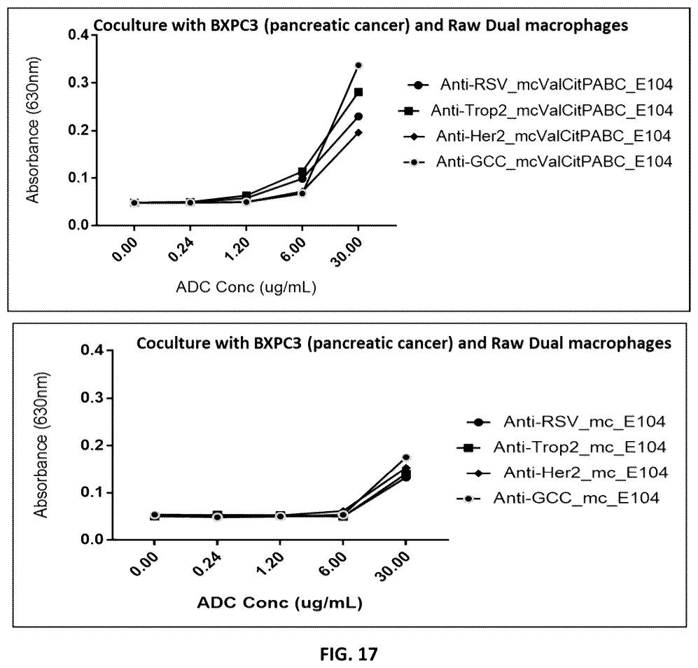

An analogous experiment was performed with BXPC3 cells (pancreatic cancer), likewise co-cultured with the mouse macrophage cell line Raw Dual. The results are shown in FIG. 17. The results provide evidence that anti-Trop2 and anti-GCC ADCs of the present invention are capable of simulating macrophages in the vicinity of antigen-expressing pancreatic cancer tissue.

### Example 33. TLR-Activating ADCs are Nontoxic to Her2 Expressing Breast Cancer Cells

SKBR3 and HCC1954 cells were cultured in RPMI1640 media supplied with 10% fetal bovine serum before the assay. In brief, SKBR3 and HCC1954 cells were harvested and resuspended into seeding suspensions of 0.2 million cells per mL. Then 90 μL of the suspension was seeded into 96 well plates. The ADCs (anti-Her2_mcE104 and anti-Her_mcValCitPABC_E104) were diluted into concentration gradients (3 fold serial dilution, 10 different concentrations including 0), and 10 μL of ADC solutions were added to corresponding wells and mixed gently. The plates were then incubated under 37° C./5% CO₂ for 72 hours. After the incubation, cell viabilities were obtained using XTT Cell Viability Assay Kit (Biotium). IC50 curves were generated in Graphpad Prism software using “log[inhibitor] vs response—Variable slope (four parameters)” equation. The IC50 of both ADCs was shown to be >30 g/mL against both HCC1954 and SKBR3 cells. This data indicates that the in vivo efficacy observed (Example 29) is not due to direct toxicity, but rather to indirect activation of nearby tumor-associated lymphocytes.

Various preferred embodiments [A] to [AQ] of the invention can be described in the text below:

[Embodiment A]A compound of the Formula (I) or (II)

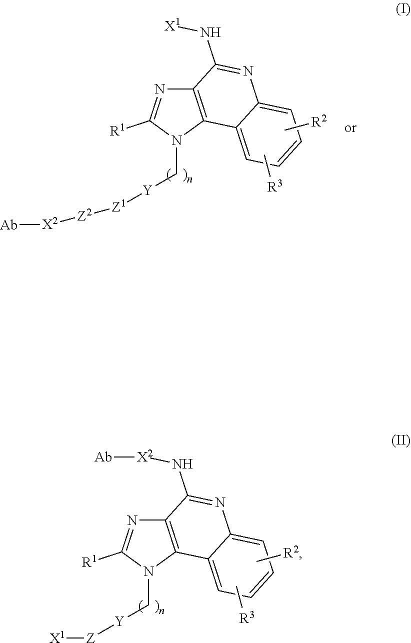

wherein:
   R¹ is selected from C₁-C₁₀ alkyl, C₁-C₁₀ oxaalkyl, and C₁-C₁₀ azaalkyl;
   R² and R³ are each independently selected from hydrogen, C₁-C₅ alkyl, and C₁-C₅ alkoxy;
   n is 1 or 2
   Y is independently selected from optionally substituted aryl and optionally substituted heteroaryl;
   Z¹ is selected from —NR^{Z}—, —O—, —NR^{Z}C(O)—, —NR^{Z}C(O)—O—, and —NR^{Z}SO₂—;
   Z² is absent, or is selected from (C₁-C₈)hydrocarbon-NH— and a 5- to 8-membered nitrogen-containing heterocycle, wherein a nitrogen of the heterocycle is attached to X²;
   Z is independently selected from —NR^{Z}—, —NR^{Z}C(O)—, and —O—;
   R^{Z} is independently selected in each instance from hydrogen, C₁-C₈ hydrocarbon, C₁-C₈ oxaalkyl, C₁-C₈ azaalkyl, heteroaryl, and a 5- to 8-membered heterocyclic ring;
   X¹ is independently selected from R^{Z}, —C(O)—R^{Z}, —C(O)—O—R^{Z}, —C(O)—N—(R^{Z})₂, —(CH₂)ₖNR^{Z}C(O)—(C₁-C₆)alkyl, —(CH₂)ₖNR^{Z}C(O)—O—(C₁-C₄)alkyl, and —SO₂—R^{Z};
   k is an integer from 1 to 8;
   X² comprises cleavable or noncleavable linker; and
   Ab comprises an antibody or an antibody fragment.

[Embodiment B]A compound of Embodiment [A] above, or according to other embodiments of the invention, of the Formula (I) or (II)

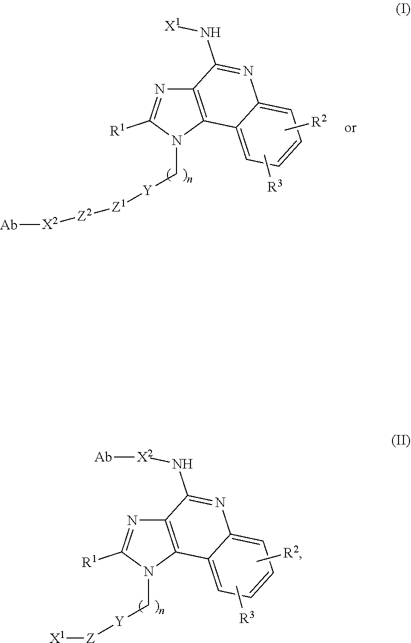

wherein:
   R¹ is selected from C₁-C₁₀ alkyl, C₁-C₁₀ oxaalkyl, and C₁-C₁₀ azaalkyl;
   R² and R³ are each independently selected from hydrogen, C₁-C₅ alkyl, and C₁-C₅ alkoxy;
   n is 1 or 2
   Y is independently selected from optionally substituted aryl and optionally substituted heteroaryl;
   Z¹ is selected from —NR^{Z}—, —O—, —NR^{Z}C(O)—, —NR^{Z}C(O)—O—, and —NR^{Z}SO₂—;
   Z² is absent, or is selected from (C₁-C₈)hydrocarbon-NH— and a 5- to 8-membered nitrogen-containing heterocycle, wherein a nitrogen of the heterocycle is attached to X²;
   Z is selected from —NR^{Z}— and —O—;
   R^{Z} is independently selected in each instance from hydrogen, C₁-C₈ hydrocarbon, C₁-C₈ oxaalkyl, C₁-C₈ azaalkyl, heteroaryl, and a 5- to 8-membered heterocyclic ring;
   X¹ is independently selected from R^{Z}, —C(O)—R^{Z}, —C(O)—O—R^{Z}, —C(O)—N—(R^{Z})₂, and —SO₂—R^{Z};
   X² comprises cleavable or noncleavable linker; and
   Ab comprises an antibody or an antibody fragment.

[Embodiment C] A compound of any one of Embodiments [A] or [B] above, or according to other embodiments of the invention, wherein Z² is absent, or is selected from —(C₁-C₈)alkyl-NH—, -benzyl-NH—, phenyl-NH, and a 5- to 8-membered nitrogen-containing heterocycle.

[Embodiment D] A compound of any one of Embodiments [A] to [C] above, or according to other embodiments of the invention, wherein:
X² is L1-L2-(L3)ₚ-(L4)_{q}-(L5)ᵣ;
L1 is a conjugation moiety;
L2 is a spacer unit selected from branched or unbranched C₁-C₁₂ alkyl, a PEG selected from PEG1 to PEG12,

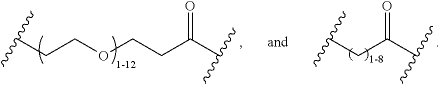

L3 is a peptide of 1 to 6 amino acids;
L4 is a self-immolative spacer;
L5 is carbonyl; and
p, q, and r are each independently selected from 0 and 1, wherein when p and q are each 0, r must be 0.

[Embodiment E] A compound of any one of Embodiments [A] to [D] above, or according to other embodiments of the invention, wherein the compound is of Formula (I), and:
R¹ is selected from n-butyl, —CH₂OH, and —CH₂OCH₂CH₃;
R² and R³ are each hydrogen;
n is 1;
Y is phenyl or pyridyl, each of which is unsubstituted or substituted with one or more of halogen, C₁-C₄ alkyl, C₁-C₄ alkoxy, C₁-C₄ haloalkyl, or C₁-C₄ haloalkoxy;
X¹ is hydrogen; and
Z¹ is —N(R^{Z})— or —O—.

[Embodiment F] A compound of any one of Embodiments [A] to [D] above, or according to other embodiments of the invention, wherein the compound is of Formula (II), and:
R¹ is selected from n-butyl, —CH₂OH, and —CH₂OCH₂CH₃;
R² and R³ are each hydrogen;
n is 1;
Y is phenyl or pyridyl, each of which is unsubstituted or substituted with one or more of halogen, C₁-C₄ alkyl, C₁-C₄ alkoxy, C₁-C₄ haloalkyl, or C₁-C₄ haloalkoxy;
X¹ is hydrogen; and
Z is —N(R^{Z})— or —O—.

[Embodiment G] A compound of any one of Embodiments [A] or [B] above, or according to other embodiments of the invention, wherein the compound of Formula (I) is the compound of formulae (Ia), (Ib), (Ic), (Id), (Ie), (If), (Ig), or (Ih):

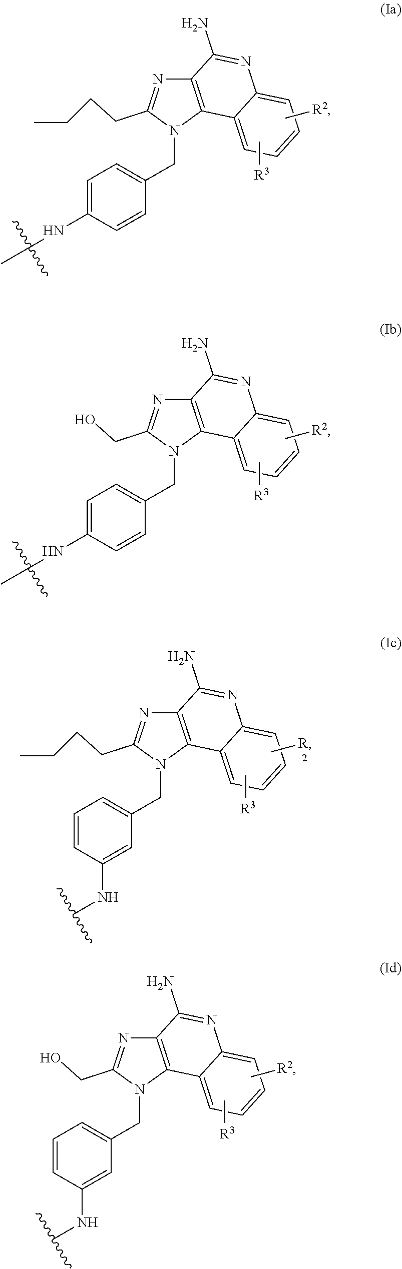

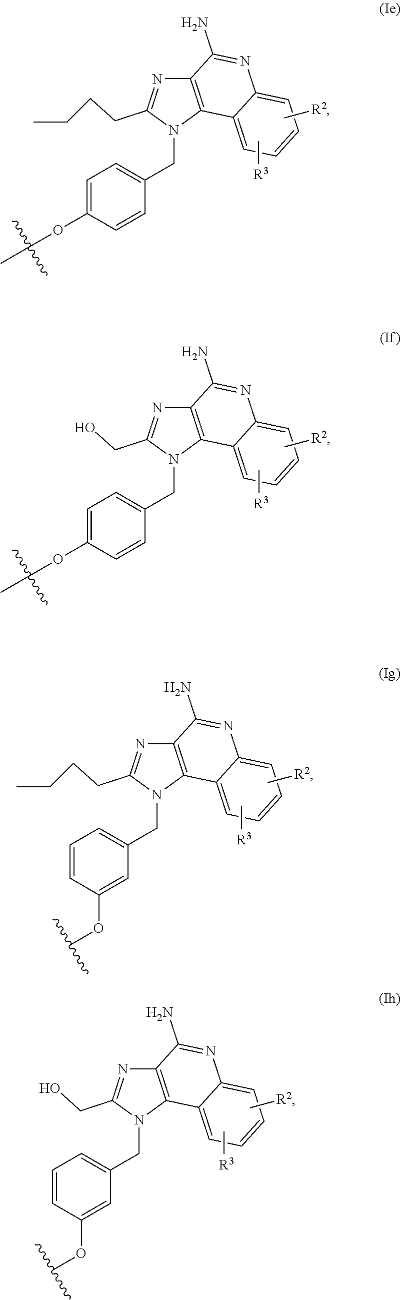

wherein [symbol] represents a point of attachment to X₂.

[Embodiment H] A compound of any one of Embodiments [A], [B] or [G] above, or according to other embodiments of the invention, wherein the compound of Formula (I) is the compound of formulae (Ia), (Ic), (Ie), or (Ig).

[Embodiment I] A compound of any one of Embodiments [A], [B], [G], or [H] above, or according to other embodiments of the invention, wherein the compound of Formula (I) is the compound of formulae (Ia).

[Embodiment J] A compound of any one of Embodiments [A] or [B] above, or according to other embodiments of the invention, wherein the compound of Formula (II) is the compound of formulae (IIa), (IIb), (IIc), (IId), (IIe), (IIf), (IIg), or (IIh):

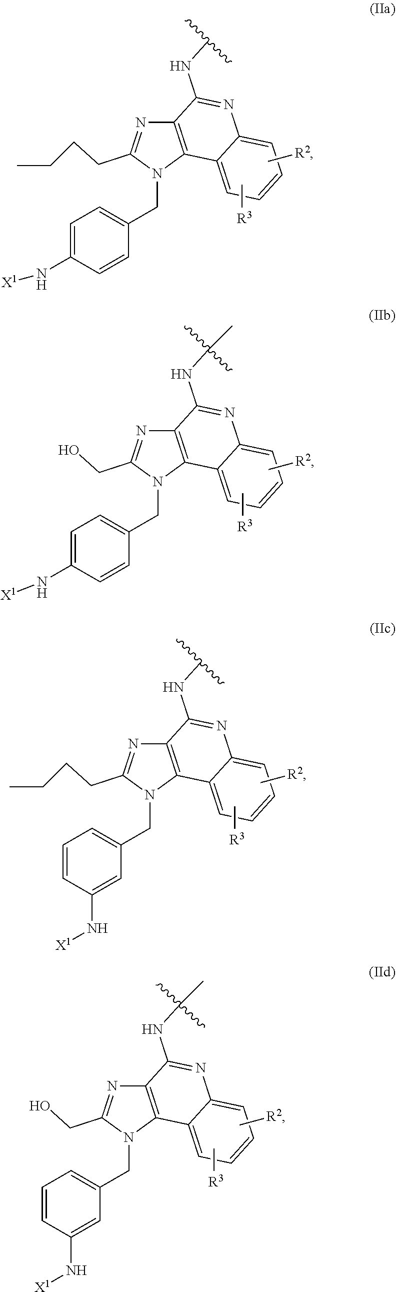

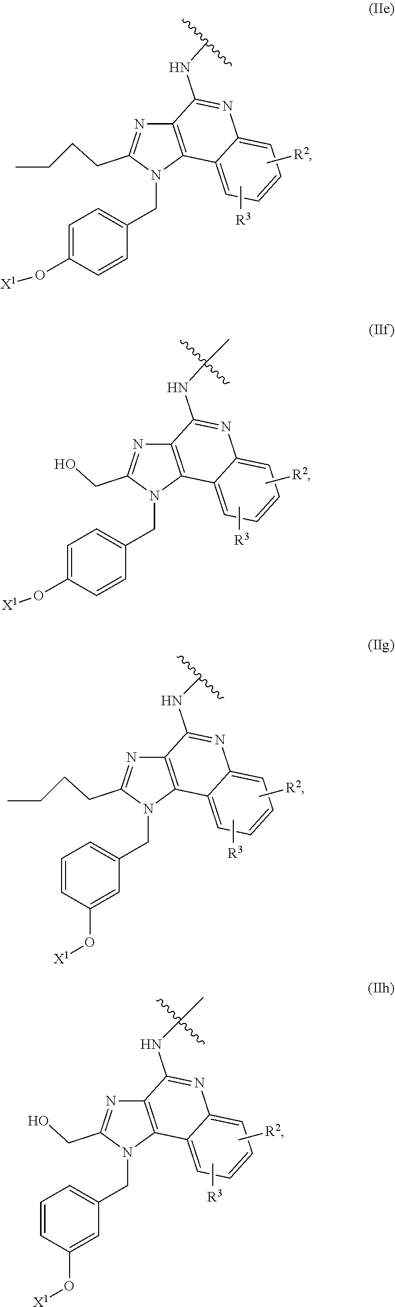

wherein [symbol] represents a point of attachment to X₂.

[Embodiment K] A compound of any one of Embodiments [A], [B] or [J] above, or according to other embodiments of the invention, wherein the compound of Formula (II) is the compound of formulae (IIa).

[Embodiment L] A compound of any one of Embodiments [A] to [K] above, or according to other embodiments of the invention, wherein X² is L1-L2-(L3)ₚ-(L4)_{q}-(L5)ᵣ; and
L1 is selected from:

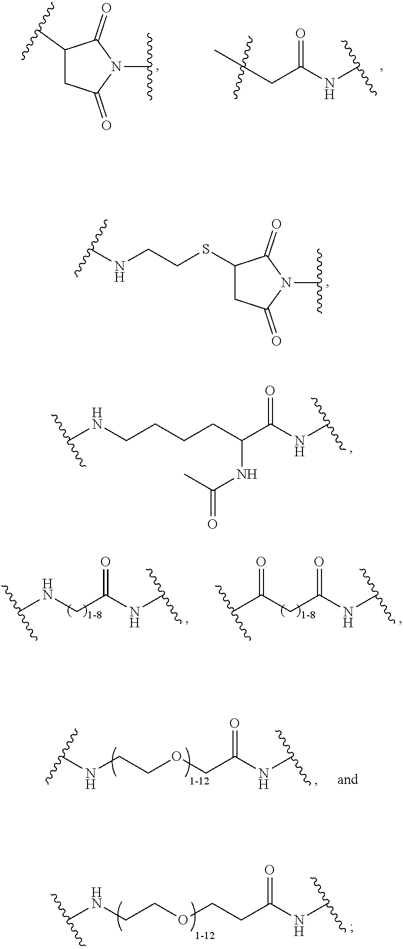

L2 is selected from

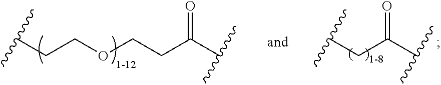

L3 is

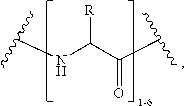

wherein R is an amino acid side chain;
L4 is selected from:

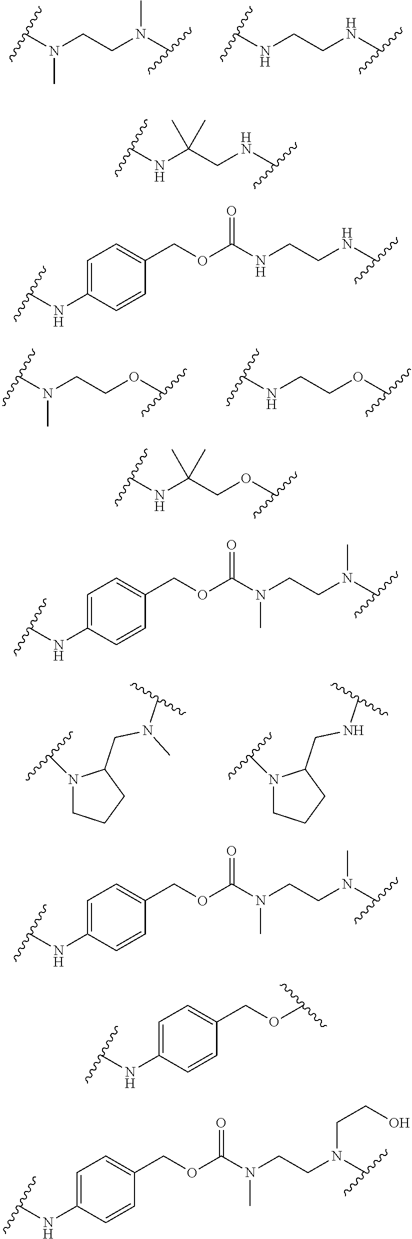

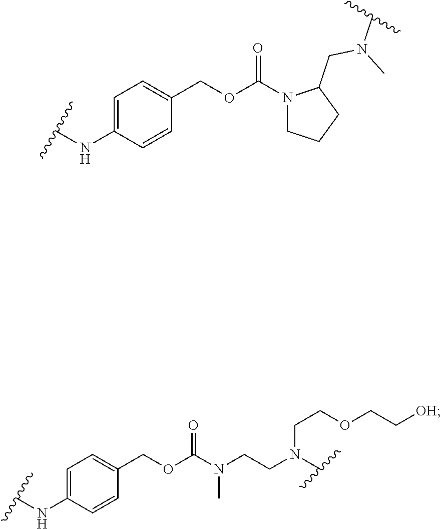 [SMILES: *Nc1ccc(COC(=O)N2CCCC2CN(*)C)cc1 ; *Nc1ccc(COC(=O)N(C)CCN(*)CCOCCO)cc1]

L5 is

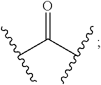 [SMILES: *C(*)=O]

and
p, q, and r are each independently 0 or 1, wherein when p and q are each 0, r must be 0.

[Embodiment M] A compound of Embodiment [L] above, or according to other embodiments of the invention, wherein L3 is selected from ValCit, GlyValCit, ValArg, PheLys, AlaAla, GlyGlyPheGly, AlaAlaAla, AlaAsn, AsnAsn, AsnAla, ValCitGlyPro, AsnGlyPro, AsnAsnGlyPro, Asn, GlyAsn, AsnAla, ProCitAla, ProAsnLeu, ProAsnAla, ProPheAla, ProPheGly, ProCitLeu, ProAsnPro, ProAsnSer, and ProAsnGly.

[Embodiment N] A compound of Embodiment [L] above, or according to other embodiments of the invention, wherein:
L1 is selected from

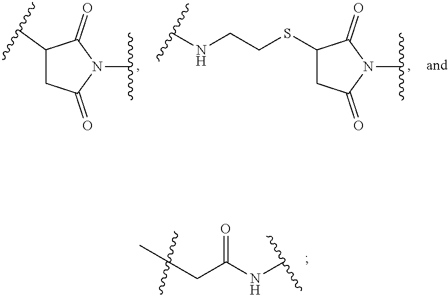 [SMILES: *C1CC(=O)N(*)C1=O ; *NCCSC1CC(=O)N(*)C1=O ; *CC(=O)N*]

L2 is

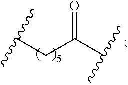

L3 is ValCit, GlyValCit, AsnAsn, Asn or AlaAla;
L4 is selected from:

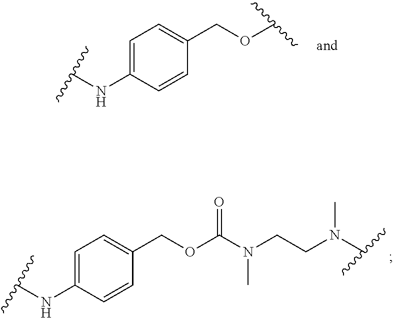 [SMILES: *Nc1ccc(CO*)cc1 ; *Nc1ccc(COC(=O)N(C)CCN(*)C)cc1]

L5 is

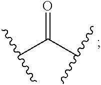 [SMILES: *C(*)=O]

and
p, q, and r are each 0 or p, q, an r are each 1.

[Embodiment O] A compound of any one of Embodiments [A] to [N] above, or according to other embodiments of the invention, wherein X² is attached to Ab through a cysteine residue of Ab, a lysine residue of Ab, or a glutamine residue of Ab, optionally glutamine 295.

[Embodiment P] A compound of any one of Embodiments [A] to [O] above, or according to other embodiments of the invention, wherein Ab is a tumor targeting antibody, an antibody fragment, a bispecific antibody or antibody fragment, a monoclonal antibody, a chimeric antibody, or a humanized antibody.

[Embodiment Q] A compound of any one of Embodiments [A] to [P] above, or according to other embodiments of the invention, wherein Ab is selected from the group consisting of anti-Her2 antibody, anti-CD20 antibody, anti-CD38 antibody, anti-IL-6 receptor antibody, anti-VEGRF2 antibody, anti-HER-2 antibody, anti-DLL3 antibody, anti-Nectin4 antibody, anti-CD33 antibody, anti-CD79b antibody, anti-CD11a antibody, anti-BCMA antibody, anti-CD22 antibody, anti-Trop2 antibody, anti-FRα antibody, anti-EpCAM antibody, anti-mesothelin antibody, anti-LIV1 antibody, oregovomab, edrecolomab, cetuximab, a humanized monoclonal antibody to the vitronectin receptor (αᵥβ₃), alemtuzumab, a humanized anti-HLA-DR antibody for the treatment of non-Hodgkin's lymphoma, 131l Lym-1, a murine anti-HLA-Dr10 antibody for the treatment of non-Hodgkin's lymphoma, a humanized anti-CD2 mAb for the treatment of Hodgkin's Disease or non-Hodgkin's lymphoma, labetuzumab, bevacizumab, ibritumomab tiuxetan, ofatumumab, panitumumab, rituximab, tositumomab, ipilimumab, gemtuzumab, humanized monoclonal antibody to the oncofecal protein receptor 5T4, M1/70 (antibody to CD11b receptor), anti-MRC1, anti GCC, anti CD32, and other antibodies.

[Embodiment R] A compound of the Formula (III)

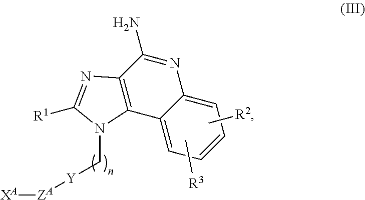

wherein:
R¹ is selected from C₁-C₁₀ alkyl, C₁-C₁₀ oxaalkyl, and C₁-C₁₀ azaalkyl;
R² and R³ are each independently selected from hydrogen, C₁-C₅ alkyl, and C₁-C₅ alkoxy;
n is 1 or 2;
Y is selected from optionally substituted aryl and optionally substituted heteroaryl;
Z^{A} is selected from —NR^{Z}—, —NR^{Z}C(O)—, —NR^{Z}C(O)—O—, —NR^{Z}C(O)—(CH₂)ₖ—NH—, —NR^{Z}C(O)—(CH₂)ₖ—O—, —C(O)—O—(CH₂)ₖ—O—, —NR^{Z}C(O)—(CH₂)ₖ—N(CH₃)—, —NR^{Z}C(O)—O—(CH₂)ₖ—NH—, —NR^{Z}C(O)—(CH₂)ₖ—NH—C(O)—O—, and —NR^{Z}SO₂—;
k is an integer from 1 to 8;
R^{Z} is selected from hydrogen, C₁-C₈ hydrocarbon, C₁-C₈ oxaalkyl, C₁-C₈ azaalkyl, heteroaryl, and a 5- to 8-membered heterocyclic ring; and
X^{A} is selected from hydrogen, C₁-C₁₀ alkyl, and —C(O)CH₃,
wherein the following compound is excluded:

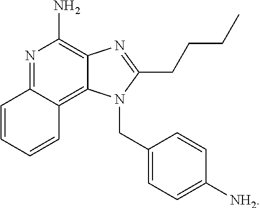 [SMILES: CCCCc1nc2c(N)nc3ccccc3c2n1Cc1ccc(N)cc1]

[Embodiment S] A compound of Embodiment [R] above, or according to other embodiments of the invention, wherein:
R¹ is selected from n-butyl, —CH₂OH, and —CH₂OCH₂CH₃;
R² and R³ are each hydrogen;
n is 1;
Y is phenyl or pyridyl, each of which is unsubstituted or substituted with one or more of halogen, C₁-C₄ alkyl, C₁-C₄ alkoxy, C₁-C₄ haloalkyl, or C₁-C₄ haloalkoxy; and
R^{Z}, when present, is hydrogen.

[Embodiment T] A compound of any one of Embodiments [R] or [S] above, or according to other embodiments of the invention, wherein R¹ is n-butyl and Y is unsubstituted phenyl.

[Embodiment U] A compound of any one of Embodiments [R] to [T] above, or according to other embodiments of the invention, wherein Z^{A} is selected from —NR^{Z}, —NR^{Z}C(O)—, —NR^{Z}C(O)—O—, —NR^{Z}C(O)—(CH₂)ₖ—NH—, —N C(O)—O—(CH₂)ₖ—NH—, —NR^{Z}C(O)—(CH₂)ₖ—NH—C(O)—O—, and —NR^{Z}SO₂—.

[Embodiment V] A compound of any one of Embodiments [R] to [T] above, or according to other embodiments of the invention, wherein Z^{A}—X^{A} is selected from —NHC(O)O(C₁-C₄)alkyl, —NH₂, —NHC(O)(CH₂)ₖNH₂, —NHC(O)(CH₂)ₖNH—C(O)O(C₁-C₄)alkyl, and —NHC(O)(C₁-C₄)alkyl.

[Embodiment W] A compound of any one of Embodiments [A] to [V] above, or according to other embodiments of the invention, wherein k is an integer from 1 to 4.

[Embodiment X] A compound of any one of Embodiments [A] to [V] above, or according to other embodiments of the invention, wherein k is an integer from 1 to 6, or k is an integer from 1 to 3, or k is an integer from 1 to 2, or k is an integer from 2 to 4, or k is an integer from 2 to 3.

[Embodiment Y] A pharmaceutical composition comprising the compound of any one of Embodiments [A] to [X] above, or according to other embodiments of the invention, and a pharmaceutically acceptable carrier, diluent, or excipient.

[Embodiment Z] A pharmaceutical composition of Embodiment [Y] above, or according to other embodiments of the invention, further comprising a therapeutically effective amount of a chemotherapeutic agent.

[Embodiment AA] A method for stimulating an immune response in a subject, the method comprising administering a therapeutically effective amount of the compound of any one of Embodiments [A] to [X] above, or according to other embodiments of the invention, under conditions effective to stimulate an immune response.

[Embodiment AB] A method of Embodiment [AA] above, or according to other embodiments of the invention, wherein the administering is performed on a subject having cancer.

[Embodiment AC] A method of any one of Embodiments [AA] or [AB] above, or according to other embodiments of the invention, wherein the cancer is bladder cancer, breast cancer, cervical cancer, colon cancer, endometrial cancer, kidney cancer, lung cancer, esophageal cancer, ovarian cancer, prostate cancer, pancreatic cancer, skin cancer, gastric cancer, testicular cancer, biliary cancer, colorectal cancer, endometrial cancer, head and neck cancer, medullary thyroid cancer, renal cancer, eye cancer, neuroblastoma, Mycosis fungoides, glial and other brain and spinal cord tumors, liver cancer, leukemias, lymphomas, or any combination thereof.

[Embodiment AD] A method for inducing an anti-tumor immune response in a subject, the method comprising administering a therapeutically effective amount of the compound of any one of Embodiments [A] to [X] above, or according to other embodiments of the invention, under conditions effective to induce an anti-tumor immune response.

[Embodiment AE] A method of Embodiment [AD] above, or according to other embodiments of the invention, wherein the administering is performed on a selected subject having a tumor.

[Embodiment AF] A method of any one of Embodiments [AD] or [AE] above, or according to other embodiments of the invention, wherein the tumor is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

[Embodiment AG] A method for treating a tumor or abnormal cell proliferation, the method comprising administering a therapeutically effective amount of the compound of any one of Embodiments [A] to [X] above, or according to other embodiments of the invention, under conditions effective to treat a tumor or abnormal cell proliferation.

[Embodiment AH] A method of Embodiment [AG] above, or according to other embodiments of the invention, wherein the tumor or abnormal cell proliferation is cancer.

[Embodiment AI] A method of any one of Embodiments [AG] or [AH] above, or according to other embodiments of the invention, wherein the cancer is bladder cancer, breast cancer, cervical cancer, colon cancer, endometrial cancer, kidney cancer, lung cancer, esophageal cancer, ovarian cancer, prostate cancer, pancreatic cancer, skin cancer, gastric cancer, testicular cancer, biliary cancer, colorectal cancer, endometrial cancer, head and neck cancer, medullary thyroid cancer, renal cancer, eye cancer, neuroblastoma, Mycosis fungoides, glial and other brain and spinal cord tumors, liver cancer, leukemias, lymphomas, or any combination thereof.

[Embodiment AJ] A method for treating an infectious disease, the method comprising administering a therapeutically effective amount of the compound of any one of Embodiments [A] to [X] above, or according to other embodiments of the invention, under conditions effective to treat an infectious disease.

[Embodiment AK] A method of Embodiment [AJ] above, or according to other embodiments of the invention, wherein the infectious disease is a viral infection, a bacterial infection, a fungal infection, or any combination thereof.

[Embodiment AL] A method of any one of Embodiments [AJ] or [AK] above, or according to other embodiments of the invention, wherein the infectious disease is a viral infection.

[Embodiment AM] A method of any one of Embodiments [AJ] to [AL] above, or according to other embodiments of the invention, wherein the infectious disease is selected from coronaviruses, Ebola, influenza, hepatitis, Hib disease, human immunodeficiency virus (HIV), human papillomavirus (HPV), meningococcal disease, pneumococcal disease, measles, mumps, norovirus, polio, respiratory syncytial virus (RSV), rotavirus, rubella virus, shingles, West Nile virus, rabies virus, enterovirus, cytomegalovirus, herpes virus, varicella, Yellow fever, Zika virus, or any combination thereof.

[Embodiment AN] A method of any one of Embodiments [AJ] or [AK] above, or according to other embodiments of the invention, wherein the infectious disease is a bacterial infection.

[Embodiment AO] A method of any one of Embodiments [AJ] or [AK] above, or according to other embodiments of the invention, wherein the infectious disease is selected from streptococcal disease, staphylococcal disease, diphtheria, meningococcal disease, tetanus, pertussis, pneumococcal disease, bacterial food poisoning, sexually transmitted infections, tuberculosis, Lyme disease, botulism, or any combination thereof.

[Embodiment AP] A method of any one of Embodiments [AJ] or [AK] above, or according to other embodiments of the invention, wherein the infectious disease is a fungal infection.

[Embodiment AQ] A method of any one of Embodiments [AJ] or [AK] above, or according to other embodiments of the invention, wherein the infectious disease is selected from candidiasis, histoplasmosis, dermatophytosis, tinea pedis, aspergillosis, cryptococcal meningitis, coccidioidomycosis, or any combination thereof.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. A compound of the Formula (I) or (II) 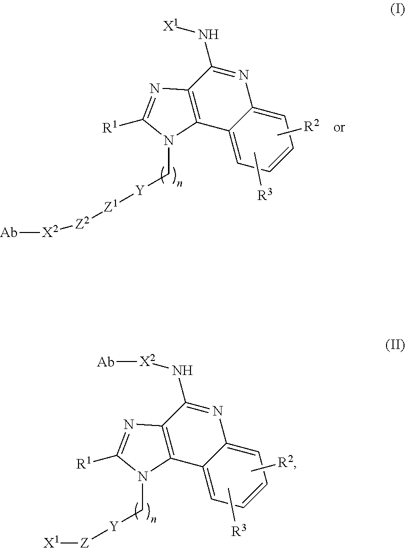
wherein:
R¹ is selected from C₁-C₁₀ alkyl, C₁-C₁₀ oxaalkyl, and C₁-C₁₀ azaalkyl;
R² and R³ are each independently selected from hydrogen, C₁-C₅ alkyl, and C₁-C₅ alkoxy;
n is 1 or 2
Y is independently selected from optionally substituted aryl and optionally substituted heteroaryl;
Z¹ is selected from —NR^{Z}—, —O—, —NR^{Z}C(O)—, —NR^{Z}C(O)—O—, and —NR^{Z}SO₂—;
Z² is absent, or is selected from (C₁-C₈)hydrocarbon-NH— and a 5- to 8-membered nitrogen-containing heterocycle, wherein a nitrogen of the heterocycle is attached to X²;
Z is independently selected from —NR^{Z}—, —NR^{Z}C(O)—, and —O—;
R^{Z} is independently selected in each instance from hydrogen, C₁-C₈ hydrocarbon, C₁-C₈ oxaalkyl, C₁-C₈ azaalkyl, heteroaryl, and a 5- to 8-membered heterocyclic ring;
X¹ is independently selected from R^{Z}, —C(O)—R^{Z}, —C(O)—O—R^{Z}, —C(O)—N—(R^{Z})₂, —(CH₂)ₖNR^{Z}C(O)—(C₁-C₆)alkyl, —(CH₂)ₖNR^{Z}C(O)—O—(C₁-C₄)alkyl, and —SO₂—R^{Z};
k is an integer from 1 to 8;
X² comprises cleavable or noncleavable linker; and
Ab comprises an antibody or an antibody fragment.

2. The compound according to claim 1, of the Formula (I) or (II) 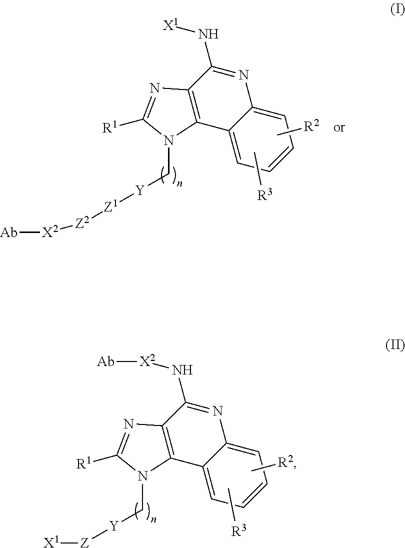
wherein:
R¹ is selected from C₁-C₁₀ alkyl, C₁-C₁₀ oxaalkyl, and C₁-C₁₀ azaalkyl;
R² and R³ are each independently selected from hydrogen, C₁-C₅ alkyl, and C₁-C₅ alkoxy;
n is 1 or 2
Y is independently selected from optionally substituted aryl and optionally substituted heteroaryl;
Z¹ is selected from —NR^{Z}—, —O—, —NR^{Z}C(O)—, —NR^{Z}C(O)—O—, and —NR^{Z}SO₂—;
Z² is absent, or is selected from (C₁-C₈)hydrocarbon-NH— and a 5- to 8-membered nitrogen-containing heterocycle, wherein a nitrogen of the heterocycle is attached to X²;
Z is selected from —NR^{Z}— and —O—;
R^{Z} is independently selected in each instance from hydrogen, C₁-C₈ hydrocarbon, C₁-C₈ oxaalkyl, C₁-C₈ azaalkyl, heteroaryl, and a 5- to 8-membered heterocyclic ring;
X¹ is independently selected from R^{Z}, —C(O)—R^{Z}, —C(O)—O—R^{Z}, —C(O)—N—(R^{Z})₂, and —SO₂—R^{Z};
X² comprises cleavable or noncleavable linker; and
Ab comprises an antibody or an antibody fragment.

3. (canceled)

4. A compound according to claim 1, wherein:
X² is L1-L2-(L3)ₚ-(L4)_{q}-(L5)ᵣ;
L1 is a conjugation moiety;
L2 is a spacer unit selected from branched or unbranched C₁-C₁₂ alkyl, a PEG selected from PEG1 to PEG12, 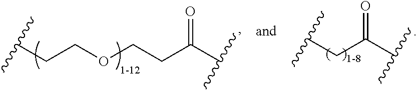
L3 is a peptide of 1 to 6 amino acids;
L4 is a self-immolative spacer;
L5 is carbonyl; and
p, q, and r are each independently selected from 0 and 1, wherein when p and q are each 0, r must be 0.

5. The compound according to claim 1, wherein said compound is of Formula (I), and:
R¹ is selected from n-butyl, —CH₂OH, and —CH₂OCH₂CH₃;
R² and R³ are each hydrogen;
n is 1;
Y is phenyl or pyridyl, each of which is unsubstituted or substituted with one or more of halogen, C₁-C₄ alkyl, C₁-C₄ alkoxy, C₁-C₄ haloalkyl, or C₁-C₄ haloalkoxy;
X¹ is hydrogen; and
Z¹ is —N(R^{Z})— or —O—.

6. The compound according to claim 1, wherein said compound is of Formula (II), and:
R¹ is selected from n-butyl, —CH₂OH, and —CH₂OCH₂CH₃;
R² and R³ are each hydrogen;
n is 1;
Y is phenyl or pyridyl, each of which is unsubstituted or substituted with one or more of halogen, C₁-C₄ alkyl, C₁-C₄ alkoxy, C₁-C₄ haloalkyl, or C₁-C₄ haloalkoxy;
X¹ is hydrogen; and
Z is —N(R^{Z})— or —O—.

7. The compound of claim 1, wherein the compound of Formula (I) is the compound of formulae (Ia), (Ib), (Ic), (Id), (Ie), (If), (Ig), or (Ih): 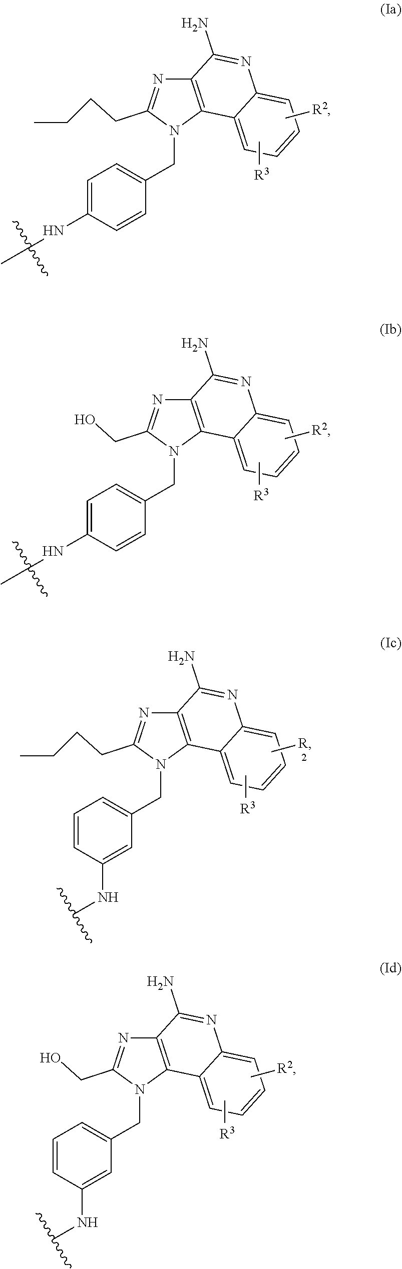 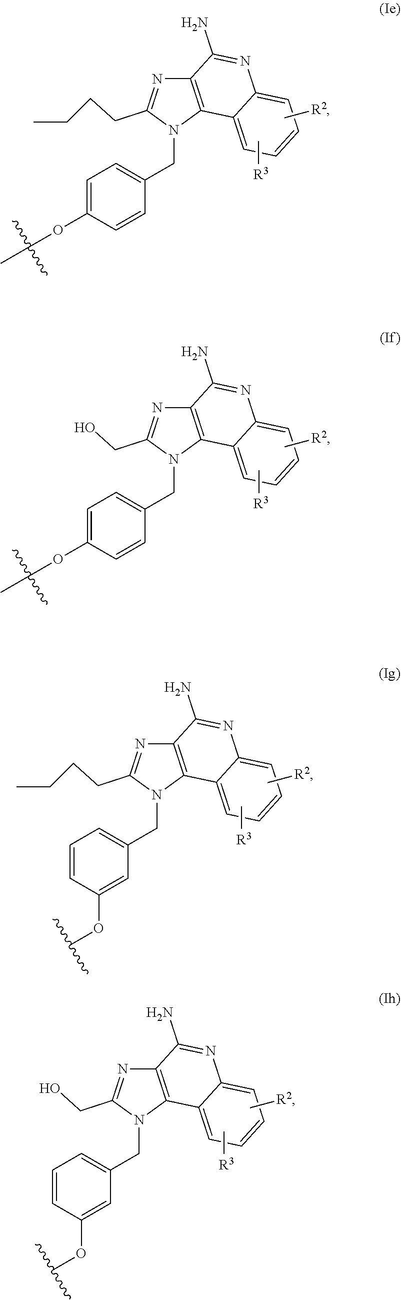
wherein [symbol] represents a point of attachment to X₂.

8. (canceled)

9. The compound of claim 1, wherein the compound of Formula (II) is the compound of formulae (IIa), (IIb), (IIc), (IId), (IIe), (IIf), (IIg), or (IIh): 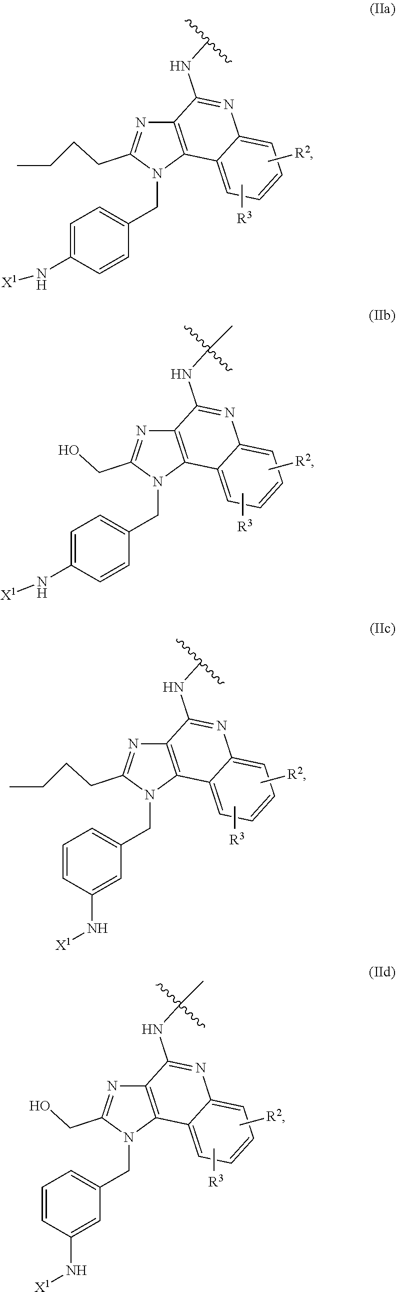 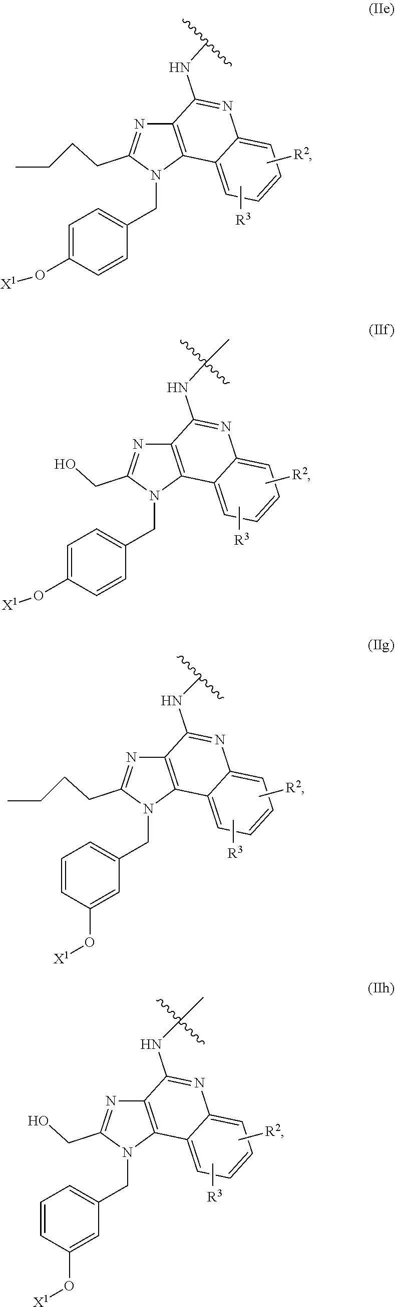
wherein [symbol] represents a point of attachment to X₂.

10. The compound of claim 4, wherein:
L1 is selected from: 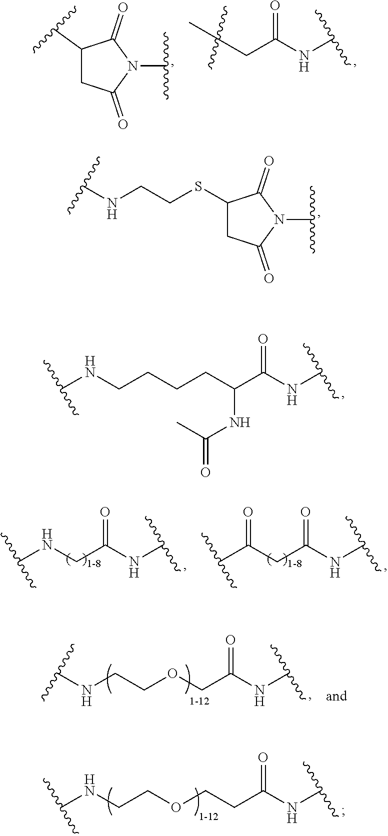
L2 is selected from 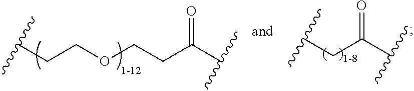
L3 is 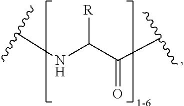
wherein R is an amino acid side chain;
L4 is selected from: 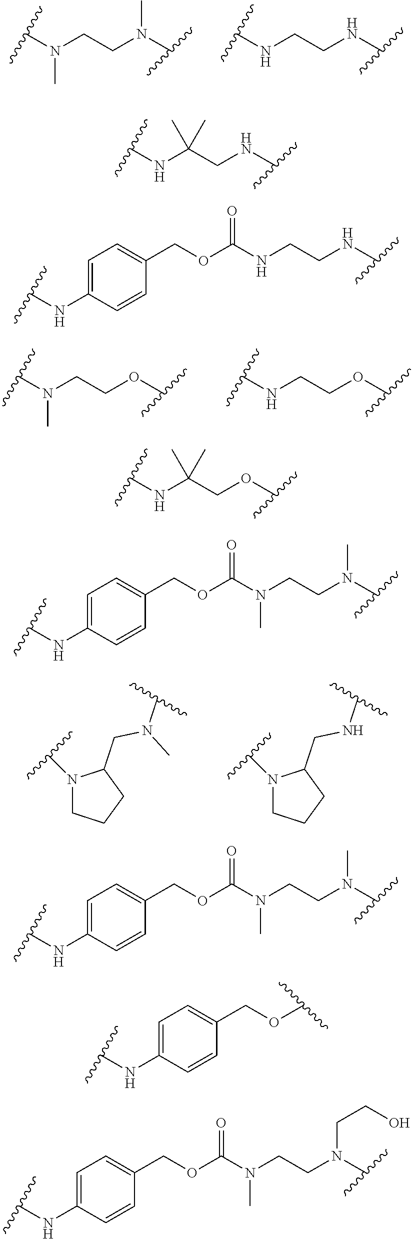 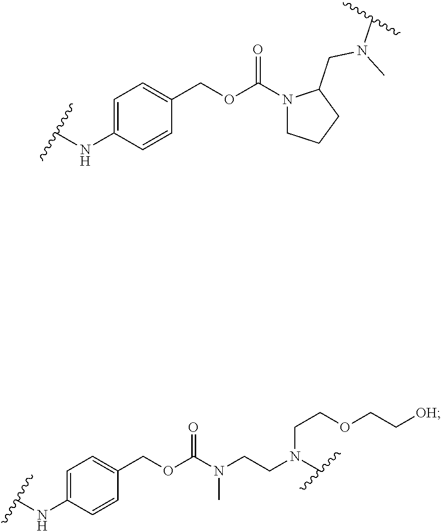 [SMILES: *Nc1ccc(COC(=O)N2CCCC2CN(*)C)cc1 ; *Nc1ccc(COC(=O)N(C)CCN(*)CCOCCO)cc1]
L5 is 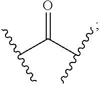 [SMILES: *C(*)=O]
and
p, q, and r are each independently 0 or 1, wherein when p and q are each 0, r must be 0.

11. The compound of claim 10, wherein L3 is selected from ValCit, GlyValCit, ValArg, PheLys, AlaAla, GlyGlyPheGly, AlaAlaAla, AlaAsn, AsnAsn, AsnAla, ValCitGlyPro, AsnGlyPro, AsnAsnGlyPro, Asn, GlyAsn, AsnAla, ProCitAla, ProAsnLeu, ProAsnAla, ProPheAla, ProPheGly, ProCitLeu, ProAsnPro, ProAsnSer, and ProAsnGly.

12. The compound of claim 10, wherein:
L1 is selected from: 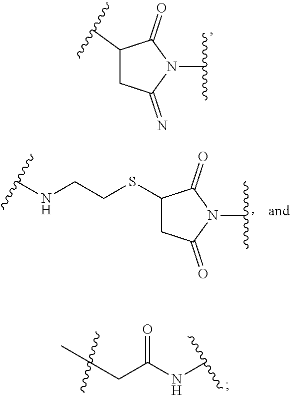 [SMILES: *C1CC(=N)N(*)C1=O ; *NCCSC1CC(=O)N(*)C1=O ; *CC(=O)N*]
L2 is 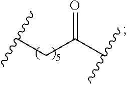
L3 is ValCit, GlyValCit, AsnAsn, Asn or AlaAla;
L4 is selected from: 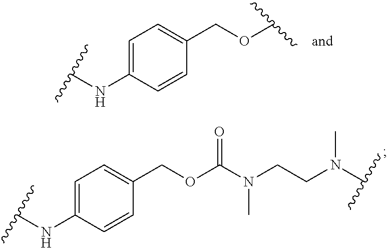 [SMILES: *Nc1ccc(CO*)cc1 ; *Nc1ccc(COC(=O)N(C)CCN(*)C)cc1]
L5 is 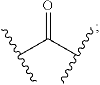 [SMILES: *C(*)=O]
and
p, q, and r are each 0 or p, q, and r are each 1.

13. The compound according to claim 1, wherein X² is attached to Ab through a cysteine residue of Ab, a lysine residue of Ab, or a glutamine residue of Ab; or wherein Ab is a tumor targeting antibody, an antibody fragment, a bispecific antibody or antibody fragment, a monoclonal antibody, a chimeric antibody, or a humanized antibody.

14-15. (canceled)

16. A compound of the Formula (III) 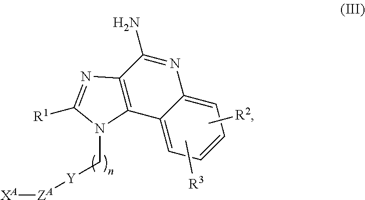
wherein:
R¹ is selected from C₁-C₁₀ alkyl, C₁-C₁₀ oxaalkyl, and C₁-C₁₀ azaalkyl;
R² and R³ are each independently selected from hydrogen, C₁-C₅ alkyl, and C₁-C₅ alkoxy;
n is 1 or 2;
Y is selected from optionally substituted aryl and optionally substituted heteroaryl;
Z^{A} is selected from —NR^{Z}—, —NR^{Z}C(O)—, —NR^{Z}C(O)—O—, —NR^{Z}C(O)—(CH₂)ₖ—NH—, —NR^{Z}C(O)—(CH₂)ₖ—O—, —C(O)—O—(CH₂)ₖ—O—, —NR^{Z}C(O)—(CH₂)ₖ—N(CH₃)—, —NR^{Z}C(O)—O—(CH₂)ₖ—NH—, —NR^{Z}C(O)—(CH₂)ₖ—NH—C(O)—O—, and —NR^{Z}SO₂—;
k is an integer from 1 to 8;
R^{Z} is selected from hydrogen, C₁-C₈ hydrocarbon, C₁-C₈ oxaalkyl, C₁-C₈ azaalkyl, heteroaryl, and a 5- to 8-membered heterocyclic ring; and
X^{A} is selected from hydrogen, C₁-C₁₀ alkyl, and —C(O)CH₃,
wherein the following compound is excluded:

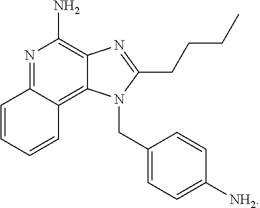 [SMILES: CCCCc1nc2c(N)nc3ccccc3c2n1Cc1ccc(N)cc1]

17. The compound of claim 16, wherein:
R¹ is selected from n-butyl, —CH₂OH, and —CH₂OCH₂CH₃;
R² and R³ are each hydrogen;
n is 1;
Y is phenyl or pyridyl, each of which is unsubstituted or substituted with one or more of halogen, C₁-C₄ alkyl, C₁-C₄ alkoxy, C₁-C₄ haloalkyl, or C₁-C₄ haloalkoxy; and
R^{Z}, when present, is hydrogen.

18. The compound of claim 17, wherein R¹ is n-butyl and Y is unsubstituted phenyl; and/or wherein Z^{A} is selected from —NR^{Z}—, —NR^{Z}C(O)—, —NRC(O)—O—, —NR^{Z}C(O)—(CH₂)ₖ—NH—, —N C(O)—O—(CH₂)ₖ—NH—, —N C(O)—(CH₂)ₖ—NH—C(O)—O—, and —NR^{Z}SO₂—.

19. (canceled)

20. The compound of claim 19, wherein Z^{A}—X^{A} is selected from —NHC(O)O(C₁-C₄)alkyl, —NH₂, —NHC(O)(CH₂)ₖNH₂, —NHC(O)(CH₂)ₖNH—C(O)O(C₁-C₄)alkyl, and —NHC(O)(C₁-C₄)alkyl.

21. A pharmaceutical composition comprising the compound of any claim 1 and a pharmaceutically acceptable carrier, diluent, or excipient.

22. (canceled)

23. A method for stimulating an immune response in a subject, the method comprising administering a therapeutically effective amount of the compound of claim 1 under conditions effective to stimulate an immune response.

24-25. (canceled)

26. A method for inducing an anti-tumor immune response in a subject, the method comprising administering to a therapeutically effective amount of the compound of claim 1 under conditions effective to induce an anti-tumor immune response.

27-28. (canceled)

29. A method for treating a tumor or abnormal cell proliferation, said method comprising administering a therapeutically effective amount of the compound of claim 1, under conditions effective to treat a tumor or abnormal cell proliferation.

30-31. (canceled)

32. A method for treating an infectious disease, said method comprising: administering a therapeutically effective amount of the compound of claim 1, under conditions effective to treat an infectious disease.

33-36. (canceled)
